(19) United States
(12) Patent Application Publication
(10) Pub. No.: US 2007/0031701 A1
(43) Pub. Date: Feb. 8, 2007

(54) Carbazole derivative, light-emitting element material obtained by using carbazole derivative, light-emitting element, and electronic device

(75) Inventors: Harue Nakashima, Atsugi (JP); Sachiko Kawakami, Isehara (JP); Kumi Kojima, Machida (JP); Ryoji Nomura, Yamato (JP); Nobuharu Ohsawa, Zama (JP)

Correspondence Address: ERIC ROBINSON, PMB 955, 21010 SOUTHBANK ST., POTOMAC FALLS, VA, 20165, US

(73) Assignee: Semiconductor Energy Laboratory Co., Ltd., Atsugi-shi (JP)

(21) Appl. No.: 11/494,538

(22) Filed: Jul. 28, 2006

(30) Foreign Application Priority Data
Aug. 4, 2005 (JP) 2005-226225

(51) Int. Cl. H01L 51/54; H05B 33/14; C07D 209/88

(52) U.S. Cl. 428/690; 428/917; 313/504; 257/E51.049; 257/E51.051; 564/434; 548/440; 548/442

(57) ABSTRACT

It is an object to provide a carbazole derivative which is useful as a raw material for forming a light-emitting element material which has resistance to repeated oxidation reactions. It is an object to provide a carbazole derivative represented by a general formula (G-1). In the general formula (G-1), each of Ar¹ and Ar² represents an aryl group having 1 to 12 carbon atoms such as phenyl, biphenyl, or naphthyl. Also, R¹ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. Further, the aryl group may have a substituent, or is not required to have a substituent.

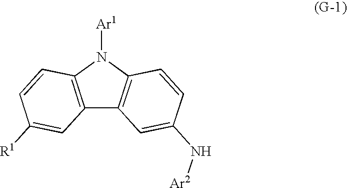

### TECHNICAL FIELD

The present invention relates to a carbazole derivative which can be used as a raw material to obtain a light-emitting element material. The present invention also relates to a light-emitting element material obtained by using the carbazole derivative, and a light-emitting element and an electronic device manufactured by using the light-emitting element material.

### BACKGROUND ART

In recent years, practical application of a light-emitting device using a light-emitting element as a pixel has been advanced. Such a light-emitting device is incorporated into a display portion of an electronic device such as a portable music reproducing device besides a cellular phone and a camera, and the light-emitting device serves as a medium which provides an operation screen or a screen for reproducing an image such as a photograph. A device which utilizes electromotive force from a battery as in these electronic devices is attempted to be continuously used for a long time; therefore, it is essential that power consumption of a light-emitting device is suppressed. Also, it is essential that a light-emitting element with good light emission efficiency is developed in order to obtain a light-emitting device with low power consumption.

In general, a light-emitting element has a structure in which a light-emitting layer is provided between electrodes. Layers having various functions such as a hole transporting layer, an electron transporting layer, a hole injecting layer, and an electron injecting layer are provided between the electrodes in addition to the light-emitting layer, and a material suitable for forming these layers has been developed. For example, a carbazole derivative which is suitable for forming a hole transporting layer is disclosed in Patent Document 1.

In a light-emitting element, current flows between electrodes by hopping conduction. Therefore, a material used for forming the light-emitting element is required to have resistance to repeated oxidation reactions and/or repeated reduction reactions. In addition, as for a material used for forming a hole injecting layer (hereinafter, referred to as a hole injecting material), holes are required to be easily injected to the hole injecting layer from an anode and the injected holes are required to be sufficiently transported. Therefore, a hole injecting material is required to have ionization potential in which a difference from a work function of the anode is small and a favorable hole transporting property.
Patent Document 1: Japanese Patent Application Laid-Open No. 2001-220380

### DISCLOSURE OF INVENTION

It is an object of the present invention to provide a carbazole derivative which is useful as a raw material for manufacturing a light-emitting element material having resistance to repeated oxidation reactions.

It is another object of the present invention to provide a light-emitting element material having resistance to repeated oxidation reactions.

It is another object of the present invention to provide a light-emitting element exhibiting blue light emission with favorable chromaticity, a light-emitting device, and an electronic device.

One feature of the present invention is a carbazole derivative represented by the following general formula (G-1).

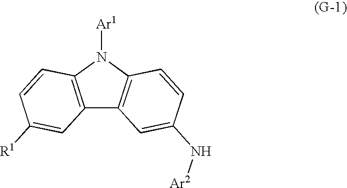

In the general formula (G-1), each of Ar¹ and Ar² represents an aryl group having 1 to 12 carbon atoms such as phenyl, biphenyl, or naphthyl. R¹ represents an alkyl group having 1 to 4 carbon atoms such as hydrogen, methyl, ethyl, or tert-butyl, or an aryl group having 6 to 12 carbon atoms such as phenyl, biphenyl, or naphthyl. It is to be noted that the aryl group may have a substituent, or is not required to have a substituent.

Another feature of the present invention is a carbazole derivative represented by the following general formula (G-2).

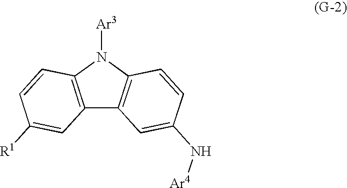

In the general formula (G-2), each of Ar³ and Ar⁴ represents an aryl group having 1 to 12 having carbon atoms such as phenyl, biphenyl, or naphthyl. It is to be noted that the aryl group may have a substituent, or is not required to have a substituent.

Another feature of the present invention is a light-emitting element material represented by the following general formula (G-3).

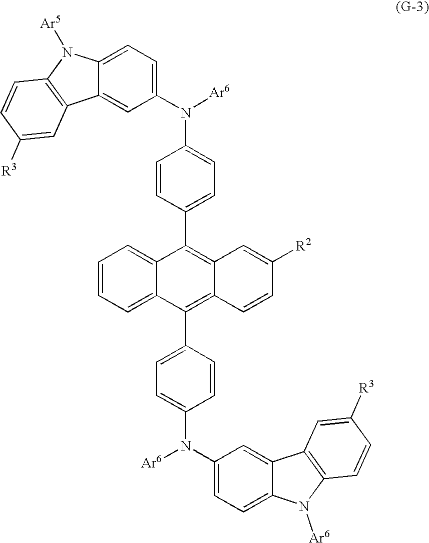

In the general formula (G-3), each of Ar⁵ and Ar⁶ represents an aryl group having 1 to 12 carbon atoms such as phenyl, biphenyl, or naphthyl. R² represents any one of hydrogen, methyl, and tert-butyl. R³ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that the aryl group may have a substituent, or is not required to have a substituent.

Another feature of the present invention is a light-emitting element material represented by the general formula (G-4).

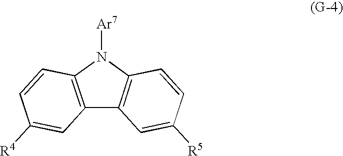

In the general formula (G-4), Ar⁷ represents an aryl group having 1 to 12 carbon atoms such as phenyl, biphenyl, or naphthyl. Each of R⁴ and R⁵ represents hydrogen or a group represented by a general formula (G-5), and one of them is represented by the following general formula (G-5). In the general formula (G-5), each of Ar⁸ and Ar⁹ represents an aryl group having 1 to 12 carbon atoms such as phenyl, biphenyl, or naphthyl. R⁶ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that the aryl group may have a substituent, or is not required to have a substituent.

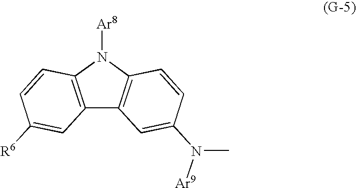

Another feature of the present invention is a light-emitting element having a layer formed by using the light-emitting element material represented by the above-described general formula (G-3) or (G-4) between electrodes.

Another feature of the present invention is a light-emitting element having a light-emitting layer between electrodes, where the light-emitting layer includes a light-emitting substance represented by the following general formula (G-6) and a host having higher ionization potential than the light-emitting substance and a larger energy gap than the light-emitting substance. It is preferable that the host have a higher transporting property of electrons than holes.

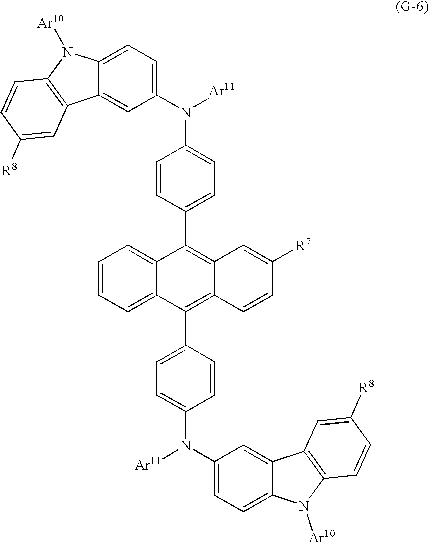

In the general formula (G-6), each of Ar¹⁰ and Ar¹¹ represents an aryl group having 1 to 12 carbon atoms such as phenyl, biphenyl, or naphthyl. R⁷ represents any one of hydrogen, methyl, and tert-butyl. R⁸ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that the aryl group may have a substituent, or is not required to have a substituent.

Another feature of the present invention is a light-emitting element having a layer containing a carbazole derivative represented by the following general formula (G-7) between a first electrode and a second electrode to be in contact with the first electrode, where light is emitted when voltage is applied so that potential of the first electrode is higher than that of the second electrode.

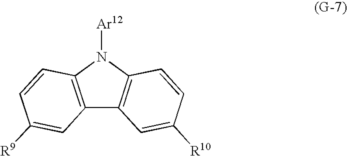

In the general formula (G-7), Ar¹² represents an aryl group having 1 to 12 carbon atoms such as phenyl, biphenyl, or naphthyl. Each of R⁹ and R¹⁰ represents hydrogen or a group represented by a general formula (G-8), and one of them is represented by the following general formula (G-8). In the general formula (G-8), each of Ar¹³ and Ar¹⁴ represents an aryl group having 1 to 12 carbon atoms such as phenyl, biphenyl, or naphthyl. R¹¹ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. The aryl group may have a substituent, or is not required to have a substituent.

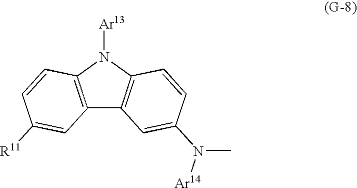

Another feature of the present invention is a light-emitting device including a light-emitting element manufactured by using a light-emitting element material represented by either the above-described general formula (G-3) or (G-4).

Another feature of the present invention is an electronic device having a light-emitting device including a light-emitting element manufactured by using the light-emitting element material represented by either the above-described general formula (G-3) or (G-4) for a display portion or a lighting portion.

By implementing the present invention, a carbazole derivative which is useful for manufacturing a light-emitting element material having excellent resistance to repeated oxidation reactions can be obtained. Also, by implementing the present invention, a light-emitting element material having excellent resistance to repeated oxidation reactions can be obtained. In addition, by implementing the present invention, a light-emitting device which has resistance to repeated oxidation reactions and can emit light in a favorable state for a long time can be obtained. Moreover, by implementing the present invention, an electronic device which can perform a display action or lighting favorably for a long time can be obtained.

By implementing the present invention, a carbazole derivative which can exhibit blue light emission with favorable chromaticity and is useful for forming a light-emitting element material which is useful to be used as a light-emitting substance can be obtained. By implementing the present invention, a light-emitting element material which can exhibit blue light emission with favorable chromaticity can be obtained. In addition, by implementing the present invention, a light-emitting device which exhibits blue light emission with favorable chromaticity and displays an image with excellent colors can be obtained. Moreover, by implementing the present invention, an electronic device which exhibits blue light emission with favorable chromaticity and displays an image with excellent colors can be obtained.

### BRIEF DESCRIPTION OF DRAWINGS

In the accompanying drawings:

FIG. 1 is a view explaining a light-emitting element of the present invention;

FIG. 2 is a view explaining a light-emitting element of the present invention;

FIG. 3 is a view explaining a light-emitting device to which the present invention is applied;

FIG. 4 is a view explaining a circuit included in a light-emitting device to which the present invention is applied;

FIG. 5 is a view explaining a frame operation, in accordance with passage of time, of a light-emitting device to which the present invention is applied;

FIGS. 6A to 6C are cross-sectional views of a light-emitting device to which the present invention is applied;

FIGS. 7A and 7B are views each explaining a circuit included in a light-emitting device to which the present invention is applied;

FIGS. 8A to 8D are views of electronic devices to which the present invention is applied;

FIG. 9 is a view of an electronic device to which the present invention is applied;

FIGS. 10A and 10B are ¹H-NMR charts of a carbazole derivative synthesized in Synthesis Example 1 (abbreviation: PCA);

FIGS. 11A and 11B are ¹H-NMR charts of a carbazole derivative synthesized in Synthesis Example 2 (abbreviation: PCN);

FIGS. 12A and 12B are ¹H-NMR charts of an anthracene derivative synthesized in Synthesis Example 3 (abbreviation: PCABPA);

FIG. 13 is a graph showing absorption spectra of PCABPA;

FIG. 14 is a graph showing light emission spectra of PCABPA;

FIGS. 15A and 15B are graphs showing measurement results of PCABPA by a cyclic voltammetry (CV);

FIGS. 16A and 16B are ¹H-NMR charts of a carbazole derivative synthesized in Synthesis Example 4 (abbreviation: PCzPCA1);

FIG. 17 is a graph showing a result of thermogravimetry-differential thermal analysis of PCzPCA1;

FIG. 18 is a graph showing absorption spectra of PCzPCA1;

FIG. 19 is a graph showing light emission spectra of PCzPCA1;

FIG. 20 is a graph showing measurement results of PCzPCA1 by a cyclic voltammetry (CV);

FIG. 21 is a graph showing a measurement result of PCzPCA1 by using a differential scanning calorimetry;

FIGS. 22A and 22B are ¹H-NMR charts of a carbazole derivative synthesized in Synthesis Example 5 (abbreviation: PCzPCA2);

FIG. 23 is a graph showing a result of thermogravimetry-differential thermal analysis of PCzPCA2;

FIG. 24 is a graph showing absorption spectra of PCzPCA2;

FIG. 25 is a graph showing light emission spectra of PCzPCA2;

FIG. 26 is a graph showing measurement results of PCzPCA2 by a cyclic voltammetry (CV);

FIG. 27 is a graph showing a measurement result of PCzPCA2 by using a differential scanning calorimetry;

FIGS. 28A and 28B are ¹H-NMR charts of a carbazole derivative synthesized in Embodiment 3 (abbreviation: PCzPCN1);

FIG. 29 is a graph showing a result of thermogravimetry-differential thermal analysis of PCzPCN1;

FIG. 30 is a graph showing absorption spectra of PCzPCN1;

FIG. 31 is a graph showing light emission spectra of PCzPCN1;

FIG. 32 is a graph showing measurement results of PCzPCN1 by a cyclic voltammetry (CV);

FIG. 33 is a graph showing a measurement result of PCzPCN1 by using a differential scanning calorimetry;

FIG. 34 is a view explaining a light-emitting element manufactured in embodiments;

FIG. 35 is a graph showing a voltage-luminance characteristic of a light-emitting element manufactured in Embodiment 4;

FIG. 36 is a view showing a luminance-current efficiency characteristic of a light-emitting element manufactured in Embodiment 4;

FIG. 37 is a view showing a light emission spectrum of a light-emitting element manufactured in Embodiment 4;

FIG. 38 is a view showing a voltage-luminance characteristic of a light-emitting element manufactured in Embodiment 5;

FIG. 39 is a view showing a luminance-current efficiency characteristic of a light-emitting element manufactured in Embodiment 5;

FIG. 40 is a view showing a light emission spectrum of a light-emitting element manufactured in Embodiment 5;

FIG. 41 is a view showing a voltage-luminance characteristic of a light-emitting element manufactured in Embodiment 6;

FIG. 42 is a view showing a luminance-current efficiency characteristic of a light-emitting element manufactured in Embodiment 6;

FIG. 43 is a view showing a light emission spectrum of a light-emitting element manufactured in Embodiment 6;

FIG. 44 is a view showing a voltage-luminance characteristic of a light-emitting element manufactured in Embodiment 7;

FIG. 45 is a view showing a luminance-current efficiency characteristic of a light-emitting element manufactured in Embodiment 7;

FIG. 46 is a view showing a light emission spectrum of a light-emitting element manufactured in Embodiment 7;

FIG. 47 is a view showing a voltage-luminance characteristic of a light-emitting element manufactured in Embodiment 8;

FIG. 48 is a view showing a luminance-current efficiency characteristic of a light-emitting element manufactured in Embodiment 8;

FIG. 49 is a view showing a light emission spectrum of a light-emitting element manufactured in Embodiment 8;

FIG. 50 is a view showing a voltage-luminance characteristic of a light-emitting element manufactured in Embodiment 9;

FIG. 51 is a view showing a luminance-current efficiency characteristic of a light-emitting element manufactured in Embodiment 9;

FIG. 52 is a view showing a light emission spectrum of a light-emitting element manufactured in Embodiment 9;

FIG. 53 is a view showing a voltage-luminance characteristic of a light-emitting element manufactured in Embodiment 10;

FIG. 54 is a view showing a luminance-current efficiency characteristic of a light-emitting element manufactured in Embodiment 10;

FIG. 55 is a view showing a light emission spectrum of a light-emitting element manufactured in Embodiment 10;

FIG. 56 is a view showing a voltage-luminance characteristic of a light-emitting element manufactured in Embodiment 11;

FIG. 57 is a view showing a luminance-current efficiency characteristic of a light-emitting element manufactured in Embodiment 11;

FIG. 58 is a view showing a light emission spectrum of a light-emitting element manufactured in Embodiment 11;

FIG. 59 is a view showing a voltage-luminance characteristic of a light-emitting element manufactured in Embodiment 12;

FIG. 60 is a view showing a luminance-current efficiency characteristic of a light-emitting element manufactured in Embodiment 12;

FIG. 61 is a view showing a light emission spectrum of a light-emitting element manufactured in Embodiment 12;

FIG. 62 is a view showing a voltage-luminance characteristic of a light-emitting element manufactured in Embodiment 13;

FIG. 63 is a view showing a luminance-current efficiency characteristic of a light-emitting element manufactured in Embodiment 13;

FIG. 64 is a view showing a light emission spectrum of a light-emitting element manufactured in Embodiment 13;

FIG. 65 is a view showing a voltage-luminance characteristic of a light-emitting element manufactured in Embodiment 14;

FIG. 66 is a view showing a luminance-current efficiency characteristic of a light-emitting element manufactured in Embodiment 14;

FIG. 67 is a view showing a light emission spectrum of a light-emitting element manufactured in Embodiment 14;

FIG. 68 is a view showing a voltage-luminance characteristic of a light-emitting element manufactured in Embodiment 15;

FIG. 69 is a view showing a luminance-current efficiency characteristic of a light-emitting element manufactured in Embodiment 15;

FIG. 70 is a view showing a light emission spectrum of a light-emitting element manufactured in Embodiment 15;

FIGS. 71A and 71B are views showing a relative value of luminance with accumulation of light emission time and voltage with accumulation of light emission time of a light-emitting element manufactured in Embodiment 6;

FIGS. 72A and 72B are views showing a relative value of luminance with accumulation of light emission time and voltage with accumulation of light emission time of a light-emitting element manufactured in Embodiment 7;

FIGS. 73A and 73B are ¹H-NMR charts of a carbazole derivative synthesized in Synthesis Example 1 (abbreviation: PCA);

FIGS. 74A and 74B are ¹³C-NMR charts of a carbazole derivative synthesized in Synthesis Example 1 (abbreviation: PCA);

FIGS. 75A and 75B are ¹H-NMR charts of a carbazole derivative synthesized in Embodiment 17 (abbreviation: BCzBCA1);

FIGS. 76A and 76B are ¹³C-NMR charts of a carbazole derivative synthesized in Embodiment 17 (abbreviation: BCzBCA1);

FIG. 77 is a view showing an absorption spectrum of BCzBCA1;

FIG. 78 is a view showing a light emission spectrum of BCzBCA1 ;

FIG. 79 is a view showing an absorption spectrum of BCzBCA1;

FIG. 80 is a view showing a light emission spectrum of BCzBCA1;

FIG. 81 is a view showing a measurement result of BCzBCA1 by using a differential scanning calorimetry;

FIG. 82 is a view showing a voltage-luminance characteristic of a light-emitting element manufactured in Embodiment 18;

FIG. 83 is a view showing a luminance-current efficiency characteristic of a light-emitting element manufactured in Embodiment 18;

FIG. 84 is a view showing a light emission spectrum of a light-emitting element manufactured in Embodiment 18;

FIG. 85 is a view showing a voltage-luminance characteristic of a light-emitting element manufactured in Embodiment 19;

FIG. 86 is a view showing a luminance-current efficiency characteristic of a light-emitting element manufactured in Embodiment 19;

FIG. 87 is a view showing a light emission spectrum of a light-emitting element manufactured in Embodiment 19;

FIG. 88 is a view showing a voltage-luminance characteristic of a light-emitting element manufactured in Embodiment 20;

FIG. 89 is a view showing a luminance-current efficiency characteristic of a light-emitting element manufactured in Embodiment 20; and

FIG. 90 is a view showing a light emission spectrum of a light-emitting element manufactured in Embodiment 20.

### BEST MODE FOR CARRYING OUT THE INVENTION

Hereinafter, one mode of the present invention will be described. However, the present invention can be carried out in many different modes, and it is easily understood by those skilled in the art that modes and details can be modified in various ways without departing from the purpose and the scope of the present invention. Therefore, the present invention should not be interpreted by being limited to the description in embodiment modes and embodiments.

### Embodiment Mode 1

One mode of a carbazole derivative of the present invention and a manufacturieng method thereof will be explained.

As specific modes of the carbazole derivative of the present invention, a carbazole derivatives represented by the following structural formulas (1) to (44) can be given. However, the carbazole derivative of the present invention is not limited to those represented by the following formulas, and the carbazole derivative may have a structure which is different from those represented by the following formulas.

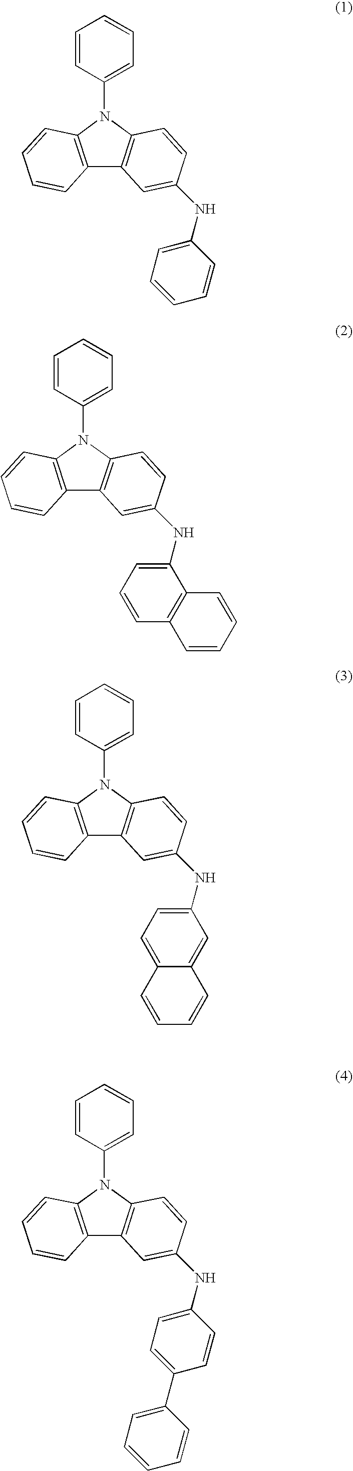

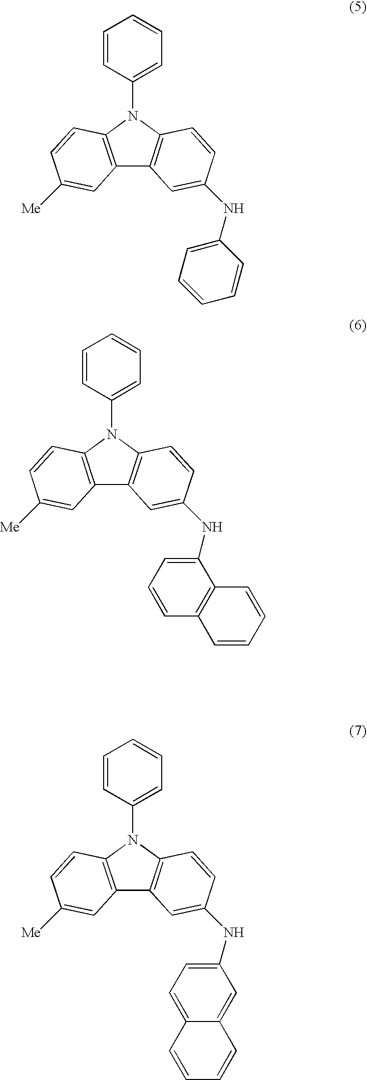

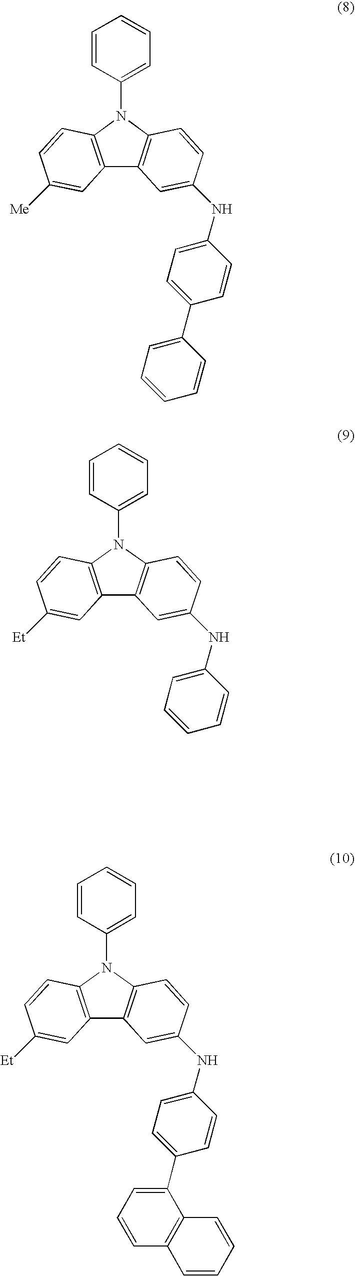

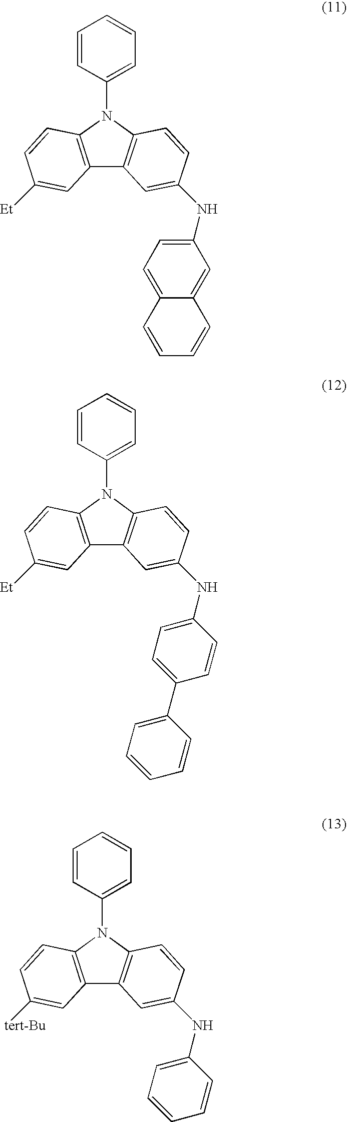

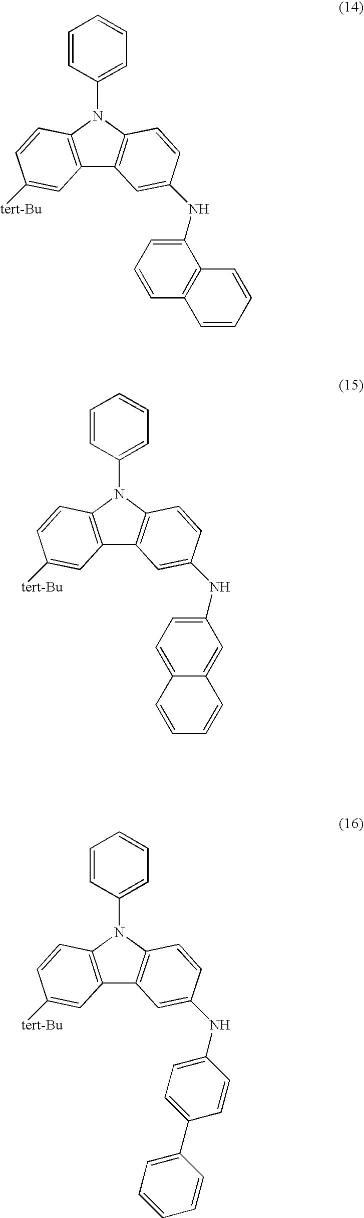

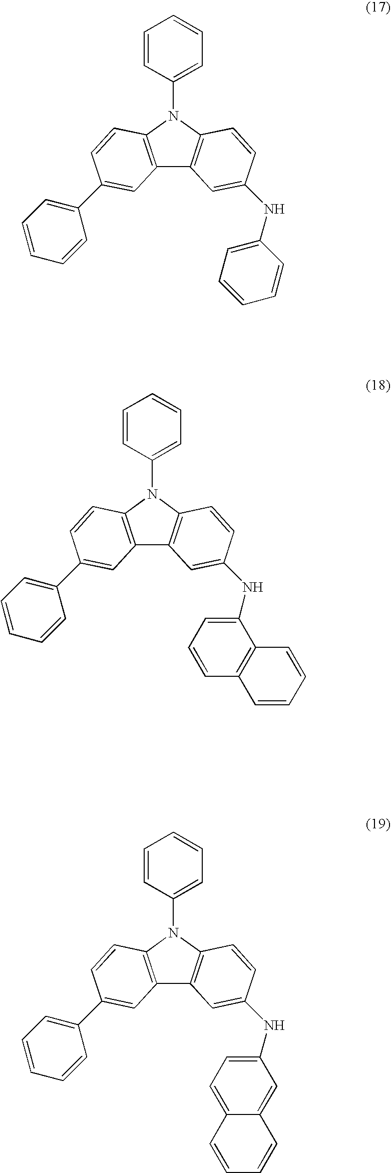

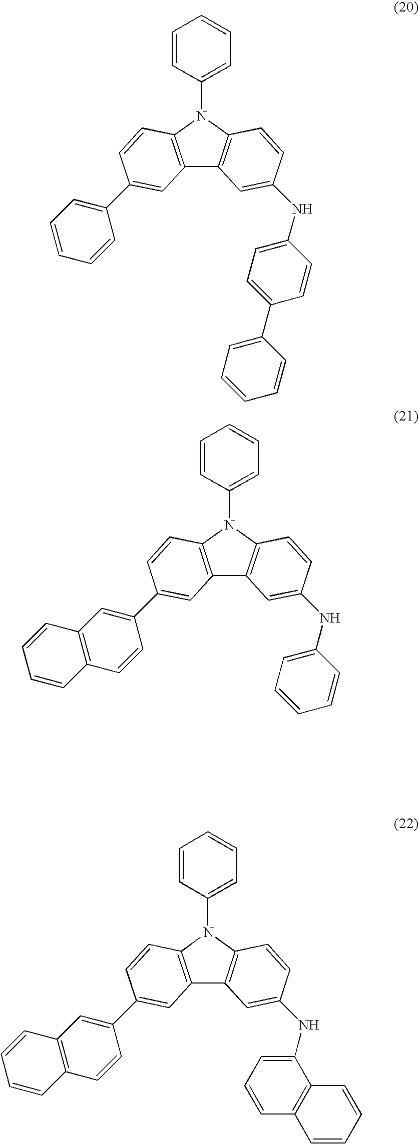

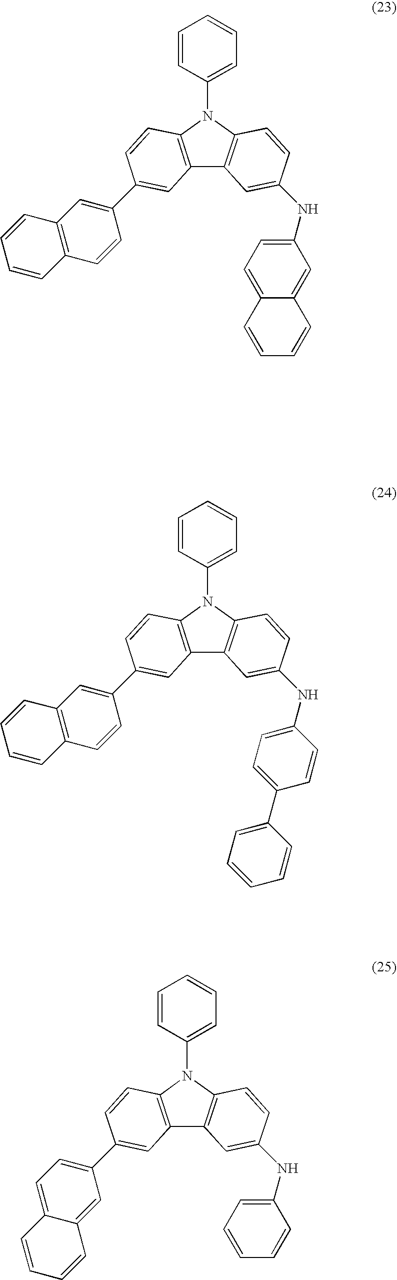

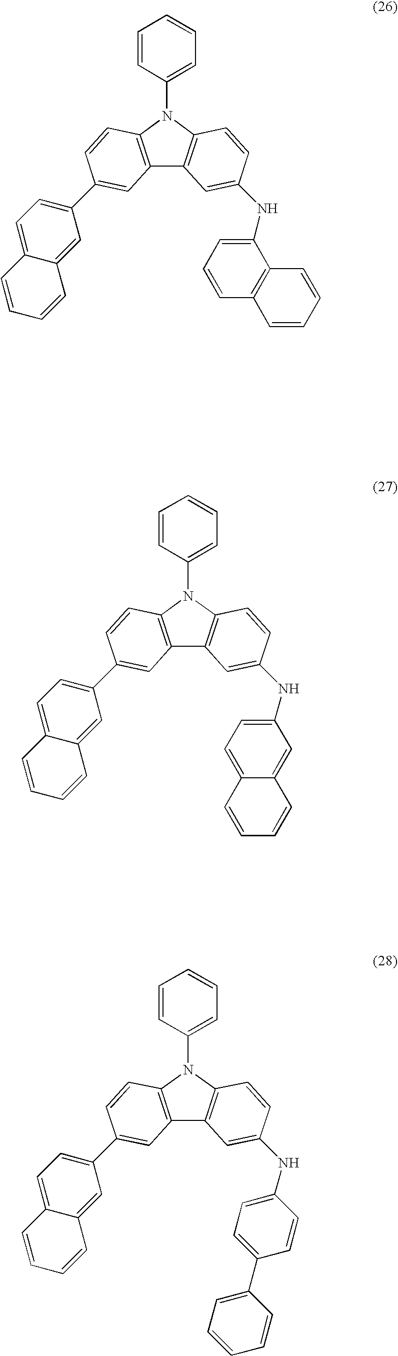

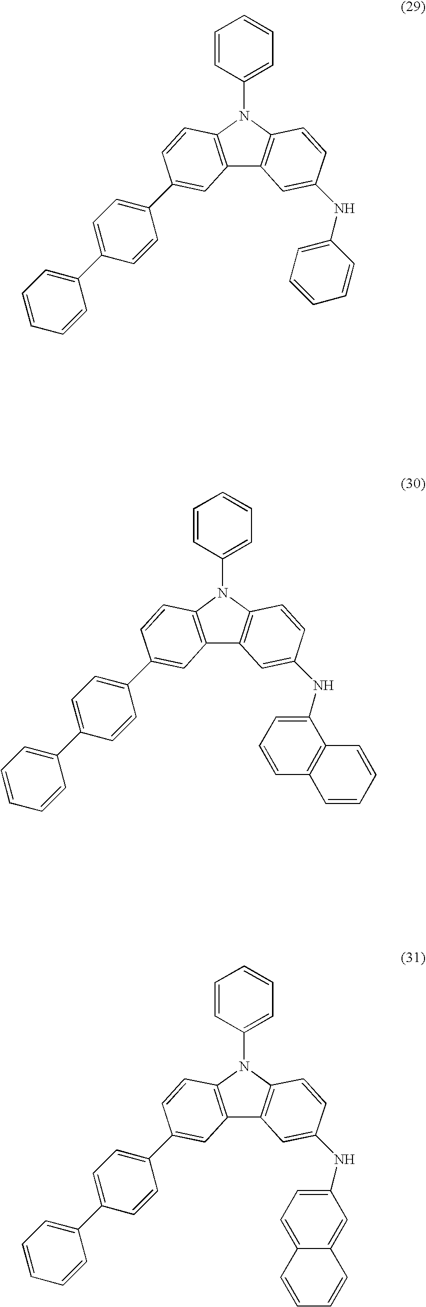

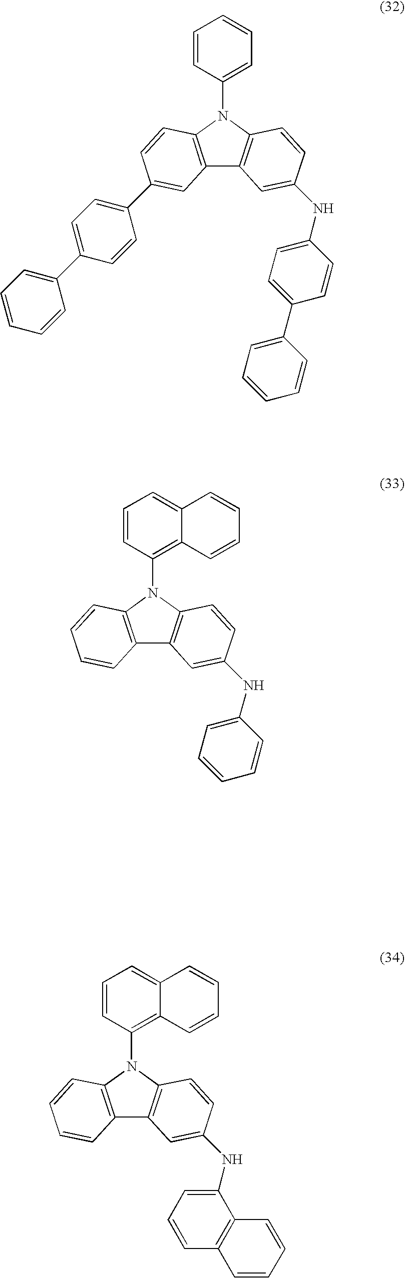

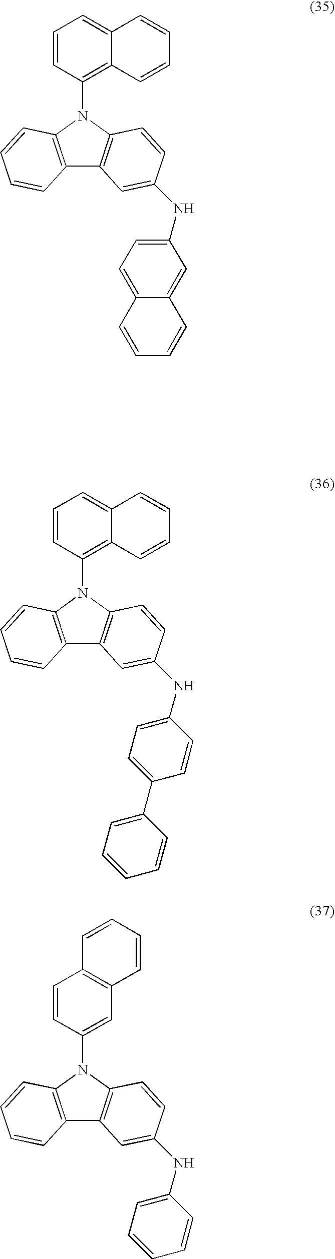

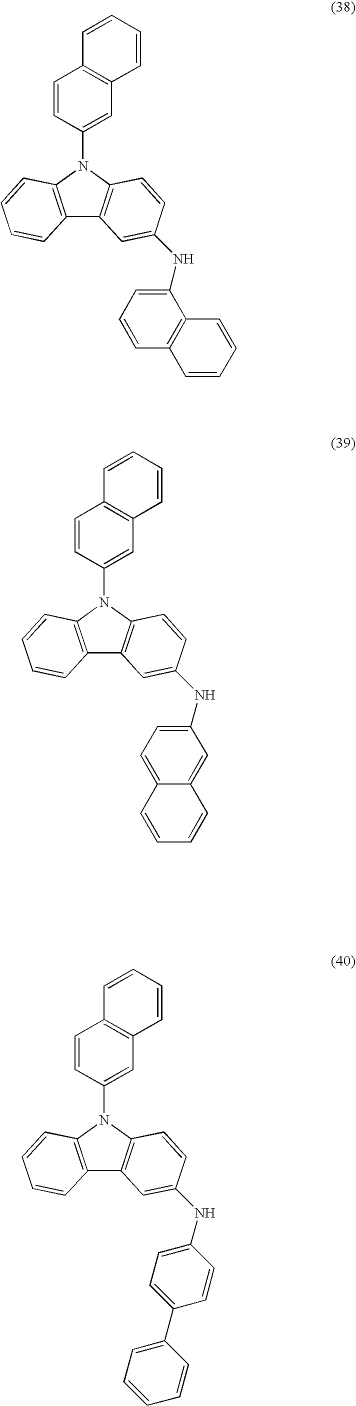

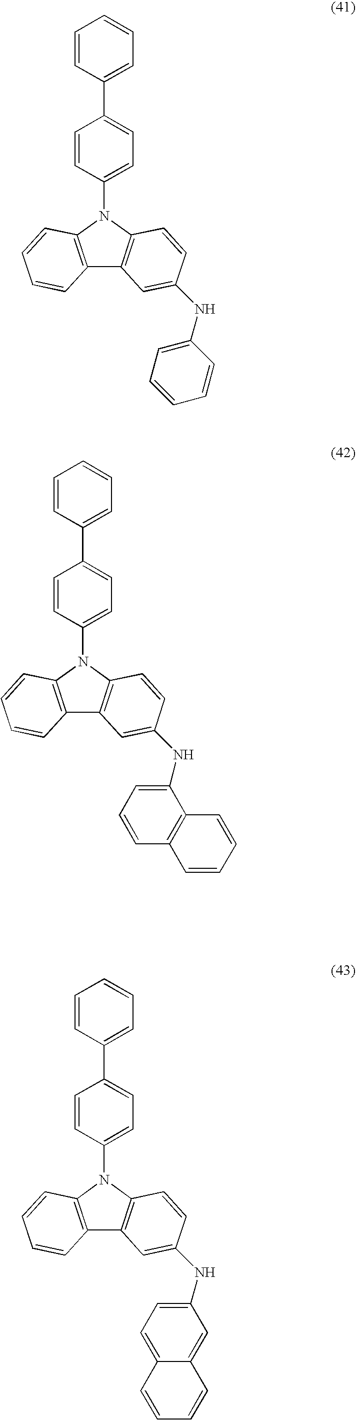

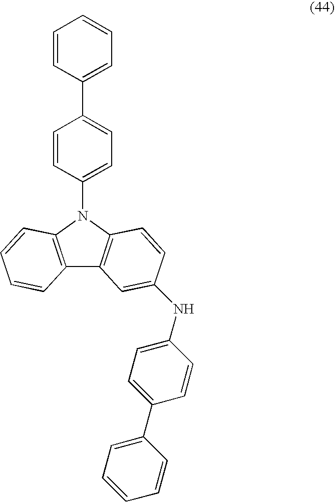

As shown by a synthesis scheme (a-1), hydrogen of three or six position of a compound containing carbazole in a skeleton is substituted for bromo or iodo by using N-bromosuccinimide (abbreviation: NBS), N-iodosuccinimide (NIS), or the like (it is to be noted that reaction time can be shortened by substituting for iodo) to synthesize a compound A. Thereafter, coupling reaction of the compound A and aryl amine is performed by using a metal catalyst such as a palladium catalyst. Accordingly, the carbazole derivative of the present invention, which is represented by the following general formula (G-9) and more specifically by structural formulas (1) to (44) is obtained. Further, although there are no particular limitations to a palladium catalyst, Pd(dba)₂ is preferable.

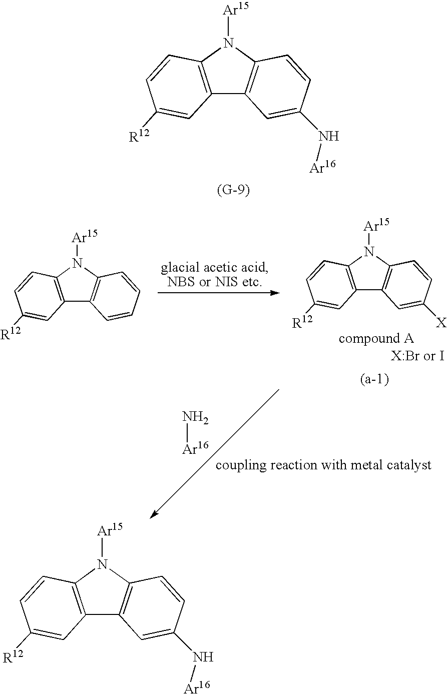

In the general formula (G-9) and the synthesis scheme (a-1), each of Ar¹⁵ and Ar¹⁶ represents an aryl group having 1 to 12 carbon atoms such as phenyl, biphenyl, or naphthyl. In addition, R¹² represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that these aryl groups may each have a substituent, or is not required to have a substituent.

Further, a synthesis method of the carbazole derivative of the present invention is not limited to the synthesis method represented by the synthesis scheme (a-1), and other synthesis methods may also be used.

The carbazole derivative of the present invention described above is highly useful as a raw material for forming a light-emitting element material having excellent resistance to repeated oxidation reactions.

### Embodiment Mode 2

One mode of a manufacturing method of a light-emitting element material using a carbazole derivative of the present invention will be explained.

As shown by the following synthesis scheme (b-1), coupling reaction of a carbazole derivative represented by a general formula (G-9) and a compound B having a diphenyl anthracene skeleton is performed by using a metal catalyst such as a palladium catalyst; accordingly, an anthracene derivative represented by the following general formula (G-10), which is useful as a light-emitting element material, can be obtained.

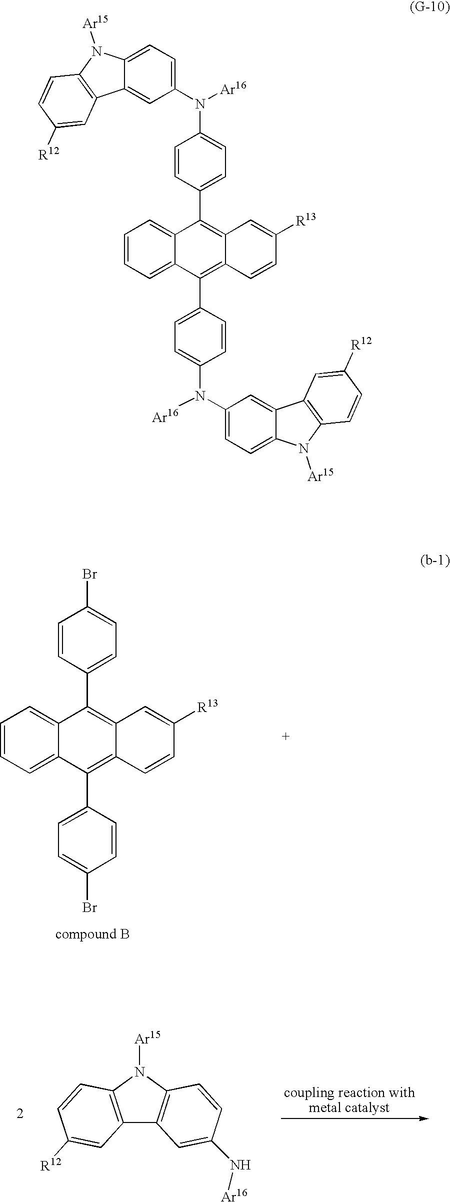

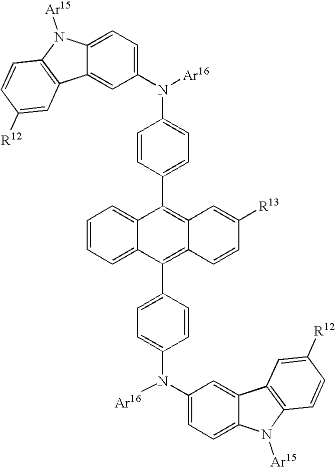

In the general formula (G-10) and the synthesis scheme (b-1), each of Ar¹⁵ and Ar¹⁶ represents an aryl group having 1 to 12 carbon atoms such as phenyl, biphenyl, or naphthyl. In addition, R¹² represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. R¹³ represents any one of hydrogen, methyl, and tert-butyl. It is to be noted that the aryl group may have a substituent or is not required to have a substituent.

The thus obtained anthracene derivative has resistance to repeated oxidation reactions, and can exhibit blue light emission. Therefore, the anthracene derivative is useful especially as a light-emitting element material serving as a light-emitting substance (also referred to as a guest). In addition, the anthracene derivative represented by the general formula (G-10) is highly suitable for being used in combination with an organic compound that is useful as a host of a light-emitting substance, which has an excellent electron transporting property and a large energy gap and exhibits blue light emission, such as 2-tert-butyl-9,10-di(2-naphthyl)anthracene (abbreviation: t-BuDNA), 9-[4-(N-carbazolyl)]phenyl-10-phenylanthracene (abbreviation: CZPA), 9-[4-(3,6-diphenyl-N-carbazolyl)]phenyl-10-phenylanthracene (abbreviation: DPCzPA), or diphenyl anthracene. By combining the anthracene derivative represented by the general formula (G-7) and t-BuDNA, CzPA, diphenyl anthracene, or the like to be used, a light-emitting element, which can moderately trap holes, prevent holes from going through from a light-emitting layer to other layers, and exhibit blue light emission with favorable chromaticity, can be manufactured.

Further, the compound B used in the synthesis scheme (b-1) is obtained by a synthesis as represented by the following synthesis scheme (c-1).

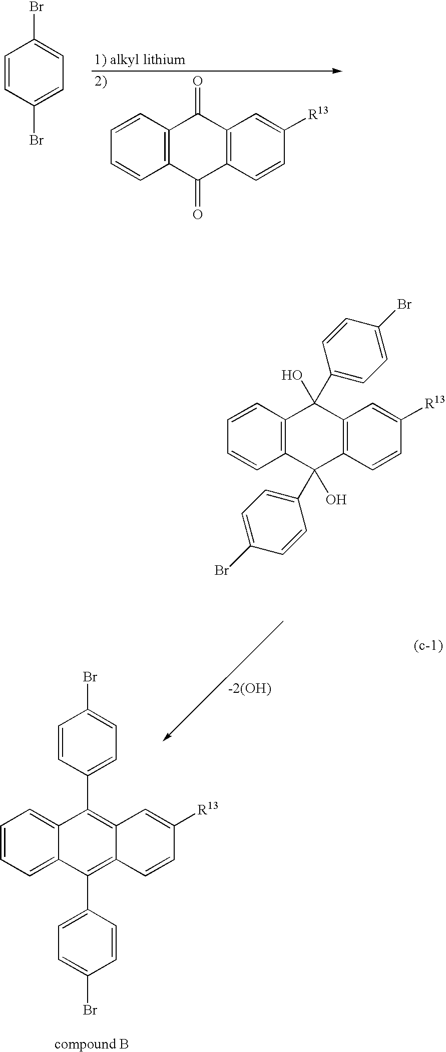

In the synthesis scheme (c-1), R¹³ represents any one of hydrogen, methyl, and tert-butyl.

### Embodiment Mode 3

One mode of a manufacturing method of a light-emitting element material using a carbazole derivative of the present invention will be explained.

As shown by the following synthesis scheme (d-1), coupling reaction of a carbazole derivative represented by a general formula (G-9) and a compound C having a structure in which three position of carbazole is substituted by a halogen group is performed by using a metal catalyst such as a palladium catalyst; accordingly, a carbazole derivative represented by the following general formula (G-11), which is useful as a light-emitting element material, can be obtained.

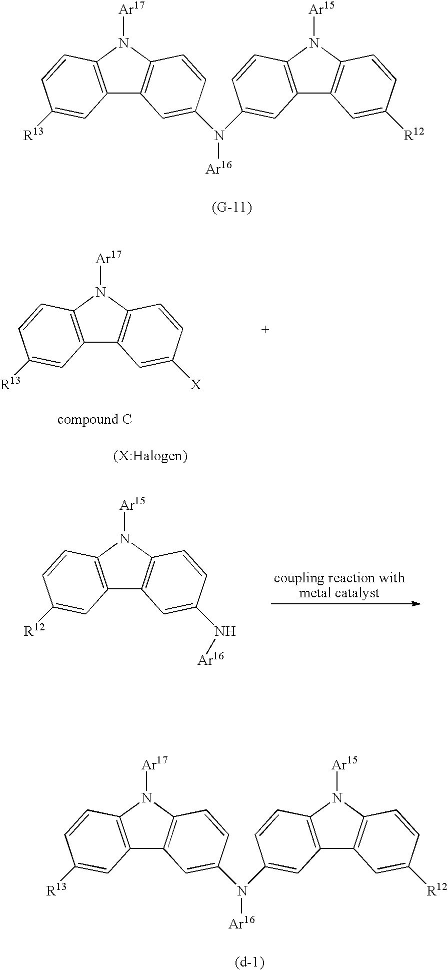

In the general formula (G-11) and a synthesis scheme (d-1), each of Ar¹⁵, Ar¹⁶, and Ar¹⁷ represents an aryl group having 1 to 12 carbon atoms such as phenyl, biphenyl, or naphthyl. In addition, each of R¹² and R¹³ represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that the aryl group may have a substituent, or is not required to have a substituent.

The carbazole derivative represented by the general formula (G-11) has resistance to repeated oxidation reactions. Ionization potential of the carbazole derivative represented by the general formula (G-11) has a small difference from a work function in a transparent electrode material such as indium tin oxide (ITO), indium zinc oxide (IZO), or tin oxide, which is used for forming an anode. In addition, the carbazole derivative represented by the general formula (G-11) has a favorable hole transporting property. Therefore, the carbazole derivative represented by the general formula (G-11) is useful especially as a hole injecting material which is used for forming a hole injecting layer among light-emitting element materials. It is to be noted that the carbazole derivative represented by the general formula (G-11) may be used as a light-emitting element material not only for forming a hole injecting layer but also for forming other layers.

### Embodiment Mode 4

One mode of a manufacturing method of a light-emitting element material using a carbazole derivative of the present invention will be explained.

As shown by the following synthesis scheme (e-1), coupling reaction of a carbazole derivative represented by a general formula (G-9) and a compound D having a structure in which three position and six position of carbazole are substituted by a halogen group is performed by using a metal catalyst such as a palladium catalyst; accordingly, a carbazole derivative represented by the following general formula (G-12), which is useful as a light-emitting element material, can be obtained.

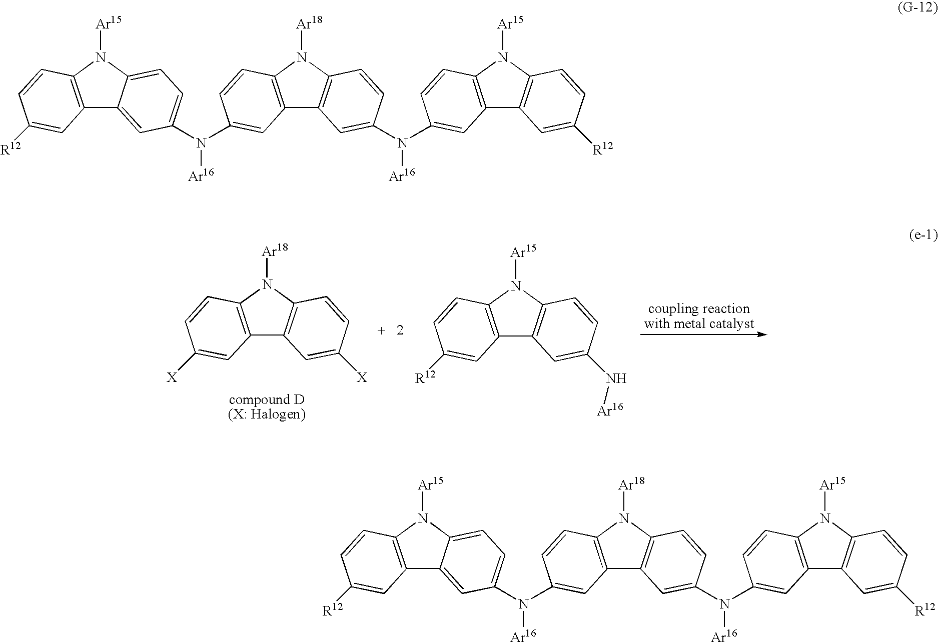

In the general formula (G-12) and the synthesis scheme (e-1), each of Ar¹⁵, Ar^{16,} and Ar¹⁸ represents an aryl group having 1 to 12 carbon atoms such as phenyl, biphenyl, or naphthyl. In addition, R¹² represents any one of hydrogen, an alkyl group having 1 to 4 carbon atoms such as methyl, ethyl, or tert-butyl, and an aryl group having 6 to 12 carbon atoms such as phenyl, biphenyl, or naphthyl. It is to be noted that the aryl group may have a substituent, or is not required to have a substituent.

The carbazole derivative represented by the general formula (G-12) has resistance to repeated oxidation reactions. Ionization potential of the carbazole derivative represented by the general formula (G-12) has a small difference from a work function in a transparent electrode material such as indium tin oxide (ITO), indium zinc oxide (IZO), or tin oxide, which is used for forming an anode. In addition, the carbazole derivative represented by the general formula (G-12) has a favorable hole transporting property. Therefore, the carbazole derivative represented by the general formula (G-12) is useful especially as a hole injecting material which is used for forming a hole injecting layer or a hole transporting material which is used for forming a hole transporting layer, among light-emitting element materials. It is to be noted that the carbazole derivative represented by the general formula (G-12) may be used as a light-emitting element material not only for forming a hole injecting layer or a hole transporting layer but also for forming other layers.

### Embodiment Mode 5

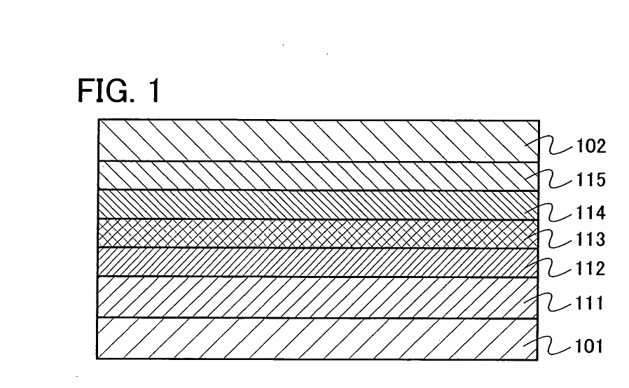

One mode of a light-emitting element using a light-emitting element material which is synthesized by using a carbazole derivative of the present invention will be explained with reference to FIG. 1.

FIG. 1 shows a light-emitting element including a light-emitting layer 113 between a first electrode 101 and a second electrode 102. In this embodiment mode, an anthracene derivative represented by a general formula (G-10) is contained in the light-emitting layer 113. In addition, in the light-emitting element of FIG. 1, a hole injecting layer 111 and a hole transporting layer 112 are sequentially stacked to be provided between the first electrode 101 and the light-emitting layer 113, and an electron transporting layer 114 and an electron injecting layer 115 are sequentially stacked to be provided between the second electrode 102 and the light-emitting layer 113.

In such a light-emitting element, holes injected from the first electrode 101 side and electrons injected from the second electrode 102 side are recombined in the light-emitting layer 113, and the anthracene derivative represented by the general formula (G-10) is in an excited state. The excited anthracene derivative emits light in returning to a ground state. As described above, the anthracene derivative represented by the general formula (G-10) serves as a light-emitting substance. In addition, the first electrode 101 and the second electrode 102 serve as an anode and a cathode, respectively.

Hereinafter, the first electrode 101, the second electrode 102, and each layer provided between the first electrode 101 and the second electrode 102 will be specifically explained.

The first electrode 101 and the second electrode 102 are not particularly limited, and gold (Au), platinum (Pt), nickel (Ni), tungsten (W), chromium (Cr), molybdenum (Mo), iron (Fe), cobalt (Co), copper (Cu), palladium (Pd), or the like, in addition to indium tin oxide (ITO), indium tin oxide containing silicon oxide, and indium oxide containing 2 to 20 wt % of zinc oxide, can be used for forming the first electrode 101 and the second electrode 102. Also, in addition to aluminum, alloy of magnesium and silver, alloy of aluminum and lithium, or the like can be used for forming the first electrode 101. Further, a formation method of the first electrode 101 and the second electrode 102 is not particularly limited, and a sputtering method, an evaporation method, or the like can be used. In order to extract emitted light to outside, it is preferable that one or both of the first electrode 101 and the second electrode 102 be formed using a transparent electrode material such as indium tin oxide, or silver, aluminum, or the like to be several nm to several tens nm thick so that visible light can be transmitted.

The hole injecting layer 111 has a function of helping injection of holes from the first electrode 101 to the hole transporting layer 112. By providing the hole injecting layer 111, a difference in ionization potential between the first electrode 101 and the hole transporting layer 112 is reduced, and holes can be easily injected. It is preferable that the hole injecting layer 111 be formed of a substance of which the ionizing potential is lower than that of a substance contained in the hole transporting layer 112 and of which ionization potential is higher than that of a substance contained in the first electrode 101, or a substance in which an energy band is bent when provided as a thin film with a thickness of 1 to 2 nm between the hole transporting layer 112 and the first electrode 101. As a specific example of a substance which can be used for forming the hole injecting layer 111, the following can be given: a phthalocyanine compound such as phthalocyanine (H₂Pc) or copper phthalocyanine (CuPc), a high molecular compound such as poly (ethylene dioxythiophene)/poly(styrene sulfonate), or the like. In other words, the hole injecting layer 111 can be formed by selecting a substance of which ionization potential in the hole injecting layer 111 is relatively lower than that in the hole transporting layer 112 from hole transporting substances. Further, it is preferable that the first electrode 101 be formed using a substance having a high work function such as indium tin oxide, in a case of providing the hole injecting layer 111.

The hole transporting layer 112 has a function of transporting holes injected from the first electrode 101 side to the light-emitting layer 113. As described above, by providing the hole transporting layer 112, a distance between the first electrode 101 and the light-emitting layer 113 can be increased. As a result, quenching of light emission due to metal contained in the first electrode 101 or the like can be prevented. It is preferable that the hole transporting layer be formed using a hole transporting substance, especially a substance having hole mobility of 1×10⁶ cm²/Vs or more. It is to be noted that the hole transporting substance is a substance of which hole mobility is higher than electron mobility and a value of a ratio of hole mobility with respect to electron mobility (=hole mobility/electron mobility) is preferably larger than 100. As a specific substance which can be used for forming the hole transporting layer 112, the following can be given: 4,4′-bis[N-(1-napthyl)-N-phenylamino]biphenyl (abbreviation: NPB), 4,4′-bis[N-(3-methylphenyl)-N-phenylamino]biphenyl (abbreviation: TPD), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis{N-[4-(N,N-di-m-tolylamino)phenyl]-N-phenylamino}biphenyl (abbreviation: DNTPD), 1,3,5-tris[N,N-di(m-tolyl)amino]benzene (abbreviation: m-MTDAB), 4,4′,4″-tris(N-carbazolyl)triphenylamine (abbreviation: TCTA), phthalocyanine (H₂Pc), copper phthalocyanine (CuPc), vanadyl phthalocyanine (VOPc), 4,4′-bis[N-(4-biphenylyl)-N-phenylamino]biphenyl (abbreviation: BBPB), or the like. It is to be noted that it is more preferable that the hole transporting layer 112 be formed by selecting especially a substance of which an energy gap is larger than that of a substance which is used as a host among hole transporting substances. In addition, the hole transporting layer 112 may have a multilayer structure in which two or more of the layers formed using the above described material are combined.

The light-emitting layer 113 is preferably a layer in which the anthracene derivative represented by the general formula (G-10) is dispersed and included in a layer containing, as its main component, a substance (referred to as a host) having a larger energy gap than the anthracene derivative and higher ionization potential than the anthracene derivative. Accordingly, quenching of light emission from the anthracene derivative due to concentration of the anthracene derivative itself can be prevented. It is to be noted that the energy gap refers to energy gap between a LUMO (Lowest Unoccupied Molecular Orbital) level and a HOMO (Highest Occupied Molecular Orbital) level.

More specifically, a substance used as a host preferably has ionization potential higher than 5.3 eV and an energy gap larger than 2.8 eV, and is a substance having an electron transporting property which is higher than a hole transporting property. As such a substance, for example, an anthracene derivative such as t-BuDNA, CzPA, or diphenyl anthracene; a phenanthroline derivative such as BCP; an oxadiazole derivative, or a triazine derivative can be given. One or two or more of these substances may be selected to be mixed so that the anthracene derivative represented by the general formula (G-10) is in a dispersion state. By making the light-emitting layer 113 have such a structure, holes can be efficiently trapped in the anthracene derivative represented by the general formula (G-10). As a result, a light-emitting element having favorable light emission efficiency can be obtained. Moreover, the electron transporting layer 114 is formed of a substance having a small energy gap in many cases, and excited energy easily moves from the light-emitting layer 113; however, by making the light-emitting layer 113 have the above-described structure, a recombination region (a light-emitting region) of holes and electrons in the light-emitting layer 113 is formed on the hole transporting layer 112 side, and the excited energy can be prevented from moving to the electron transporting layer 114. As a result, chromaticity can be prevented from deteriorating due to light emission generated in a different layer from the light-emitting layer 113. Further, a layer in which a plurality of compounds is mixed as in the light-emitting layer 113 can be formed by a co-evaporation method. Here, a co-evaporation method refers to an evaporation method by which each raw material is vaporized from each of a plurality of evaporation sources provided in one processing chamber and the vaporized raw materials are mixed in a gaseous state to be deposited on an object to be processed.

The electron transporting layer 114 has a function of transporting electrons injected from the second electrode 102 to the light-emitting layer 113. By providing the electron transporting layer 114, a distance between the second electrode 102 and the light-emitting layer 113 can be increased. As a result, quenching of light emission due to metal contained in the second electrode 102 or the like can be prevented. It is preferable that the electron transporting layer 114 be formed using an electron transporting substance, especially a substance having electron mobility of 1×10⁻⁶ cm²/Vs or more. It is to be noted that the electron transporting substance is a substance of which electron mobility is higher than hole mobility and a value of a ratio of electron mobility with respect to hole mobility (=electron mobility/hole mobility) is larger than 100. As a specific substance which can be used for forming the electron transporting layer 114, the following can be given: 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproin (abbreviation: BCP), 4,4-bis(5-methylbenzoxazol-2-yl)stilbene (abbreviation: BzOs), or the like, in addition to a metal complex such as tris(8-quinolinolato)aluminum (abbreviation: Alq₃), tris(4-methyl-8-quinolinolato)aluminum (abbreviation: Almq₃), bis(10-hydroxybenzo[h]-quinolinato)beryllium (abbreviation: BeBq₂), bis(2-methyl-8-quinolinolato)-4-phenylphenolato-aluminum (abbreviation: BAlq), bis[2-(2-hydroxyphenyl)benzoxazolate]zinc (abbreviation: Zn(BOX)₂), or bis[2-(2-hydroxyphenyl)benzothiazolate]zinc (abbreviation: Zn(BTZ)₂). It is to be noted that it is more preferable that the electron transporting layer 114 be formed by selecting especially a substance of which an energy gap is larger than that of a substance which is used as a host among electron transporting substances. In addition, the electron transporting layer 114 may have a multilayer structure in which two or more of the layers formed using the above described material are combined.

The electron injecting layer 115 has a function of helping injection of electrons from the second electrode 102 to the electron transporting layer 114. The electron injecting layer 115 can be formed using a substance having relatively higher electron affinity than that of a substance used for forming the electron transporting layer 114, which is selected from substances that can be used for forming the electron transporting layer 114, such as BPhen, BCP, p-EtTAZ, TAZ, and BzOs. By forming the electron injecting layer 115 as described above, a difference in electron affinity between the second electrode 102 and the electron transporting layer 114 is reduced, and electrons are easily injected. In addition, the electron injecting layer 115 may contain an inorganic substance such as alkali metal such as Li or Cs; oxide of alkali metal such as lithium oxide (Li₂O), kalium oxide (K₂O), sodium oxide (Na₂O); oxide of alkaline earth metal such as calcium oxide (CaO) or magnesium oxide (MaO); fluoride of alkali metal such as lithium fluoride (LiF) or cesium fluoride (CsF); fluoride of alkaline earth metal such as calcium fluoride (CaF₂); or alkaline earth metal such as Mg or Ca. In addition, the electron injecting layer 115 may have a structure including the organic substance as described above or may have a structure including an inorganic substance such as fluoride of alkali metal such as LiF or fluoride of alkaline earth metal such as CaF₂. By providing the electron injecting layer 115 as a thin film having a thickness of 1 to 2 nm by using an inorganic substance such as fluoride of alkali metal such as LiF or fluoride of alkaline earth metal such as CaF₂, an energy band of the electron injecting layer 115 is bent, or tunnel current flows; accordingly, electrons are easily injected from the second electrode 102 to the electron transporting layer 114.

It is to be noted that a hole generating layer may be provided instead of the hole injecting layer 111, or an electron generating layer may be provided instead of the electron injecting layer 115.

Here, the hole generating layer generates holes. The hole generating layer can be formed by mixing at least one substance selected from hole transporting substances and a substance showing an electron accepting property with respect to the hole transporting substance. Here, as the hole transporting substance, the similar substance to the substance which can be used for forming the hole transporting layer 112 can be used. As the substance showing an electron accepting property, it is preferable to use metal oxide such as molybdenum oxide, vanadium oxide, ruthenium oxide, or rhenium oxide.

The electron generating layer generates electrons. The electron generating layer can be formed by mixing at least one substance selected from electron transporting substances and a substance showing an electron donating property with respect to the electron transporting substance. Here, as the electron transporting substance, the similar substance to the substance which can be used for forming the electron transporting layer 114 can be used. As the substance showing an electron donating property, a substance selected from alkali metal and alkaline earth metal, specifically lithium (Li), calcium (Ca), sodium (Na), kalium (K), magnesium (Mg), or the like can be used.

The light-emitting element of the above-described mode can be formed by a manufacturing method by which, after the first electrode 101 is formed, the hole injecting layer 111, the hole transporting layer 112, the light-emitting layer 113, the electron transporting layer 114, and the electron injecting layer 115 are sequentially stacked thereover and the second electrode 102 is formed, or a manufacturing method by which, after the second electrode 102 is formed, the electron injecting layer 115, the electron transporting layer 114, the light-emitting layer 113, the hole transporting layer 112, and the hole injecting layer 111 are sequentially stacked thereover and the first electrode 101 is formed. It is to be noted that each of the hole injecting layer 111, the hole transporting layer 112, the light-emitting layer 113, the electron transporting layer 114, and the electron injecting layer 115 may be formed by any of an evaporation method, an ink jet method, an application method, and the like. In addition, the first electrode 101 or the second electrode 102 may be formed by any of a sputtering method, an evaporation method, and the like.

The light-emitting element of the present invention having the above-described structure is manufactured by using a compound containing an anthracene skeleton and an amine skeleton such as the anthracene derivative of the present invention; therefore, there are a few changes of characteristics of the light-emitting element in accordance with a change of characteristics of a light-emitting substance due to repeated oxidation reactions, and stable light emission can be displayed for a long time. In addition, the light-emitting element of the present invention is formed using the anthracene derivative of the present invention; therefore, blue light emission with favorable chromaticity can be exhibited.

### Embodiment Mode 6

One mode of a light-emitting element using a light-emitting element material which is synthesized by using a carbazole derivative of the present invention will be explained. A light-emitting element explained in this embodiment mode is similar to the light-emitting element described in Embodiment Mode 5 in terms of sequentially providing a hole injecting layer, a hole transporting layer, a light-emitting layer, an electron transporting layer, and an electron injecting layer between electrodes; therefore, the light-emitting element of this embodiment mode will be also explained with reference to FIG. 1 which is used for explaining Embodiment Mode 5.

FIG. 1 shows a light-emitting element having a light-emitting layer 113 between a first electrode 101 and a second electrode 102. In the light-emitting element of FIG. 1, a hole injecting layer 111 and a hole transporting layer 112 are sequentially stacked between the first electrode 101 and the light-emitting layer 113, and an electron transporting layer 114 and an electron injecting layer 115 are sequentially stacked between the second electrode 102 and the light-emitting layer 113. In addition, the first electrode 101 and the second electrode 102 serve as an anode and a cathode, respectively.

In such a light-emitting element, holes injected from the first electrode 101 side and electrons injected from the second electrode 102 side are recombined in the light-emitting layer 113, and a light-emitting substance contained in the light-emitting layer 113 is in an excited state. The excited light-emitting substance emits light in returning to a ground state. Here, the light-emitting substance refers to a substance which exhibits a desired light emission color when the light-emitting element is driven.

Hereinafter, the first electrode 101 and the second electrode 102 are the same as those in Embodiment Mode 5; therefore, the explanation is omitted in this embodiment mode.

The hole injecting layer 111 has a function of helping injection of holes from the first electrode 101 to the hole transporting layer 112. In this embodiment mode, the hole injecting layer 111 is formed by using a carbazole derivative represented by a general formula (G-11) or (G-12). By providing the hole injecting layer 111 formed by using the carbazole derivative represented by the general formula (G-11) or (G-12) as described above, a difference in ionization potential between the first electrode 101 and the hole transporting layer 112 is reduced, and holes can be easily injected to the hole transporting layer 112.

The hole transporting layer 112 is the same as the one in Embodiment Mode 5; therefore, the explanation is omitted in this embodiment mode.

The light-emitting layer 113 may have the similar structure to the one explained in Embodiment Mode 5 or may have a different structure, and a case where the light-emitting layer 113 has a different structure from the one described in Embodiment mode 5 is explained. The light-emitting layer 113 contains a light-emitting substance. The light-emitting layer 113 may be a layer formed of only a light-emitting substance; however, the light-emitting layer 113 is preferably a layer in which a light-emitting substance is mixed so as to be dispersed in a layer containing, as its main component, a substance having larger energy gap than that of the light-emitting substance in a case where concentration quenching is generated. By dispersing the light-emitting substance to be contained in the light-emitting layer 113, quenching of light emission due to the concentration can be prevented. Here, the energy gap refers to an energy gap between a LUMO level and a HOMO level.

The light-emitting substance is not particularly limited, and a substance which can emit light with favorable light emission efficiency and a desired light emission wavelength may be used. For example, when red light emission is desired to be obtained, a substance which exhibits light emission having a peak of an emission spectrum in a wavelength range of 600 to 680 nm, such as
4-dicyanomethylene-2-isopropyl-6-[2-(1,1,7,7-tetramethyl-9-julolidyl)ethenyl]-4H-pyran (abbreviation: DCJTI),
4-dicyanomethylene-2-methyl-6-[2-(1,1,7,7-tetramethyl-9-julolidyn)ethenyl]-4H-pyran (abbreviation: DCJT),
4-dicyanomethylene-2-tert-butyl-6-[2-(1,1,7,7-tetramethyl-9-julolidyn)ethenyl]-4H-pyran (abbreviation: DCJTB), periflanthene, or
2,5-dicyano-1,4-bis[2-(10-methoxy-1,1,7,7-tetramethyl-9-julolidyl)ethenyl]benzene can be used as the light-emitting substance. In addition, when green light emission is desired to be obtained, a substance which exhibits light emission having a peak of an emission spectrum in a wavelength range of 500 to 550 nm, such as N,N′-dimethylquinacridone (abbreviation: DMQd), coumarin 6, coumarin 545T, or tris(8-quinolinolato)aluminum (abbreviation: Alq₃) can be used as the light-emitting substance. Moreover, when blue light emission is desired to be obtained, a substance which exhibits light emission having a peak of an emission spectrum in a wavelength range of 420 to 500 nm, such as 9,10-bis(2-naphthyl)-tert-butylanthracene (abbreviation: t-BuDNA), 9,9′-bianthryl, 9,10-diphenylanthracene (abbreviation: DPA), 9,10-bis(2-naphthyl)anthracene (abbreviation: DNA), bis(2-methyl-8-quinolinolato)-4-phenylphenolato-gallium (abbreviation: BGaq), or bis(2-methyl-8-quinolinolato)-4-phenylphenolato-aluminum (abbreviation: BAlq) can be used as the light-emitting substance. In addition to the above-described substance which emits fluorescence light, a substance which emits phosphorescence light, such as bis[2-(3,5-bis(trifluoromethyl)phenyl)pyridinato-N,C^{2′}]iridium(III)picolinate (abbreviation: Ir(CF₃ppy)₂(pic)), bis[2-(4,6-difluorophenyl)pyridinato-N,C^{2′}]iridium(III)acetylacetonate (abbreviation: FIr(acac)), bis[2-(4,6-difluorophenyl)pyridinato-N,C^{2′}]iridium(III)picolinate (abbreviation: FIr(pic)), tris(2-phenylpyridinato-N,C^{2′})iridium (abbreviation: Ir(ppy)₃), can also be used as the light-emitting substance.

A substance which is contained in the light-emitting layer 113 with the light-emitting substance and used for making the light-emitting substance in a dispersion state is not particularly limited, and the substance may be appropriately selected in view of an energy gap or the like of the substance used as the light-emitting substance. For example, a metal complex or the like such as bis[2-(2-hydroxyphenyl)pyridinato]zinc (abbreviation: Znpp₂) or bis[2-(2-hydroxyphenyl)benzoxazolato]zinc (abbreviation: ZnBOX) can be used with the light-emitting substance, in addition to an anthracene derivative such as 9,10-di(2-naphthyl)-2-tert-butylanthracene (abbreviation: t-BuDNA); a carbazole derivative such as 4,4′-bis(N-carbazolyl)biphenyl (abbreviation: CBP); and a quinoxaline derivative such as 2,3-bis(4-diphenylaminophenyl)quinoxaline (abbreviation: TPAQn) or 2,3-bis{4-[N(1-naphthyl)-N-phenylamino]phenyl}-dibenzo[f,h]quinoxaline (abbreviation: NPADiBzQn).

It is to be noted that a layer in which a plurality of compounds is mixed as in the light-emitting layer 113 can be formed by a co-evaporation method. Here, a co-evaporation method refers to an evaporation method by which each raw material is vaporized from a plurality of evaporation sources provided in one processing chamber and the vaporized raw materials are mixed in a gaseous state to be deposited on an object to be processed.

The electron transporting layer 114 and the electron injecting layer 115 is similar to those in Embodiment Mode 5; therefore, the description is omitted in this embodiment mode.

The light-emitting element of the above-described mode can be formed by a manufacturing method by which, after the first electrode 101 is formed, the hole injecting layer 111, the hole transporting layer 112, the light-emitting layer 113, the electron transporting layer 114, and the electron injecting layer 115 are sequentially stacked thereover and the second electrode 102 is formed, or a manufacturing method by which, after the second electrode 102 is formed, the electron injecting layer 115, the electron transporting layer 114, the light-emitting layer 113, the hole transporting layer 112, and the hole injecting layer 111 are sequentially stacked thereover and the first electrode 101 is formed. It is to be noted that each of the hole injecting layer 111, the hole transporting layer 112, the light-emitting layer 113, the electron transporting layer 114, and the electron injecting layer 115 may be formed by any of an evaporation method, an ink jet method, an application method, and the like. In addition, the first electrode 101 or the second electrode 102 may be formed by any of a sputtering method, an evaporation method, and the like.

The light-emitting element of the present invention having the above-described structure is formed by using the carbazole derivative represented by the general formula (G-11) or (G-12); therefore, there are a few changes of characteristics of the light-emitting element in accordance with a change of characteristics of a light-emitting substance due to repeated oxidation reactions, and stable light emission can be exhibited for a long time.

It is to be noted that an application of the light-emitting element material represented by the general formulas (G-10) to (G-12) is not limited to formation of the light-emitting layer or the hole injecting layer as described in Embodiment Modes 5 and 6, and may be used for forming the hole transporting layer or the hole generating layer, for example.

### Embodiment Mode 7

One mode of a light-emitting element using a light-emitting element material synthesized by using a carbazole drivative of the present invention will be explained. A light-emitting element explained in this embodiment mode is similar to the light-emitting element described in Embodiment Mode 5 in terms of sequentially providing a hole injecting layer, a hole transporting layer, a light-emitting layer, an electron transporting layer, and an electron injecting layer between electrodes; therefore, the light-emitting element of this embodiment mode will be explained with reference to FIG. 1 used for explaining Embodiment Mode 5.

FIG. 1 shows a light-emitting element having a light-emitting layer 113 between a first electrode 101 and a second electrode 102. In the light-emitting element of FIG. 1, a hole injecting layer 111 and a hole transporting layer 112 are sequentially stacked between the first electrode 101 and the light-emitting layer 113, and an electron transporting layer 114 and an electron injecting layer 115 are sequentially stacked between the second electrode 102 and the light-emitting layer 113. In addition, the first electrode 101 and the second electrode 102 serve as an anode and a cathode, respectively.

In such a light-emitting element, holes injected from the first electrode 101 side and electrons injected from the second electrode 102 side are recombined in the light-emitting layer 113, and a light-emitting substance contained in the light-emitting layer 113 is in an excited state. The excited light-emitting substance emits light in returning to a ground state. Here, the light-emitting substance refers to a substance which exhibits a desired light emission color when the light-emitting element is driven.

Hereinafter, the first electrode 101 and the second electrode 102 are the same as those in Embodiment Mode 5; therefore, the explanation is omitted in this embodiment mode.

The hole injecting layer 111 is similar to the one in Embodiment Mode 5; therefore, the explanation is omitted in this Embodiment Mode.

The hole transporting layer 112 has a function of transporting holes injected from the first electrode 101 side to the light-emitting layer 113. In this embodiment mode, the hole transporting layer 112 is formed using a carbazole derivative represented by a general formula (G-11) or (G-12). By providing the hole transporting layer 112 formed using the carbazole derivative represented by the general formula (G-11) or (G-12), holes injected from the first electrode 101 side can be efficiently transported to the light-emitting layer 113, and excited energy can be prevented from moving to other layers from the light-emitting layer 113.

The light-emitting layer 113 is similar to the one described in Embodiment Mode 5 or 6; therefore, the description is omitted in this embodiment mode.

The electron transporting layer 114 and the electron injecting layer 115 are similar to those described in Embodiment Mode 5; therefore, the description is omitted in this embodiment mode.

The light-emitting element of the above-described mode can be formed by a manufacturing method by which, after the first electrode 101 is formed, the hole injecting layer 111, the hole transporting layer 112, the light-emitting layer 113, the electron transporting layer 114, and the electron injecting layer 115 are sequentially stacked thereover and the second electrode 102 is formed, or a manufacturing method by which, after the second electrode 102 is formed, the electron injecting layer 115, the electron transporting layer 114, the light-emitting layer 113, the hole transporting layer 112, and the hole injecting layer 111 are sequentially stacked thereover and the first electrode 101 is formed. It is to be noted that each of the hole injecting layer 111, the hole transporting layer 112, the light-emitting layer 113, the electron transporting layer 114, and the electron injecting layer 115 may be formed by any of an evaporation method, an ink jet method, an application method, and the like. In addition, the first electrode 101 or the second electrode 102 may be formed by any of a sputtering method, an evaporation method, and the like.

The light-emitting element of the present invention having the above-described structure is formed by using the carbazole derivative represented by the general formula (G-11) or (G-12); therefore, there are a few changes of characteristics of the light-emitting element in accordance with a change of characteristics of a light-emitting substance due to repeated oxidation reactions, and stable light emission can be exhibited for a long time.

### Embodiment Mode 8

Each of the light-emitting elements of the present invention described in Embodiment Modes 5 to 7 has resistance to repeated oxidation reactions and can emit light in a favorable state for a long time. Therefore, by using the light-emitting element of the present invention, a light-emitting device which can provide a favorable display image or the like for a long time can be obtained. In addition, the light-emitting element of the present invention explained in Embodiment Mode 4 can exhibit blue light emission with favorable chromaticity. Therefore, by using the light-emitting element of the present invention, a light-emitting device which exhibits blue light emission with favorable chromaticity and displays an image having excellent colors can be obtained.

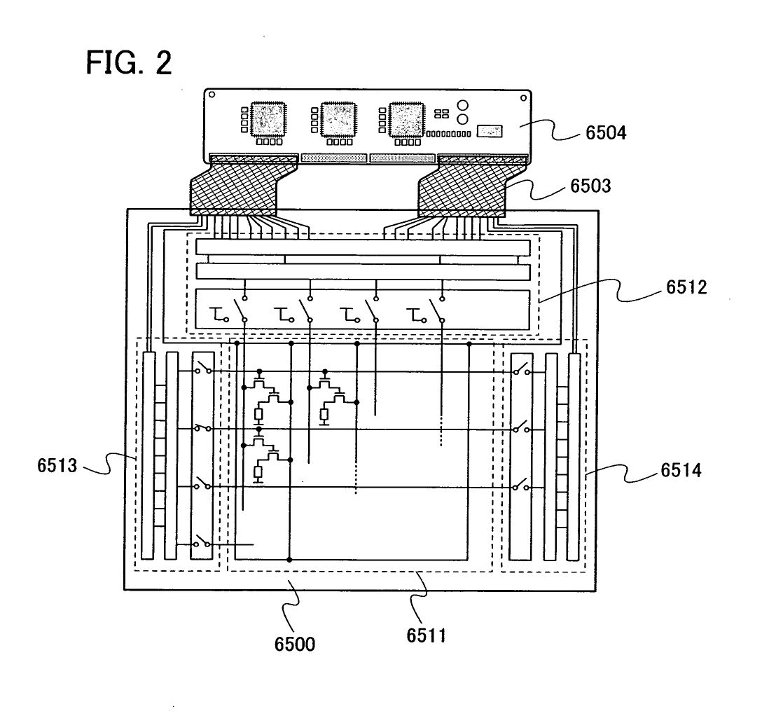

In this embodiment mode, a circuit structure and a driving method of a light-emitting device having a display function will be explained with reference to FIGS. 2 to 5.

FIG. 2 is a top view schematically showing a light-emitting device to which the present invention is applied. In FIG. 2, a pixel portion 6511, a source signal line driver circuit 6512, a writing gate signal line driver circuit 6513, and an erasing gate signal line driver circuit 6514 are provided over a substrate 6500. Each of the source signal line driver circuit 6512, the writing gate signal line driver circuit 6513, and the erasing gate signal line driver circuit 6514 is connected to an FPCs (flexible printed circuits) 6503 that are external input terminals through a group of wirings. Each of the source signal line driver circuit 6512, the writing gate signal line driver circuit 6513, and the erasing gate signal line driver circuit 6514 receives a video signal, a clock signal, a start signal, a reset signal, or the like from the FPCs 6503. In addition, a printed wiring board (PWB) 6504 is attached to the FPCs 6503. It is to be noted that a driver circuit portion is not necessarily provided over the same substrate as the pixel portion 6511. For example, the driver circuit portion may be provided outside the substrate by using a TCP (Tape Carrier Package) in which an IC chip is mounted over an FPC where a wiring pattern is formed, or the like.

In the pixel portion 6511, a plurality of source signal lines extending in columns are arranged in rows. In addition, a current supply lines are arranged in rows. A plurality of gate signal lines extending in rows are arranged in columns in the pixel portion 6511. Moreover, a plurality of pairs of circuits each including a light-emitting element are arranged in the pixel portion 6511.

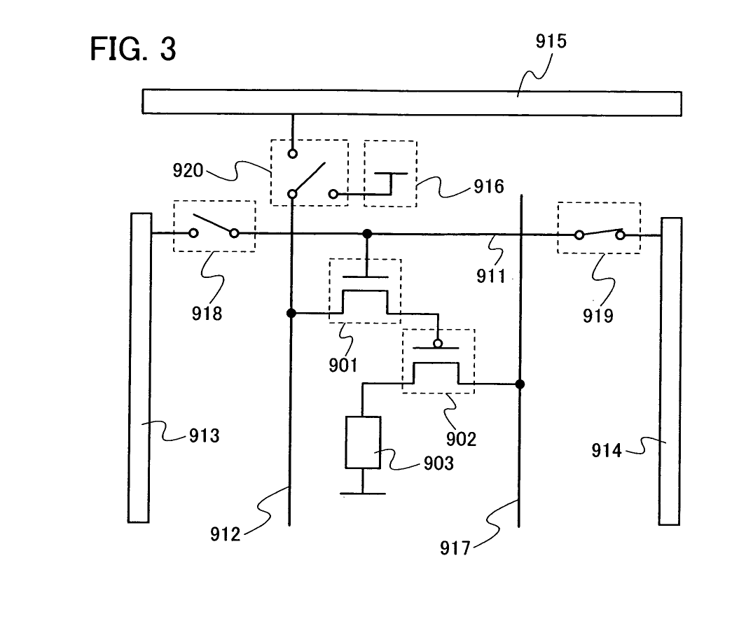

FIG. 3 shows a circuit for operating one pixel. The circuit shown in FIG. 3 includes a first transistor 901, a second transistor 902, and a light-emitting element 903.

Each of the first transistor 901 and the second transistor 902 is a three-terminal element including a gate electrode, a drain region, and a source region, and includes a channel region between the drain region and the source region. Here, since a source region and a drain region are switched with each other in accordance with a structure or operating conditions of a transistor, it is difficult to identify which one is the drain region or the source region. Therefore, in this embodiment mode, regions that serve as a source or a drain are referred to as a first electrode and a second electrode, respectively.

A gate signal line 911 and a writing gate signal line driver circuit 913 are provided so as to be electrically connected or disconnected by a switch 918. The gate signal line 911 and an erasing gate signal line driver circuit 914 are provided so as to be electrically connected or disconnected by a switch 919. In addition, a source signal line 912 is provided so as to be electrically connected to any of a source signal line driver circuit 915 and a power source 916 by a switch 920. A gate of the first transistor 901 is electrically connected to the gate signal line 911, a first electrode of the first transistor 901 is electrically connected to the source signal line 912, and a second electrode is electrically connected to a gate electrode of the second transistor 902. A first electrode of the second transistor 902 is electrically connected to a current supply line 917 and a second electrode is electrically connected to one electrode included in the light-emitting element 903. It is to be noted that the switch 918 may be included in the writing gate signal line driver circuit 913, the switch 919 may be included in the erasing gate signal line driver circuit 914, and the switch 920 may be included in the source signal line driver circuit 915.

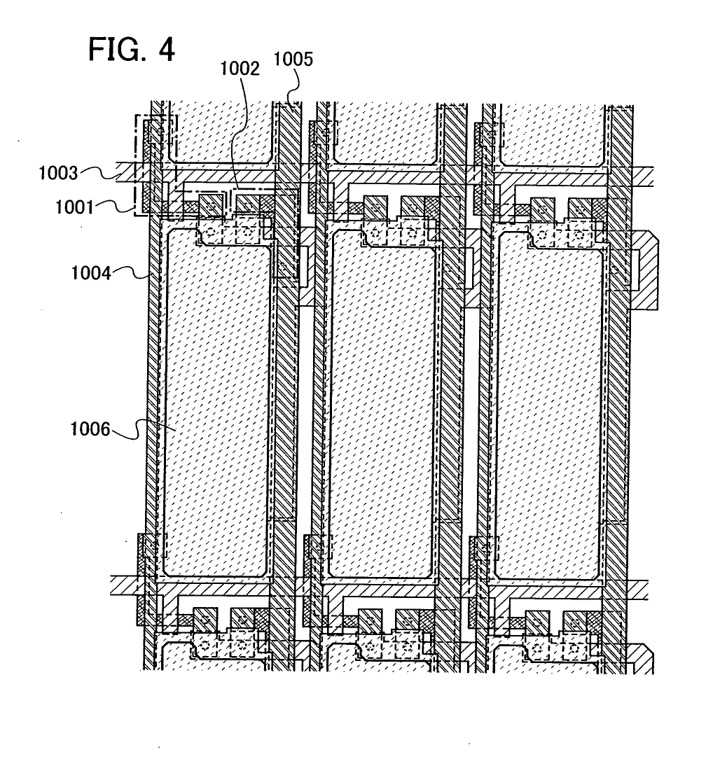

In addition, arrangement of a transistor, a light-emitting element, or the like in a pixel portion is not particularly limited. For example, arrangement shown in a top view of FIG. 4 can be employed. In FIG. 4, a first electrode of a first transistor 1001 is connected to a source signal line 1004 and a second electrode is connected to a gate electrode of a second transistor 1002. Moreover, a first electrode of the second transistor 1002 is connected to a current supply line 1005 and a second electrode is connected to an electrode 1006 of a light-emitting element. Part of a gate signal line 1003 serves as a gate electrode of the first transistor 1001.

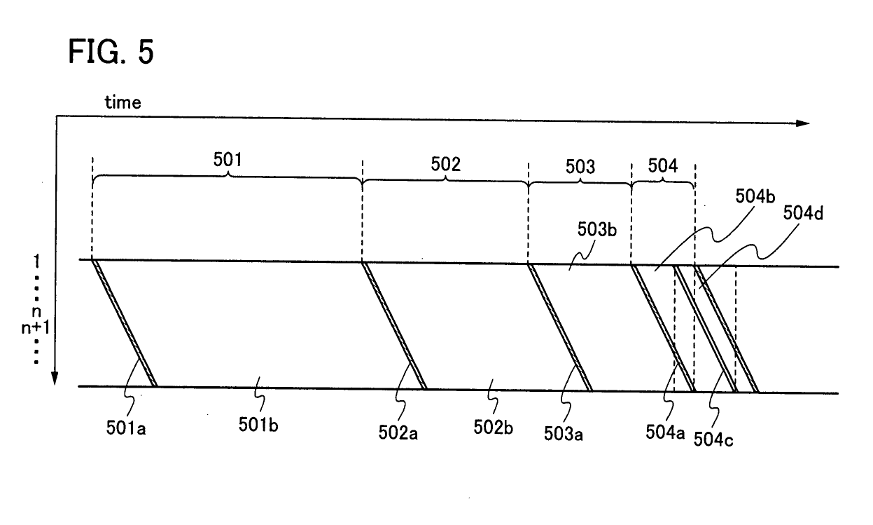

Next, a driving method will be explained. FIG. 5 is a view illustrating a frame operation in accordance with passage of time. In FIG. 5, the horizontal direction indicates passage of time, and the vertical direction indicates the number of scanning stages of gate signal lines.

When a light-emitting device of the present invention is used to display images, a rewrite operation and a display operation for a screen are repeated in a display period. Although the number of rewrites is not particularly limited, it is preferable that the number of rewrites be at least about 60 times per second so as not to make a viewer notice flickers. Here, a period for which a rewrite operation and a display operation are performed for one screen (one frame) is referred to as one frame period.

As shown in FIG. 5, one frame period is divided into four sub-frames 501, 502, 503, and 504 including writing periods 501a, 502a, 503a, and 504a and retention periods 501b, 502b, 503b, and 504b, respectively. A light-emitting element to which a signal for emitting light is given is made to be in an emitting state in a retention period. The ratio of the length of the retention period in each sub-frame is, the first sub-frame 501: the second sub-frame 502: the third sub-frame 503: the fourth sub-frame 504=2³: 2²: 2¹: 2⁰=8:4:2:1. This makes 4-bit gradation possible. However, the number of bits and the number of gradations are not limited to the ones described here. For example, eight sub-frames may be provided so as to perform 8-bit gradation.

An operation in one frame period will be explained. First, in the sub-frame 501, writing operations are sequentially performed for a first row to a last row. Consequently, the starting time of writing period is different depending on the rows. In rows for which the writing period 501a is completed, the state is shifted sequentially into the retention period 501b. In the retention periods, a light-emitting element to which a signal for emitting light is given is made to be in an emitting state. In addition, in rows for which the retention period 501b is completed, the state is shifted sequentially into the next sub-frame 502, and writing operations are sequentially performed for the first row to the last row as in the case of the sub-frame 501. Such operations as described above are repeated until the retention period 504b of the sub-frame 504 is completed. When the operation in the sub-frame 504 is completed, the next frame begins. Thus, a total of the time for which light is emitted in each sub-frame is emission time for each light-emitting element in one frame. By varying this emission time for each light-emitting element to have various combinations in one pixel, various display colors with different luminosity and chromaticity can be made.

As in the sub-frame 504, when forcible termination of a retention period of a row for which writing has been already completed and which is moved into the retention period is required before writing for the last row is completed, it is preferable that an erasing period 504c be provided after the retention period 504b and a row be controlled so as to be in a non-emission state forcibly. Then, the row forcibly made to be in the non-emitting state is kept in the non-emission state for a certain period (this period is referred to as a non-emission period 504d). Then, immediately after the writing period of the last row is completed, the state is shifted sequentially into the writing period (or the next frame), starting from the first row. This makes it possible to prevent the writing period of the sub-frame 504 from overlapping with the writing period of the next sub-frame.

Although the sub-frames 501 to 504 are arranged in the order from the longest retention period to the shortest in this embodiment mode, the arrangement as in this embodiment mode is not always required. For example, the sub-frames 501 to 504 may be arranged in the order from the shortest retention period to the longest, or may be arranged in a random order. In addition, the sub-frames may be further divided into a plurality of frames. In other words, scanning of the gate signal lines may be performed plural times while giving the same video signal.

Now, an operation of the circuit shown in FIG. 3 in a writing period and an erasing period will be explained.

First, an operation in a writing period will be explained. In the writing period, the gate signal line 911 in n-th row (n is a natural number) is electrically connected to the writing gate signal line driver circuit 913 through the switch 918, and disconnected to the erasing gate signal line driver circuit 914. In addition, the source signal line 912 is electrically connected to the source signal line driver circuit through the switch 920. Here, a signal is inputted to the gate of the first transistor 901 connected to the gate signal line 911 in n-th row (n is a natural number) to turn on the first transistor 901. Then, at this time, video signals are inputted at the same time into the source signal lines 912 in the first to the last columns. It is to be noted that the video signals inputted from the source signal lines 912 to the respective columns are independent of each other. The video signal inputted from the source signal line 912 is inputted to the gate electrode of the second transistor 902 through the first transistor 901 connected to each of the source signal lines 912. At this time, whether the light-emitting element 903 emits light or not is determined depending on the signal inputted to the second transistor 902. For example, when the second transistor 902 is a p-channel transistor, the light-emitting element 903 emits light by inputting a Low Level signal to the gate electrode of the second transistor 902. On the other hand, when the second transistor 902 is an n-channel transistor, the light-emitting element 903 emits light by inputting a High Level signal to the gate electrode of the second transistor 902.

Next, an operation in an erasing period will be explained. In the erasing period, the gate signal line 911 in n-th row (n is a natural number) is electrically connected to the erasing gate signal line driver circuit 914 through the switch 919 and disconnected to the writing gate signal line driver circuit 913. In addition, the source signal line 912 is electrically connected to the power source 916 through the switch 920. Here, a signal is inputted to the gate of the first transistor 901 connected to the gate signal line 911 in n-th row to turn on the first transistor 901. Then, at this time, erasing signals are inputted at the same time to the source signal lines 912 in the first to last columns. The erasing signals inputted from the source signal lines 912 are inputted to the gate electrode of the second transistor 902 through the first transistor 901 connected to each of the source signal lines. At this time, current supply from the current supply line 917 to the light-emitting element 903 is stopped by the signal inputted to the second transistor 902. Then, the light-emitting element 903 is forcibly made to emit no light. For example, when the second transistor 902 is a p-channel transistor, the light-emitting element 903 emits no light by inputting a High Level signal to the gate electrode of the second transistor 902. On the other hand, when the second transistor 902 is an n-channel transistor, the light-emitting element 903 emits no light by inputting a Low Level signal to the gate electrode of the second transistor 902.

It is to be noted that, as for n-th row (n is a natural number), signals for erasing are inputted by the operation as described above in an erasing period. However, as described above, another row (referred to as m-th row (m is a natural number)) may be in a writing period while the n-th row is in an erasing period. In such a case, it is necessary to input a signal for erasing into the n-th row and input a signal for writing to the m-th row by using a source signal line in the same column. Therefore, an operation explained below is preferable.

Immediately after the light-emitting element 903 in the n-th row is made to emit no light by the operation in the erasing period as explained above, the gate signal line 911 and the erasing gate signal line driver circuit 914 are made to be disconnected to each other, and the switch 920 is switched to connect the source signal line 912 and the source signal line driver circuit 915. Then, in addition to connecting the source signal line 912 to the source signal line driver circuit 915, the gate signal line 911 is connected to the writing gate signal line driver circuit 913. Then, a signal is selectively inputted to the gate signal line 911 in the m-th row from the writing gate signal line driver circuit 913 to turn on the first transistor 901, and signals for writing are inputted to the source signal lines 912 in the first to last columns from the source signal line driver circuit 915. This signal makes the light-emitting element 903 in the m-th row be in an emission or non-emission state.

Immediately after the writing period for the m-th row is completed as described above, an erasing period for an (n+1)-th row is started. For that purpose, the gate signal line 911 and the writing gate signal line driver circuit 913 are made to be disconnected to each other, and the switch 920 is switched to connect the source signal line 912 and the power source 916. Further, the gate signal line 911 is made to be disconnected to the writing gate signal line driver circuit 913, and to be connected to the erasing gate signal line driver circuit 914. Then, a signal is selectively inputted to the gate signal line in the (n+1)-th row from the erasing gate signal line driver circuit 914 to turn on the first transistor 901, and an erasing signal is inputted from the power source 916. Immediately after the erasing period for the (n+1)-th row is thus completed, a writing period for the (m+1)-th row is started. Then, an erasing period and a writing period may be repeated in the same way until an erasing period for the last row is completed.

Although a mode in which the writing period for the m-th row is provided between the erasing period for the n-th row and the erasing period for the (n+1)-th row is explained in this embodiment mode, the present invention is not limited thereto. The writing period for the m-th row may be provided between an erasing period for a (n−1)-th row and an erasing period for the n-th row as well.

In addition, in this embodiment mode, an operation is repeated, in which the erasing gate signal line driver circuit 914 and one gate signal line are made to be disconnected to each other as well as the writing gate signal line driver circuit 913 and another gate signal line are made to be connected to each other when the non-emission period 504d is provided as in the sub-frame 504. This type of operation may also be performed in a frame in which a non-emission period is not particularly provided.

### Embodiment Mode 9

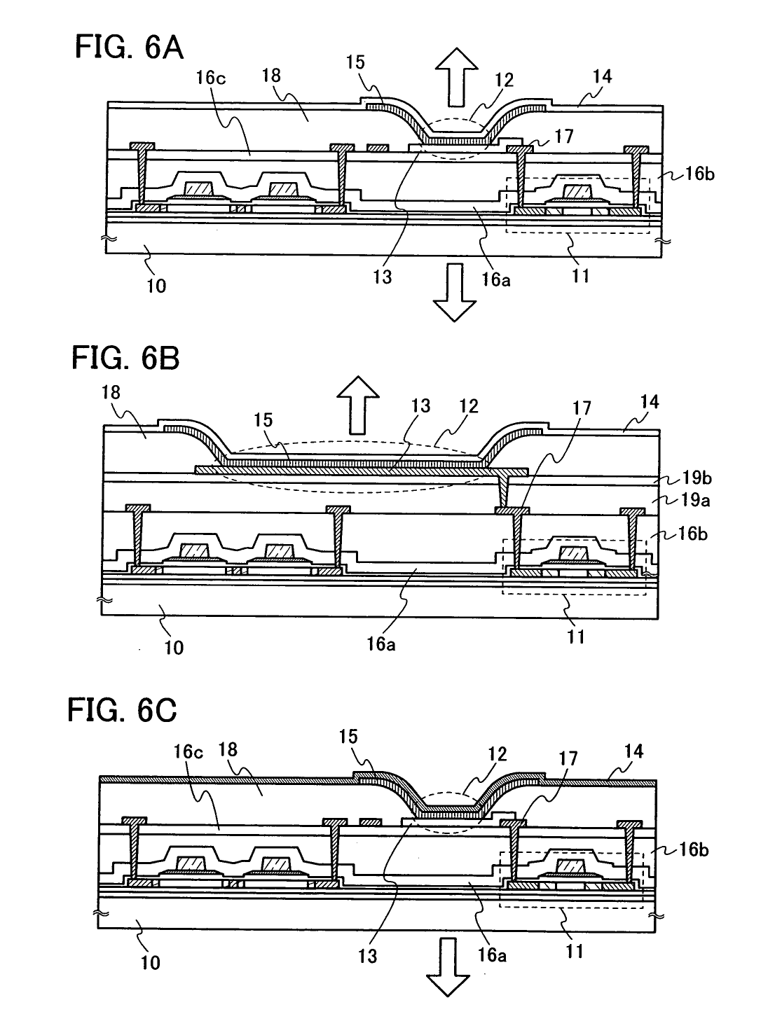

One mode of a light-emitting device including a light-emitting element of the present invention will be explained with reference to cross-sectional views of FIGS. 6A to 6C.

In each of FIGS. 6A to 6C, a box-shaped portion surrounded by a dotted line is a transistor 11 provided for driving a light-emitting element 12 of the present invention. The light-emitting element 12 includes, as explained in Embodiment Modes 5 and 6, a layer 15 between a first electrode 13 and a second electrode 14, and is a light-emitting element including a light-emitting layer and/or a hole injecting layer which are/is formed by using a light-emitting element material of the present invention which is formed by using a carbazole derivative of the present invention in the layer 15. In addition, the light-emitting element 12 may include a hole transporting layer formed by using the carbazole derivative of the present invention as explained in Embodiment Mode 7. A drain of the transistor 11 and the first electrode 13 are electrically connected to each other by a wiring 17 passing through a first interlayer insulating film 16 (16a, 16b, and 16c). In addition, the light-emitting element 12 is separated from another light-emitting element which is adjacently provided by a partition wall 18. The light-emitting device having such a structure of the present invention is provided over a substrate 10 in this embodiment mode.

Further, the transistor 11 shown in each of FIGS. 6A to 6C is a top gate type in which a gate electrode is provided on an opposite side of a substrate with a semiconductor layer as a center. However, a structure of the transistor 11 is not particularly limited, and a bottom gate type may also be employed, for example. In addition, in a case of a bottom gate type, a channel protection type in which a protection film is formed over a semiconductor layer which forms a channel, or a channel etch type in which part of a semiconductor layer which forms a channel is concave may also be employed.

In addition, a semiconductor layer included in the transistor 11 may be either crystalline or noncrystalline. Alternatively, the transistor 11 may be semiamorphous.

The following describes a semi-amorphous semiconductor. The semi-amorphous semiconductor is a semiconductor that has an intermediate structure between amorphous and crystalline (including single-crystal or polycrystalline) structures and has a third state that is stable in terms of free energy, and includes a crystalline region that has short range order and lattice distortion. Further, a crystal grain of 0.5 to 20 nm is included in at least part of a region in a film of the semi-amorphous semiconductor. Raman spectrum is shifted to a wave number side lower than 520 cm⁻¹. The diffraction peaks of (111) and (220), which are believed to be derived from silicon crystal lattice, are observed by the X-ray diffraction. The semi-amorphous semiconductor contains hydrogen or halogen of at least 1 atomic % or more for terminating dangling bonds. The semi-amorphous semiconductor is also referred to as a so-called microcrystalline semiconductor. A microcrystalline semiconductor can be formed by glow discharge decomposition of silicide gas (plasma CVD). As the silicide gas, SiH₄, Si₂H₆, SiH₂Cl₂, SiHCl₃, SiCl₄, SiF₄, or the like can be used. The silicide gas may also be diluted with H₂, or a mixture of H₂ and one or more of rare gas elements of He, Ar, Kr, and Ne. The dilution ratio is set to be in a range of 2 to 1000 times. The pressure is set to be approximately in the range of 0.1 to 133 Pa. The power frequency is set to be 1 to 120 MHz, preferably, 13 to 60 MHz. A substrate heating temperature may be set to be 300° C. or less, more preferably 100 to 250° C. As for impurity elements contained in the film, each concentration of impurities for atmospheric constituents such as oxygen, nitrogen and carbon is preferably set to be 1×10²⁰/cm³ or less. In particular, the oxygen concentration is set to be 5×10¹⁹/cm³ or less, preferably 1×10¹⁹/cm³ or less.

As a specific example of a crystalline semiconductor layer, a semiconductor layer formed of single-crystalline or polycrystalline silicon, silicon germanium, or the like can be given. The semiconductor layer may be formed by laser crystallization, or crystallization by a solid-phase growth method using nickel or the like, for example.

In a case of forming the semiconductor layer from an amorphous substance, for example amorphous silicon, it is preferable that the light-emitting device have a circuit in which the transistor 11 and other transistors (transistors constituting a circuit for driving the light-emitting element) are all n-channel transistors. Other than that, the light-emitting device may have a circuit including any one type of an n-channel transistor and a p-channel transistor, or may have a circuit including both types of the transistors.

Furthermore, the first interlayer insulating film 16 may be a multilayer as shown in FIGS. 6A to 6C, or a single layer. It is to be noted that the first interlayer insulating film 16a contains an inorganic substance such as silicon oxide or silicon nitride, and the first interlayer insulating film 16b contains acrylic or siloxane (siloxane has a skeleton structure which is structured by a bond of silicon (Si) and oxygen (O), and has a fluoro group, hydrogen, or an organic group (such as an alkyl group or aromatic hydrocarbon) as a substituent), or a substance such as silicon oxide which can be formed by coating. Furthermore, the first interlayer insulating film 16c is formed of a silicon nitride film containing argon (Ar). It is to be noted that the substances forming each layer are not particularly limited, and substances other than the substance described here may also be used. Also, a layer formed using a substance other than these substances may also be combined. In this way, the first interlayer insulating film 16 may be formed by using both an inorganic substance and an organic substance, or one of an inorganic substance and an organic substance.

As for the partition layer 18, it is preferable that an edge portion have a shape varying continuously in curvature radius. The partition wall 18 is formed by using acrylic, siloxane, resist, silicon oxide, or the like. The partition wall 18 may be formed of any one of an inorganic film or an organic film, or both of the inorganic film and the organic film.

In each of FIGS. 6A and 6C, only the first interlayer insulating film 16 is provided between the transistor 11 and the light-emitting element 12. However, as shown in FIG. 6B, a second interlayer insulating film 19 (19a and 19b) may be provided in addition to the first interlayer insulating layer 16 (16a and 16b). In the light-emitting device shown in FIG. 6B, the first electrode 13 is connected to the wiring 17 by passing through the second interlayer insulating film 19.

The second interlayer insulating film 19 may be a multilayer or a single layer, as in the first interlayer insulating film 16. The second interlayer insulating film 19a is formed by using acrylic, siloxane or a substance such as silicon oxide which can be formed by coating. Furthermore, the second interlayer insulating film 19b is formed by using a silicon nitride film containing argon (Ar). It is to be noted that the substances forming each layer are not particularly limited, and substances other than the substances described here may also be used. Also, a layer formed using a substance other than these substances may be combined. In this manner, the second interlayer insulating film 19 may be formed by using both an inorganic substance and an organic substance, or one of the inorganic substance and the organic substance.

In the light-emitting element 12, in a case where both of the first electrode and the second electrode are formed from a substance having a light-transmitting property, light emission can be extracted from both of the first electrode 13 side and the second electrode 14 side as shown by an outline arrow in FIG. 6A. In addition, in a case where only the second electrode 14 is formed using a substance having a light-transmitting property, light emission can be extracted from only the second electrode 14 side as shown by an outline arrow of FIG. 6B. In this case, it is preferable that the first electrode 13 is formed by using a material having high reflectivity or a film formed by using a material having high reflectivity (reflective film) be provided below the first electrode 13. Moreover, in a case where only the first electrode 13 is formed by using a substance having a light-transmitting property, light emission can be extracted from only the first electrode 13 side, as shown by an outline arrow of FIG. 6C. In this case, it is preferable that the second electrode 14 is formed by using a material having high reflectivity, or a reflective film be provided above the second electrode 14.

In addition, in the light-emitting element 12, the layer 15 is formed so as to operate when voltage is applied so that electric potential of the second electrode 14 is higher than that of the first electrode 13, or the layer 15 is stacked so as to operate when voltage is applied so that electric potential of the second electrode 14 is lower than that of the first electrode 13. In the former case, the transistor 11 is an n-channel transistor, and in the latter case, the transistor 11 is a p-channel transistor.

### Embodiment Mode 10

Each of the light-emitting elements of the present invention explained in Embodiment Modes 5 to 7 has resistance to repeated oxidation reactions and can emit light in a favorable state for a long time. Therefore, by using the light-emitting element of the present invention, a light-emitting device capable of providing a favorable display image or the like for a long time can be obtained. In addition, the light-emitting element of the present invention explained in Embodiment Mode 4 can exhibit blue light emission with favorable chromaticity. Therefore, by using the light-emitting element of the present invention, a light-emitting device which exhibits blue light emission with favorable chromaticity and displays an image with excellent colors can be obtained.

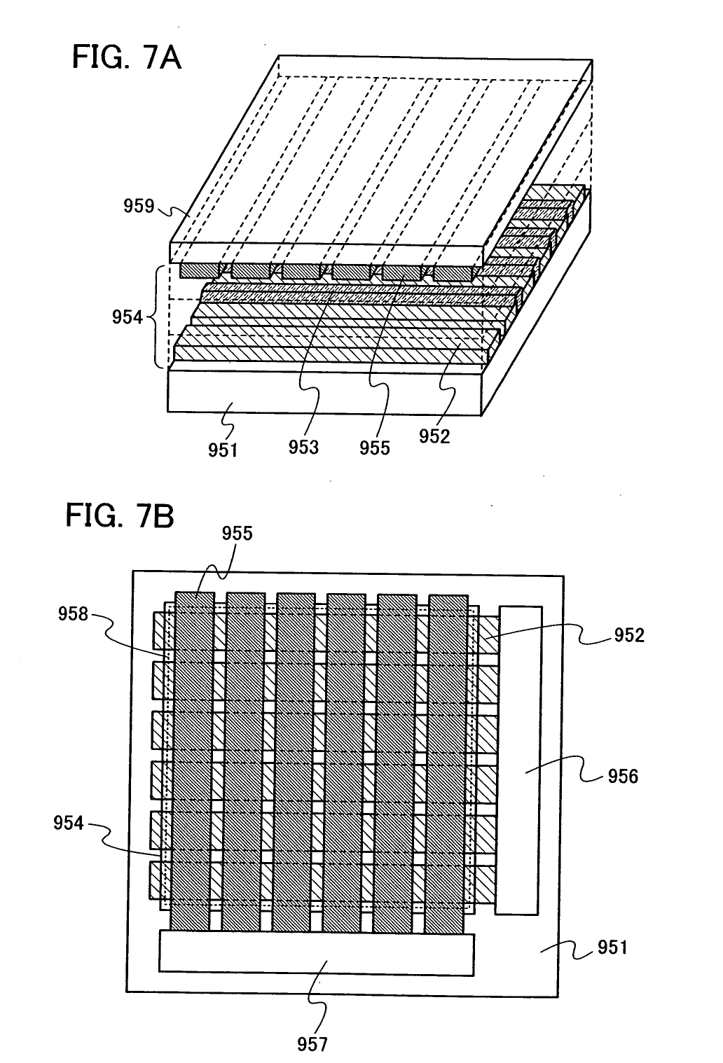

In this embodiment mode, a passive light-emitting device to which the present invention is applied will be explained with reference to FIGS. 7A and 7B. FIGS. 7A and 7B are a perspective view and a top view of a passive light-emitting device to which the present invention is applied, respectively. In particular, FIG. 7A is a perspective view of a portion surrounded by a dotted line 958 of FIG. 7B. In FIGS. 7A and 7B, the same portions are denoted by the same reference numerals. In FIG. 7A, a plurality of first electrodes 952 are provided in parallel over a substrate 951. Each of edge portions of the first electrodes 952 is covered with a partition wall layer 953. It is to be noted that, although a partition wall layer which covers the first electrode 952 on the most front side is not shown so that the positions of the first electrode 952 and the partition wall layer 953 that are provided over the first substrate 951 are easily recognized, an edge portion of the first electrode 952 on the most front side is actually covered with the partition wall. A plurality of second electrodes 955 are provided in parallel so as to intersect with the plurality of the first electrodes 952 above the first electrode 952. A layer 954 is provided between the first electrode 952 and the second electrode 955. The layer 954 includes a light-emitting layer and/or a hole injecting layer formed by using a light-emitting element material of the present invention manufactured by using a carbazole derivative of the present invention. In addition, the layer 954 may have a hole transporting layer formed by using the carbazole derivative of the present invention. It is to be noted that the layer 954 may be a single layer including only a light-emitting layer, or a multilayer including a hole transporting layer, a hole injecting layer, an electron transporting layer, an electron injecting layer, or the like, in addition to the light-emitting layer. A second substrate 959 is provided over the second electrode 955.

As shown in FIG. 7B, the first electrode 952 is connected to a first driver circuit 956, and the second electrode 955 is connected to a second driver circuit 957. A portion where the first electrode 952 and the second electrode 955 are intersected with each other forms a light-emitting element of the present invention which is formed by interposing a light-emitting layer between electrodes. The light-emitting element of the present invention selected by a signal from the first driver circuit 956 and the second driver circuit 957 emits light. Light emission is extracted to outside through the first electrode 952 and/or the second electrode 955. Then, light emissions from a plurality of light-emitting elements are combined to display an image. It is to be noted that although the partition wall layer 953 and the second substrate 959 are not shown in FIG. 7B so that the positions of the first electrode 952 and the second electrode 955 are easily recognized, the partition wall layer 953 and the second substrate 959 are actually provided as shown in FIG. 7A.

A material for forming the first electrode 952 and the second electrode 955 is not particularly limited; however, it is preferable that the first electrode 952 and the second electrode 955 be formed by using a transparent conductive material so that one of the electrodes or both of the electrodes can transmit visible light. In addition, materials for the first substrate 951 and the second substrate 959 are not particularly limited, and each of the first substrate 951 and the second substrate 959 may be formed by using a material having flexibility with a resin such as plastic, in addition to a glass substrate. A material for the partition wall layer 953 is not particularly limited either, and the partition wall layer 953 may be formed by using either an inorganic substance or an organic substance, or both of the inorganic substance and the organic substance. Besides, the partition wall layer 953 may be formed by using siloxane.

Further, the layers 954 may be formed separately for light-emitting elements each exhibiting a different emission color. For example, by providing the layers 954 for light-emitting elements each emitting red light, green light, or blue light, a light-emitting device capable of multicolor display can be obtained.

### Embodiment Mode 11

A light-emitting device having a light-emitting element manufactured by using a light-emitting element material of the present invention has resistance to repeated oxidation reactions and can emit light in a favorable state for a long time. Therefore, by using such a light-emitting device of the present invention for a display portion or a lighting portion, an electronic device capable of providing a favorable display image for a long time, or an electornic device capable of lighting favorably for a long time can be obtained.

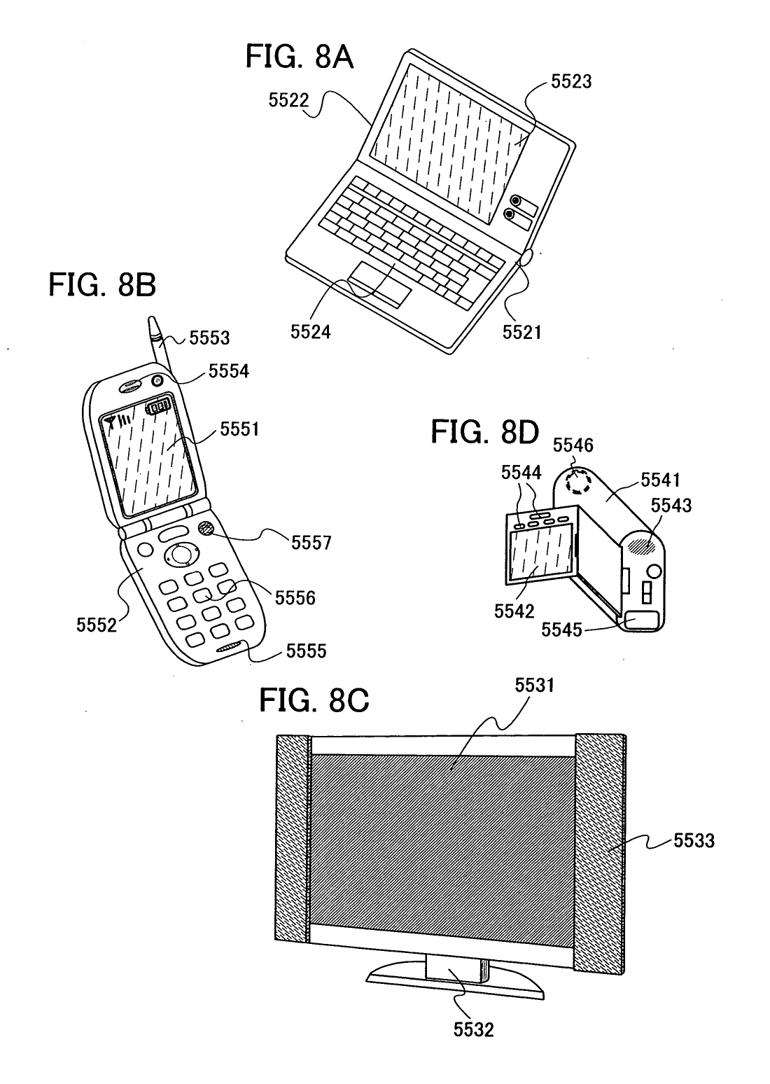

One embodiment mode of an electronic device mounted with a light-emitting device to which the present invention is applied is shown in FIGS. 8A to 8D.

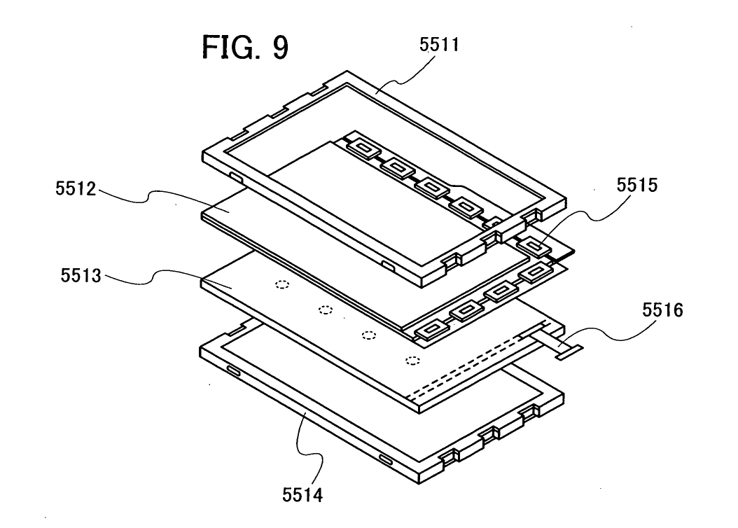

FIG. 8A shows a personal computer manufactured by applying the present invention, which includes a main body 5521, a housing 5522, a display portion 5523, a keyboard 5524, and the like. When a light-emitting device (a light-emitting device including a structure as explained in Embodiment Modes 8 to 10, for example) using the light-emitting element of the present invention as explained in Embodiment Modes 5 to 7 as a pixel is incorporated as a display portion, a personal computer, in which there are few defects in the display portion, a display image is not misidentified, and a display image with excellent colors, can be completed. In addition, even by incorporating a light-emitting device using the light-emitting element of the present invention as a light source as a backlight, a personal computer can be completed. Specifically, as shown in FIG. 9, a lighting device in which a liquid crystal display device 5512 and a light-emitting device 5513 are set in a housing 5511 and a housing 5514 may be incorporated as a display portion. It is to be noted that, as shown in FIG. 9, an external input terminal 5515 is attached to the liquid crystal display device 5512 and an external input terminal 5516 is attached to the light-emitting device 5513.

FIG. 8B shows a telephone set manufactured by applying the present invention, which includes a main body 5552, a display portion 5551, an audio output portion 5554, an audio input portion 5555, operation switches 5556 and 5557, an antenna 5553, and the like. By incorporating a light-emitting device having the light-emitting element of the present invention as a display portion, a telephone set, in which there are few defects in the display portion, a display image is not misidentified, and a display image with excellent colors, can be completed.

FIG. 8C shows a television set manufactured by applying the present invention, which includes a display portion 5531, a housing 5532, a speaker 5533, and the like. By incorporating a light-emitting device having the light-emitting element of the present invention as a display portion, a television set, in which there are few defects in the display portion, a display image is not misidentified, and a display image with excellent colors, can be completed.

FIG. 8D shows a recording/reproducing device, which includes a main body 5541, a display portion 5542, an audio input portion 5543, an operation switch portion 5544, a battery portion 5545, and an image receiving portion 5546, and the like. By incorporating a light-emitting device having the light-emitting element of the present invention as a display portion, a recording/reproducing device in which there are few defects in the display portion, a display image is not misidentified, and a display image with excellent colors, can be completed.

The above-described light-emitting device of the present invention is highly suitable for being used as a display portion of various electronic devices. It is to be noted that an electronic device is not limited to the electronic devices described in this embodiment mode, and the electronic device may be other electronic devices such as a navigation device.

### Embodiment 1

As one embodiment of the present invention, a synthesis of a carbazole derivative of the present invention will be explained hereinafter.

### Synthesis Example 1

A synthesis of 3-(N-phenylamino)-9-phenylcarbazole (abbreviation: PCA) represented by a structural formula (1) will be explained.

First, 24.3 g (100 mmol) of N-phenylcarbazole was dissolved in 600 mL of glacial acetic acid, and 17.8 g (100 mmol) of N-bromosuccinimide was slowly added thereto. The mixture was stirred at a room temperature for about 20 hours. This glacial acetic acid solution was dropped into 1 L of ice water while stirring it. A precipitated white solid was washed with water three times. This solid was dissolved in 150 mL of diethyl ether, and washed with a saturated sodium hydrogen carbonate solution and water. This organic layer was dried with magnesium sulfate, and filtered. The obtained filtrate was condensed. About 50 mL of methanol is added to the obtained residue, and uniformly dissolved therein by emitting ultrasonic wave. This solution was left still to precipitate a white solid. This solid was filtered and the residue was dried to obtain 28.4 g (yield: 88%) of 3-bromo-9-phenylcarbazole, which was white powder.

A synthesis scheme (f-1) of 3-bromo-9-phenylcarbazole is shown next.

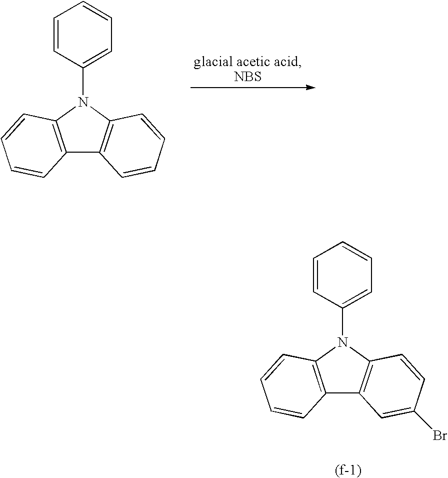

Next, under nitrogen, 110 mL of dehydrated xylene and 7.0 g (75 mmol) of aniline were added to a mixture of 19 g (60 mmol) of 3-bromo-9-phenylcarbazole, 340 mg (0.6 mmol) of bis(dibenzylideneacetone)palladium(0) (abbreviation: Pd(dba)₂), 1.6 g (3.0 mmol) of 1,1-bis(diphenylphosphino)ferrocene (abbreviation: DPPF), and 13 g (180 mmol) of sodium-tert-butoxide (abbreviation: tert-BuONa). This was stirred while heating at 90° C. under a nitrogen atmosphere for 7.5 hours. After the termination of the reaction, about 500 mL of hot toluene was added to the suspension and this suspension was filtered through Florisil®, alumina, and Celite. The obtained filtrate was condensed, its residue was mixed with hexane-ethyl acetate, and ultrasonic wave was emitted. The obtained suspension was filtered and the residue was dried to obtain 15 g (yield: 75%) of cream-colored powder. This cream-colored powder was identified as 3-(N-phenylamino)-9-phenylcarbazole (abbreviation: PCA) by a nuclear magnetic resonance method (¹H-NMR).

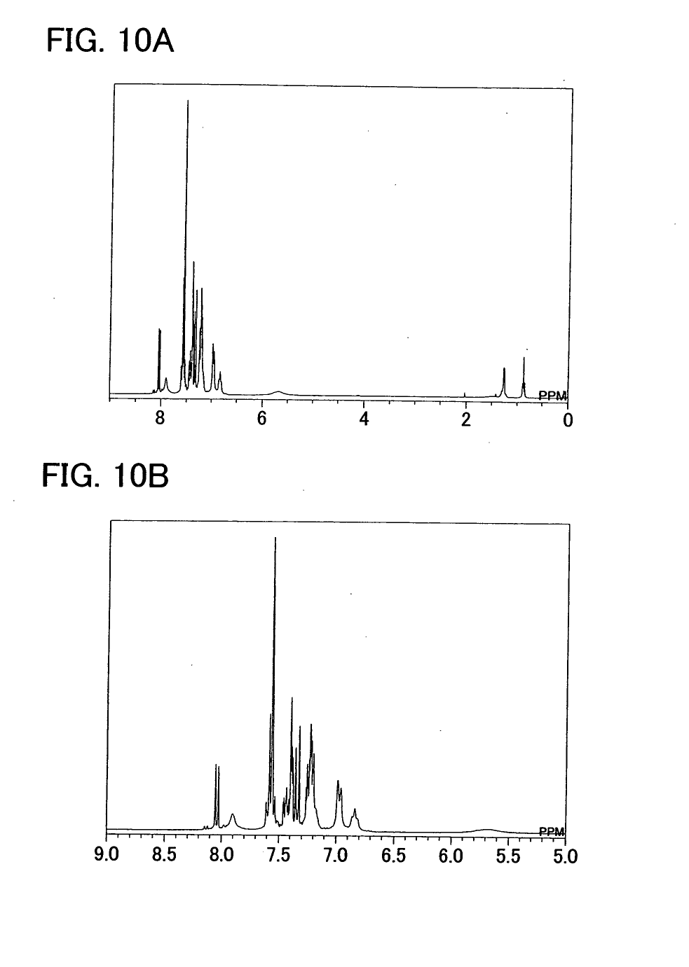

¹H-NMR of this compound is shown next. ¹H-NMR charts are shown in FIGS. 10A and 10B. It is to be noted that FIG. 10B is an enlarged chart of a portion of 5.0 to 9.0 ppm in FIG. 10A.

¹H-NMR (300 MHz, CDCl₃); δ=6.84 (t, J=6.9 Hz, 1H), 6.97 (d, J=7.8 Hz, 2H), 7.20-7.61 (m, 13H), 7.90 (s, 1H), 8.04 (d, J=7.8 Hz, 1H)

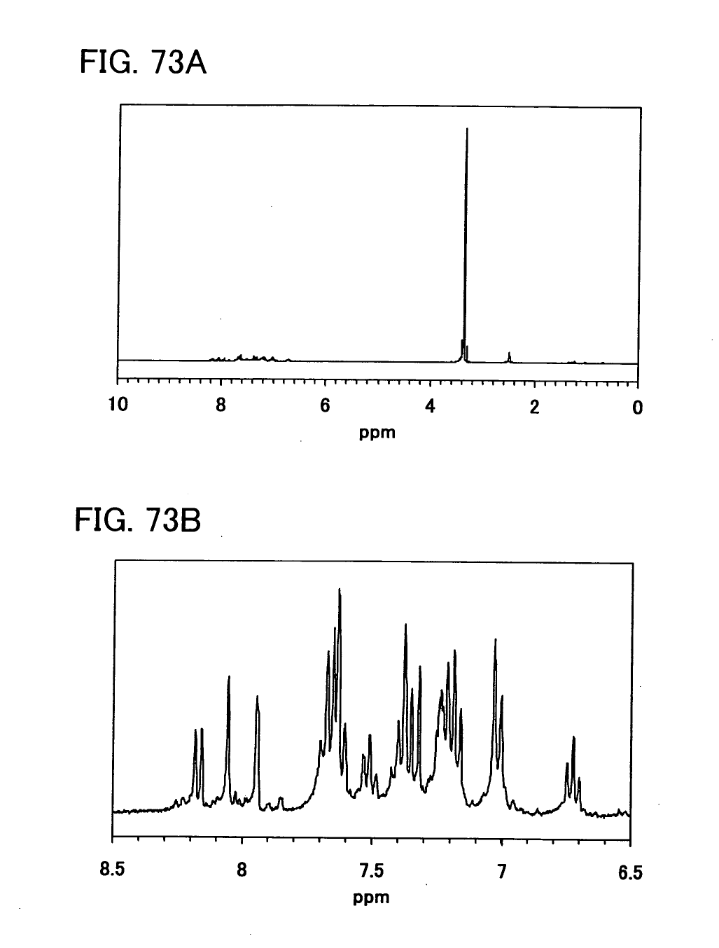

¹H-NMR data in a case of using DMSO (DiMethyl SulfOxide) for a heavy solvent is shown below, and ¹H-NMR charts are shown in FIGS. 73A and 73B. It is to be noted that FIG. 73B is an enlarged chart of a portion of 6.5 to 8.5 ppm in FIG. 73A.

¹H-NMR (300 MHz, DMSO-d₆); δ=6.73 (t, J=7.5 Hz, 1H), 7.02 (d, J=8.1 Hz, 2H), 7.16-7.70 (m, 12H), 7.95 (s, 1H), 8.06 (s, 1H), 8.17 (d, J=7.8 Hz, 1H)

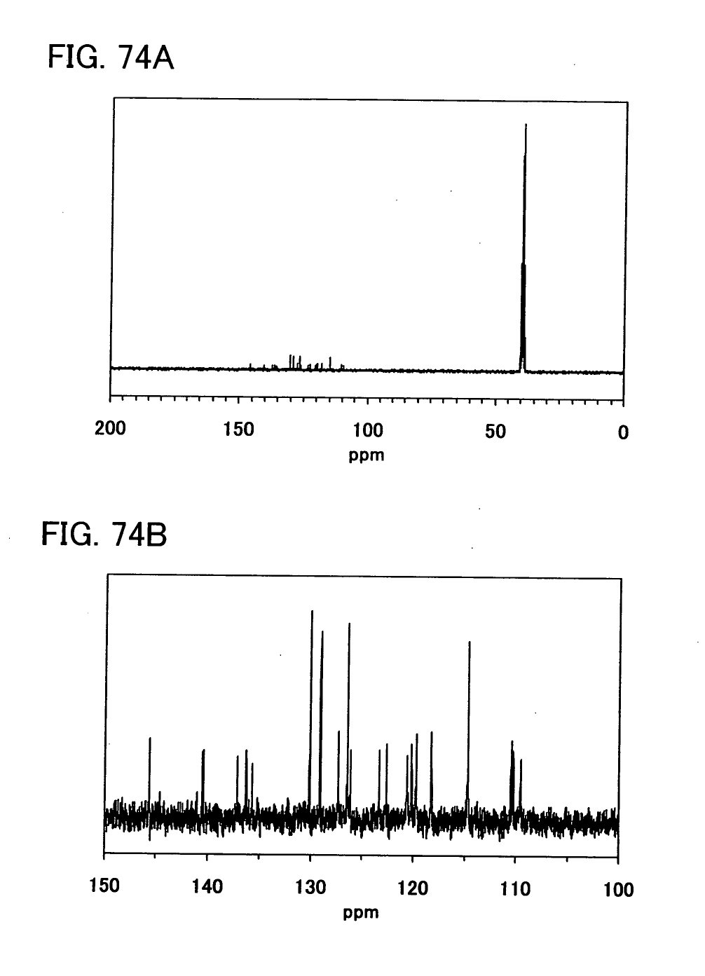

Next, ¹³C-NMR data in a case of using DMSO for a heavy solvent is shown below, and ¹³C-NMR charts are shown in FIGS. 74A and 74B. It is to be noted that FIG. 74B is an enlarged chart of a portion of 100 to 150 ppm in FIG. 74A.

¹³C-NMR (75.5 MHz, DMSO-d₆); δ=109.55, 110.30, 110.49, 114.71, 118.22, 119.70, 120.14, 120.61, 122.58, 123.35, 126.18, 126.48, 127.37, 129.15, 130.14, 135.71, 136.27, 137.11, 140.41, 145.61

A synthesis scheme (f-2) of 3-(N-phenylamino)-9-phenylcarbazole is shown next.

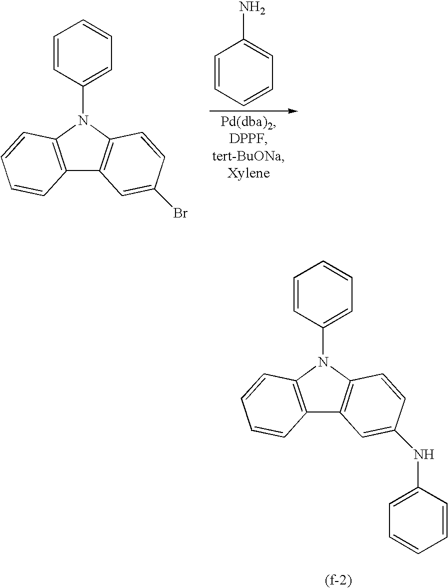

### Synthesis Example 2

A synthesis method of 3-[N-(1-naphthyl)amino]-9-phenylcarbazole (abbreviation: PCN) represented by a structural formula (2) will be explained.

Under nitrogen, 12 mL of dehydrated xylene was added to a mixture of 3.7 g (10 mmol) of 3-iodo-9-phenylcarbazole, 1.6 g (5 mmol) of 1-aminonaphthalene, 60 mg (0.1 mmol) of bis(dibenzylideneacetone)palladium(0), 200 μL (0.5 mmol) of a hexane solution containing 49 wt % of tri-tert-butylphosphine, and 3 g (30 mmol) of sodium-tert-butoxide. This was stirred while heating at a temperature of 90° C. under a nitrogen atmosphere for 7 hours. After the termination of the reaction, about 200 mL of hot toluene was added to the suspension and this was filtered through Florisil®, alumina, and Celite. The obtained filtrate was condensed and its residue was separated by using silica gel column chromatography (toluene:hexane=1:1). The obtained solid was recrystallized with ethyl acetate-hexane, and 3-[N-(1-naphthyl)amino]-9-phenylcarbazole (abbreviation: PCN), which was cream-colored powder, was obtained (1.5 g, yield: 79%).

NMR data is shown below.

¹H-NMR (300 MHz, DMSO-d₆); δ=7.13-7.71 (m, 15H), 7.85-7.88 (m, 1H), 8.03 (s, 1H), 8.15 (d, J=7.8 Hz, 1H), 8.24 (s, 1H), 8.36-8.39 (m, 1H)

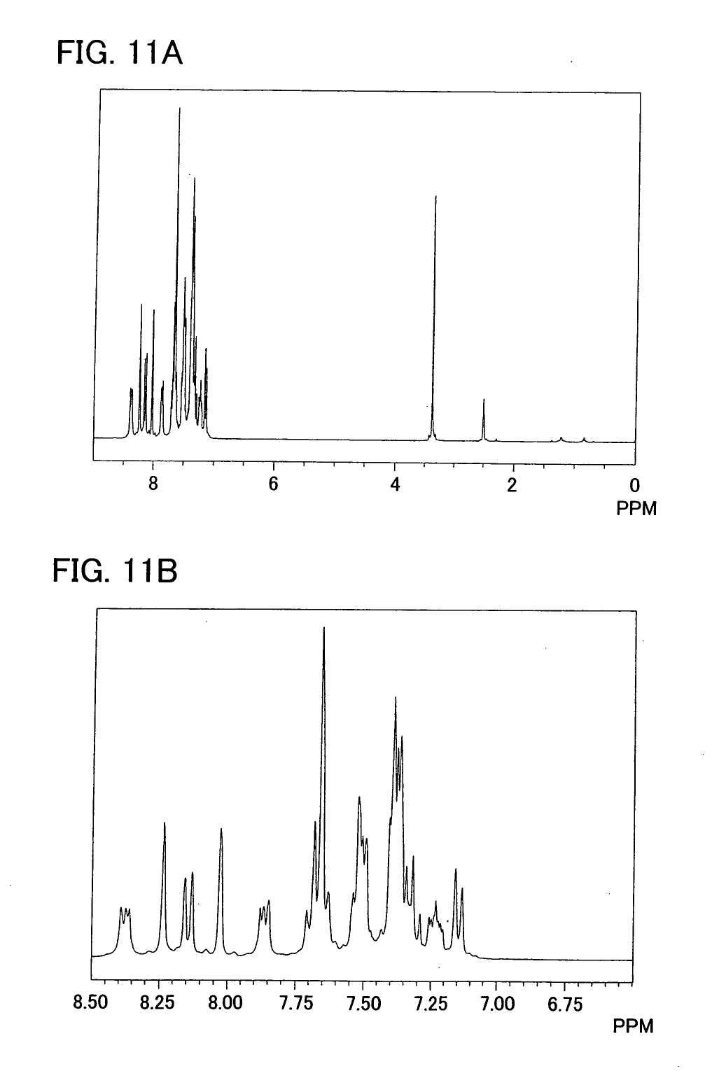

A ¹H-NMR chart is shown in FIG. 11A, and an enlarged chart of a portion of 6.50 to 8.50 ppm in FIG. 11A is shown in FIG. 11B.

A synthesis scheme (g-1) of 3-[N-(1-naphthyl)amino]-9-phenylcarbazole is shown next.

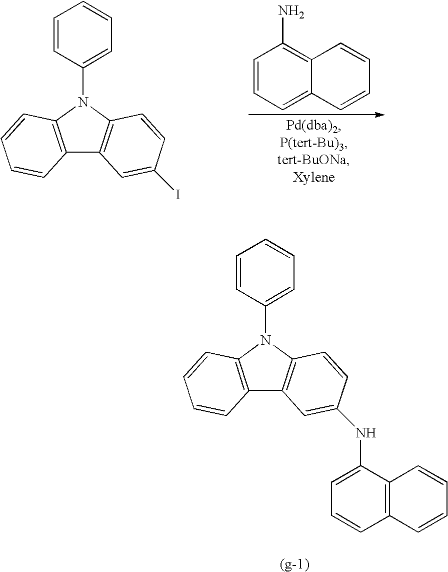

### Embodiment 2

### Synthesis Example 3

As one embodiment of the present invention using PCA synthesized in Synthesis Example 1, 9,10-bis{4-[N-(9-phenylcarbazol-3-yl)-N-phenylamino]phenyl}-2-tert-butylanthracene (abbreviation: PCABPA) will be explained.

[Step 1]

A synthesis method of 9,10-bis(4-bromophenyl)-2-tert-butylanthracene will be explained.

Under nitrogen gas stream, 1.58 mol/L of butyl lithium hexane solution (13.4 mL) was dropped into 5.0 g of dehydrated ether solution (200 mL) of 1,4-dibromobenzene at a temperature of −78° C. After the termination of the drop, the solution was stirred at the same temperature for one hour. Dehydrated ether solution (40 mL) of 2-tert-butylanthraquinone (2.80 g) was dropped to the solution at a temperature of −78° C. Thereafter, the temperature of the reaction solution was slowly increased to a room temperature. After the solution was stirred at a room temperature for about 18 hours, water was added, and extraction was performed with ethyl acetate. An organic layer was washed with saturated saline, dried with magnesium sulfate, filtered, condensed, and the residue was purified by using silica gel chromatography (developing solvent, hexane-ethyl acetate) to obtain 5.5 g of a compound.

The obtained compound was identified as 9,10-bis(4-bromophenyl)2-tert-butyl-9,10-dihydroxy-9,10-dihydroanthracene by measurement by a nuclear magnetic resonance method (¹H-NMR).

¹H-NMR of this compound is shown below.

¹H-NMR (300 MHz, CDCl₃); δ=1.31 (s, 9H), 2.81 (s, 1H), 2.86 (s, 1H), 6.82-6.86 (m, 4H), 7.13-7.16 (m, 4H), 7.36-7.43 (m, 3H), 7.53-7.70 (m, 4H)

A synthesis scheme (h-1) of 9,10-bis(4-bromophenyl)-2-tert-butyl-9,10-dihydroxy-9,10-dihydroanthracene is shown below.

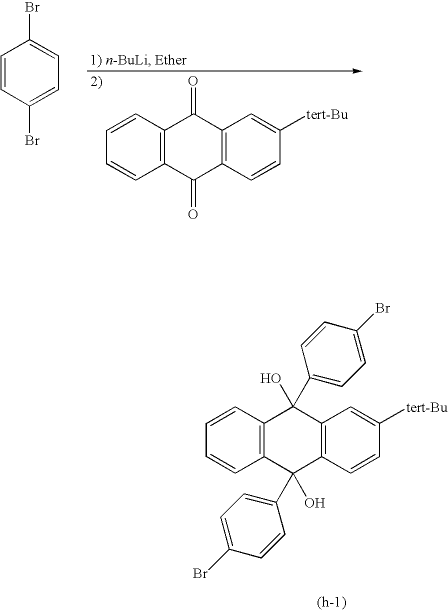

Under atmospheric air, 987 mg (1.55 mmol) of 9,10-bis(4-bromophenyl)2-tert-butyl-9,10-dihydroxy-9,10-dihydroanthracene which was synthesized as described above, 664 mg (4 mmol) of potassium iodide, and 1.48 g (14 mmol) of sodium phosphate monohydrate were suspended with 12 mL of glacial acetic acid. The mixture was heated to reflux and stirred for two hours. The reaction mixture was cooled down to a room temperature and a generated precipitate was filtered, and then the obtained solid was washed with about 50 mL of methanol. The obtained solid was dried to obtain 700 mg of a compound, which was cream-colored powder. The yield was 82%. This compound was identified as 9,10-bis(4-bromophenyl)-2-tert-butylanthracene by a measurement by a nuclear magnetic resonance method (¹H-NMR, ¹³C-NMR).

¹H-NMR and the ¹³C-NMR of this compound are shown below.

¹H-NMR (300 MHz, CDCl₃); δ=1.28 (s, 9H), 7.25-7.37 (m, 6H), 7.44-7.48 (m, 1H), 7.56-7.65 (m, 4H), 7.71-7.76 (m, 4H).

¹³C-NMR (74 MHz, CDCl₃); δ=30.8, 35.0, 120.8, 121.7, 121.7, 124.9, 125.0, 125.2, 126.4, 126.6, 126.6, 128.3, 129.4, 129.7, 129.9, 131.6, 131.6, 133.0, 133.0, 135.5, 135.7, 138.0, 138.1, 147.8

A synthesis scheme (h-2) of 9,10-bis(4-bromophenyl)-2-tert-butylanthracene is shown below. 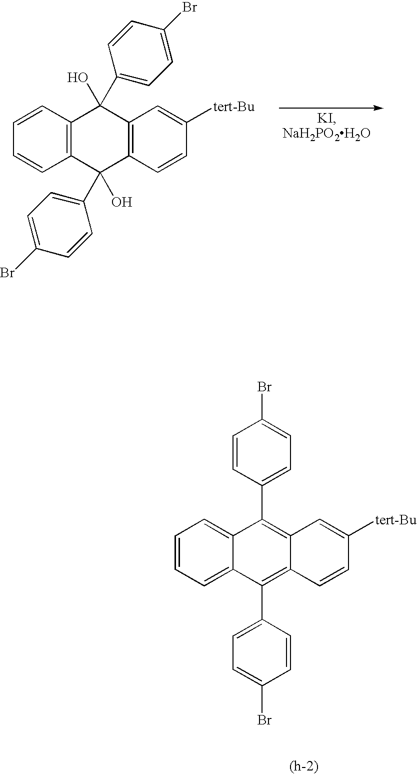
[Step 2]

A synthesis method of 9,10-bis{4-[N-(9-phenylcarbazol-3-yl)-N-phenylamino]phenyl}-2-tert-butylanthracene (abbreviation: PCABPA) will be explained.

Under nitrogen, 10 mL of dehydrated toluene was added to a mixture of 540 mg (1.0 mmol) of 9,10-bis(4-bromophenyl)-2-tert-butylanthracene, 670 mg (2.0 mmol) of 3-(N-phenylamino)-9-phenylcarbazole, 12 mg (0.02 mmol) of bis(dibenzylideneacetone)palladium(0), 110 mg (0.2 mmol) of 1,1-bis(diphenylphosphino)ferrocene, and 600 mg (6.2 mmol) of sodium-tert-butoxide. This was stirred while heating at a temperature of 90° C. under a nitrogen atmosphere for 5 hours. After the termination of the reaction, about 100 mL of toluene was added to the suspension, and the suspension was filtered through Florisil®, alumina, and Celite. The obtained filtrate was condensed to be separated by using silica gel column chromatography (toluene:hexane=1:1). This was condensed and the obtained residue was recrystallized with dichloromethane-hexane. Thus, 500 mg of yellow powder was obtained (yield: 48%). This yellow-green powder was identified as 9,10-bis{4-[N-(9-phenylcarbazol-3-yl)-N-phenylamino]phenyl}-2-tert-butylanthracene (abbreviation: PCABPA) by a nuclear magnetic resonance method (¹H-NMR).

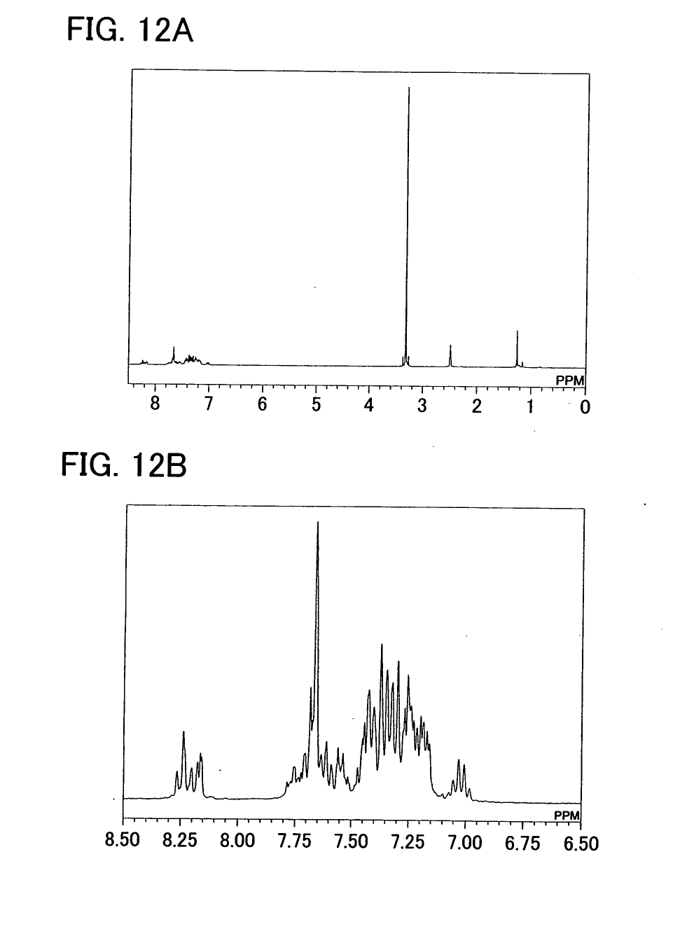

¹H-NMR of this compound is shown next. ¹H-NMR charts are shown in FIGS. 12A and 12B. It is to be noted that FIG. 12B is an enlarged chart of a portion of 6.5 to 8.5 ppm in FIG. 12A.

¹H-NMR (300 MHz, DMSO-d₆); δ=3.33 (s, 9H), 6.98-7.79 (m, 44H), 8.16-8.27 (m, 4H).

A synthesis scheme (i-1) of PCABPA is shown next.

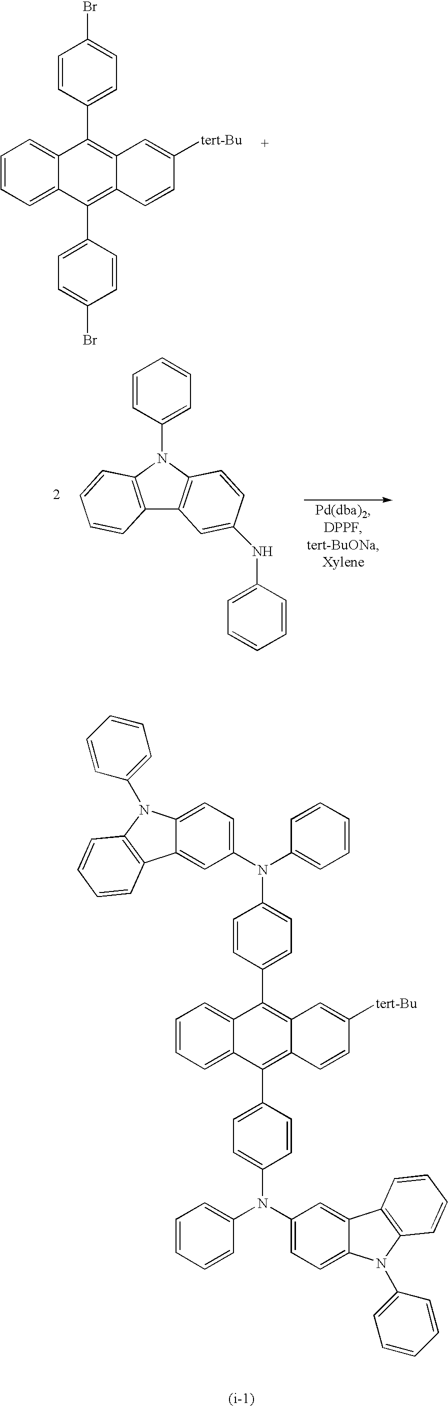

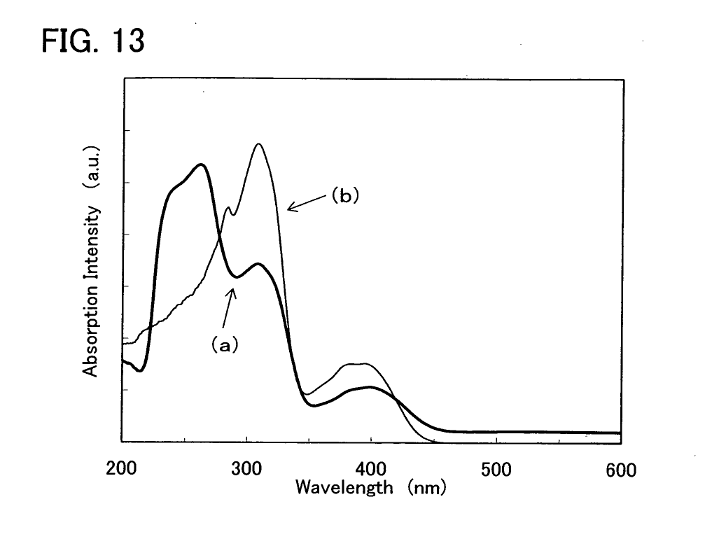

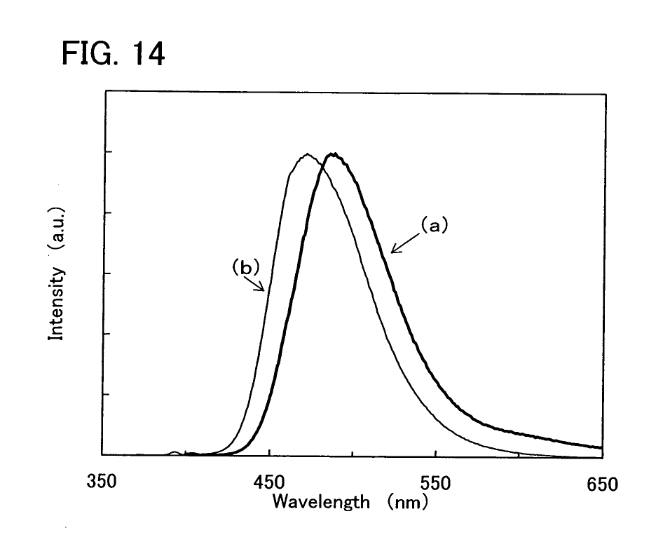

Absorption spectra of PCABPA are shown in FIG. 13. An ultraviolet-visible spectrophotometer (V-550, manufactured by JASCO Corporation) was used for a measurement. In FIG. 13, the horizontal axis indicates a wavelength (mn) and the vertical axis indicates absorption intensity (arbitrary unit). Also, (a) indicates an absorption spectrum in a state of a single film and (b) indicates an absorption spectrum in a state of being dissolved in a toluene solution. In addition, light emission spectra of PCABPA are shown in FIG. 14. In FIG. 14, the horizontal axis indicates a wavelength (nm) and the vertical axis indicates light emission intensity (arbitrary unit). Also, (a) indicates a light emission spectrum in a state of a single film (an excited wavelength: 352 nm) and (b) indicates a light emission spectrum in a state of being dissolved in a toluene solution (an excited wavelength: 390 nm). According to FIG. 14, it was found that light emission from PCABPA had a peak at 488 nm in the state of a single film, and had a peak at 472 nm in the toluene solution. These light emissions were visibly identified as blue emission color.

The obtained PCABPA was deposited by an evaporation method, and ionization potential of the compound in a state of a thin film was 5.31 eV when being measured by using a photoelectron spectrometer (AC-2, manufactured by Riken Keiki Co., Ltd.). According to this result, it was found that a HOMO level was −5.31 eV. Also, a LUMO level was −2.54 eV when the LUMO level was obtained by setting a wavelength of an absorption edge at a longer wavelength side of the absorption spectrum of the compound in a state of a thin film ((a) in FIG. 13) as an energy gap (2.77 eV).

Furthermore, when decomposition temperature T_{d} of the obtained PCABPA was measured by a thermo-gravimetric/differential thermal analyzer (TG/DTA 320, manufactured by Seiko Instruments Inc.), it was found that T_{d} was 485° C., and PCABPA showed favorable heat resistance. It is to be noted that T_{d} refers to a temperature at which the gravity becomes 95% or less with respect to the gravity at the start of the measurement under normal pressure.

Oxidation and reduction reaction characteristics of PCABPA were measured by a cyclic voltammetry (CV) measurement. An electrochemical analyzer (ALS model 600A, manufactured by BAS Inc.) was used for the measurement.

As for a solution used in the CV measurement, dehydrated dimethylformamide (DMF) was used as a solvent. Tetra-n-butylammonium perchlorate (n-Bu₄NClO₄), which is a supporting electrolyte, was dissolved so that the concentration of the tetra-n-butylammonium perchlorate was 100 mmol/L. Furthermore, PCABPA, which is an object to be measured, was dissolved therein and prepared so that the concentration thereof was 1 mmol/L. Also, a platinum electrode (PTE platinum electrode, manufactured by BAS Inc.) was used as a work electrode. A platinum electrode (VC-3 Pt counter electrode (5 cm), manufactured by BAS Inc.) was used as an auxiliary electrode. An Ag/Ag⁺ electrode (RE 5 nonaqueous reference electrode, manufactured by BAS Inc.) was used as a reference electrode.

An oxidation characteristic was measured as follows: potential of the work electrode with respect to the reference electrode was changed from −0.01 to 0.6 V. Thereafter, a scan for changing from 0.6 to −0.01 V was set to be as one cycle, and 100 cycles were measured. It is to be noted that scan speed of the CV measurement was set to be 0.1 V/s.

A reduction reaction characteristic was examined as follows: Potential of the work electrode with respect to the reference electrode was changed from −0.9 to −2.7 V. Thereafter, a scan from −2.7 to −0.9 V was set to be as one cycle, and 100 cycles were measured. It is to be noted that scan speed of the CV measurement was set to be 0.1 V/s.

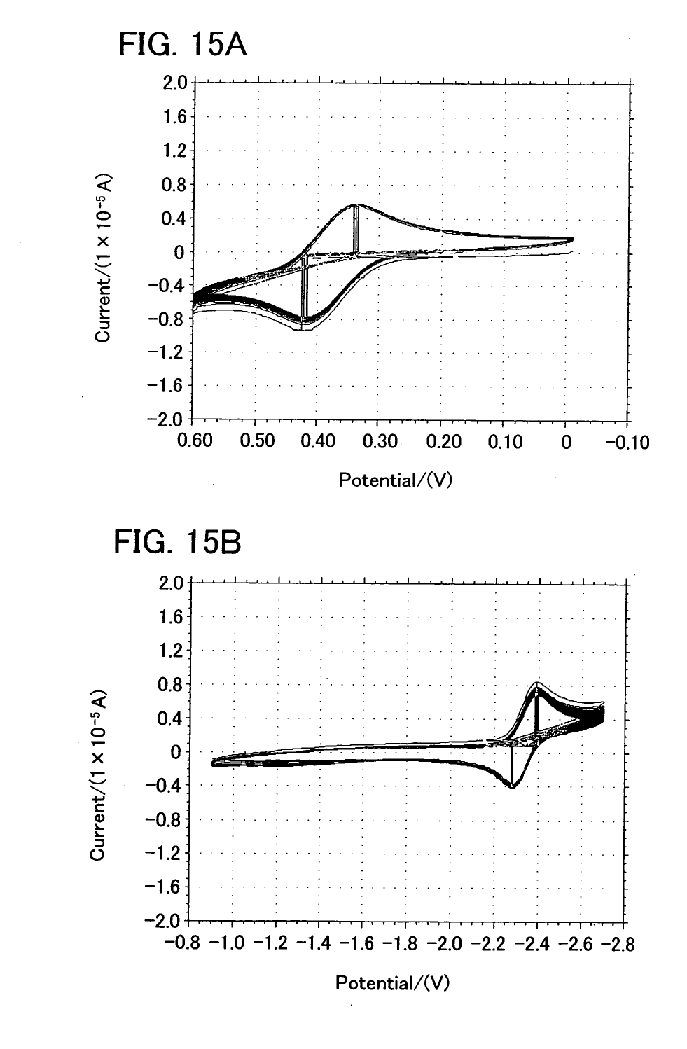

The examination result of the oxidation reaction characteristic of PCABPA is shown in FIG. 15A. Also, the examination result of the reduction reaction characteristic of PCABPA is shown in FIG. 15B. In FIGS. 15A and 15B, the horizontal axis indicates potential (V) of the work electrode with respect to the reference electrode, and the vertical axis indicates a current value (1×10⁻⁵ A) flowing between the work electrode and the auxiliary electrode.

According to FIG. 15A, it was found that potential when current which indicates oxidation in the cyclic voltammetry becomes maximum (hereinafter, referred to as oxidation peak potential) was 0.42 V (vs. Ag/Ag⁺ electrode). In addition, according to FIG. 15B, it was found that potential when current which indicates reduction in the cyclic voltammetry becomes maximum (hereinafter, referred to as reduction peak potential) was −2.39 V (vs. Ag/Ag⁺ electrode). In spite of 100 repeated cycles, changes of a peak position or peak intensity of the CV curve are hardly seen as for both the oxidation reaction and the reduction reaction. Accordingly, it was found that an anthracene derivative of the present invention is extremely stable to the oxidation and reduction reactions.

### Synthesis Example 4

As one embodiment of the present invention using PCA which was synthesized in Synthesis Example 1, a synthesis of 3-[N-(9-phenylcarbazol-3-yl)-N-phenylamino]-9-phenylcarbazole (abbreviation: PCzPCA1) will be explained.

[Step 1]

A synthesis method of 3-iodo-9-phenylcarbazole will be explained. A synthesis scheme of 3-iodo-9-phenylcarbazole is shown in (j-1).

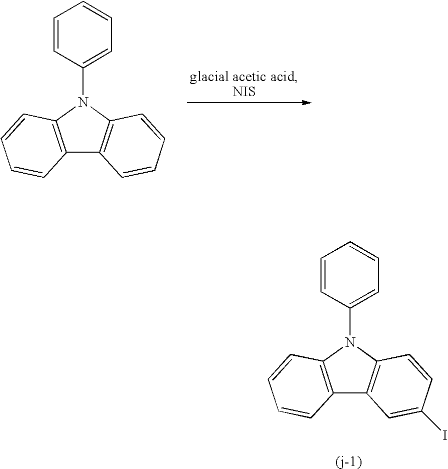

24.3 g (100 mmol) of 9-phenylcarbazole was dissolved in 600 mL of glacial acetic acid, 22.5g (100 mmol) of N-iodosuccinimide was slowly added thereto, and the mixture was stirred at a room temperature for about 20 hours. The generated precipitate was filtered, and the residue was washed with a saturated sodium hydrogen carbonate solution, water, and methanol, and then dried. 24.7 g (yield: 67%) of 3-iodo-9-phenylcarbazole, which was white powder, was obtained.

It is to be noted that 3-iodo-9-phenylcarbazole can be synthesized by the following method. 10 g (10.0 mmol) of N-phenylcarbazole, 838 mg (5.0 mmol) of potassium iodide, 1.1 g (5.0 mmol) of potassium iodate, and 30 mL of glacial acetic acid were put in a three-neck flask and refluxed at 120° C. for 1 hour. After the reaction, the reaction solution was cooled sufficiently, added to water to perform extraction with toluene. An organic layer was washed with a saturated saline once and dried with magnesium sulfate. The solution was filtered naturally, and the obtained filtrate was condensed and recrystallized with acetone and methanol. Then, 8.0 g (yield: 50%) of a white solid, which was an objective substance, was obtained. A synthesis scheme is shown in (j-2). 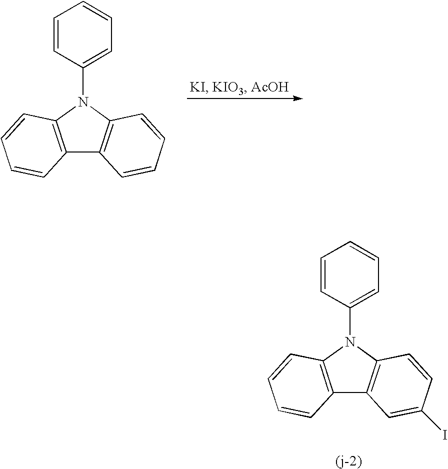
[Step 2]

A synthesis method of 3-[N-(9-phenylcarbazol-3-yl)-N-phenylamino]-9-phenylcarbazole (abbreviation: PCzPCA1) will be explained. A synthesis scheme of PCzPCA1 is shown in (j-3).

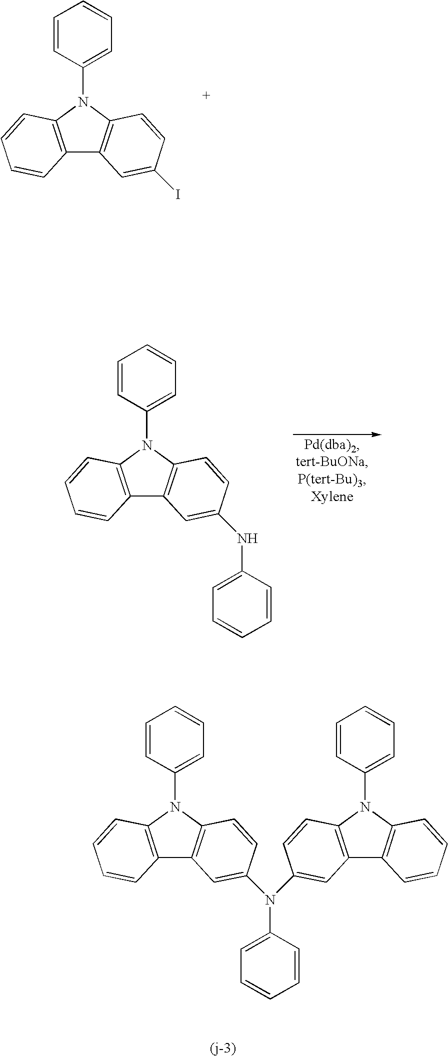

Under nitrogen, 40 mL of dehydrated xylene was added to a mixture of 3.7 g (10 mmol) of 3-iodo-9-phenylcarbazole, 3.4 g (10 mmol) of PCA, 57 mg (0.1 mmol) of bis(dibenzylideneacetone)palladium(0), 200 μL (0.5 mmol) of a hexane solution containing 49 wt % of tri-tert-butylphosphine, and 3.0 g (30 mmol) of sodium-tert-butoxide. This mixture was stirred while heating at 90° C. under a nitrogen atmosphere for 6.5 hours. After the termination of the reaction, about 500 mL of hot toluene was added to the suspension and this suspension was filtered through Florisil®, alumina, and Celite. The obtained filtrate was condensed and its residue was separated by using silica gel column chromatography (toluene:hexane=1:1). This was condensed and ethyl acetate-hexane was added to the obtained residue to conduct recrystallization. 3.2 g (yield: 56%) of 3-[N-(9-phenylcarbazol-3-yl)-N-phenylamino]-9-phenylcarbazole, which was cream-colored powder, was obtained.

NMR data is shown below.

¹H-NMR (300 MHz, DMSO-d₆); δ=6.85 (t, J=7.5 Hz, 3H), 6.92 (d, J=7.8 Hz, 2H), 7.17-7.70 (m, 22H), 8.05 (d, J=2.1 Hz, 2H), 8.12 (d, J=7.8 Hz, 2H)

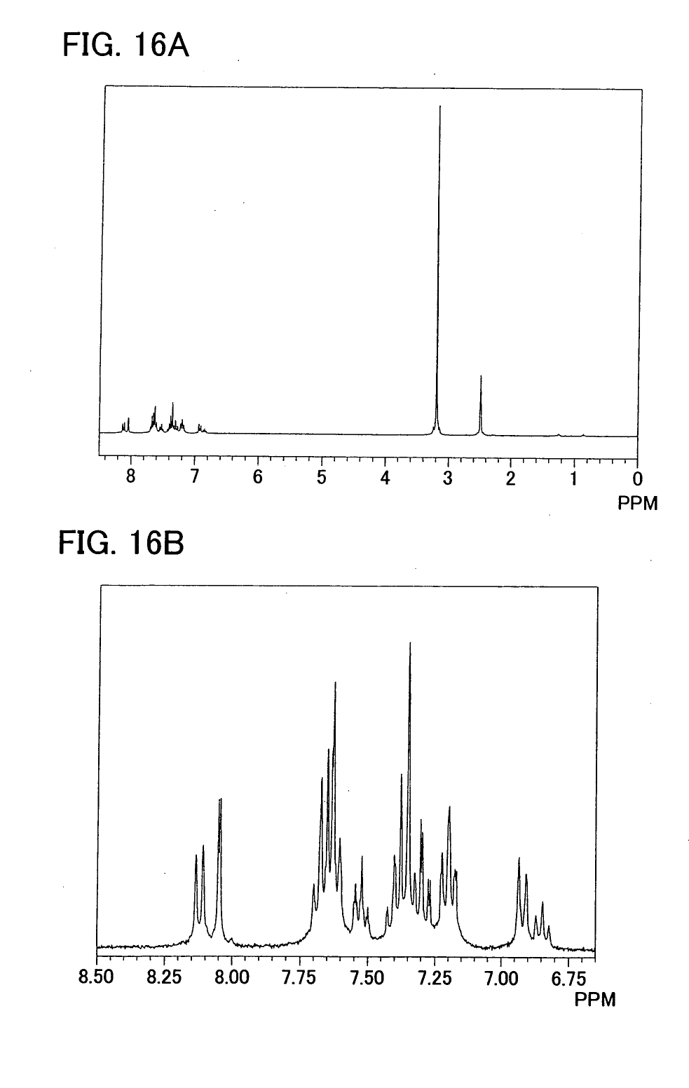

A ¹H-NMR chart is shown in FIG. 16A, and FIG. 16B is an enlarged chart of a portion of 6.50 to 8.50 ppm in FIG. 16A.

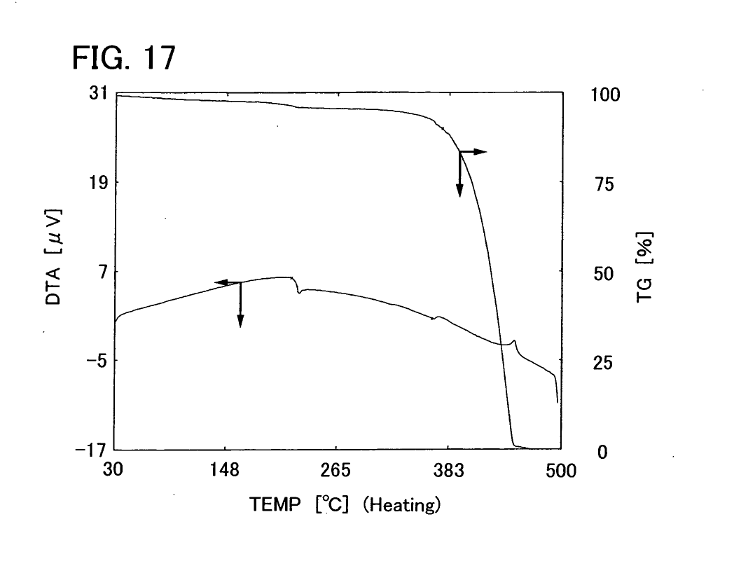

A thermogravimetry-differential thermal analysis (TG-DTA) of the obtained PCzPCA1 was performed. The result is shown in FIG. 17. In FIG. 17, the vertical axis on the left side indicates heat quantity (μV), and the vertical axis on the right side indicates gravity (%; gravity expressed assuming that gravity at the start of measurement is 100%). Furthermore, the lower horizontal axis indicates a temperature (° C.). A thermo-gravimetric/differential thermal analyzer (TG/DTA 320, manufactured by Seiko Instruments Inc.) was used for a measurement, and thermophysical properties were evaluated at a temperature rising rate of 10° C./min under a nitrogen atmosphere. As a result, from the relationship between the gravity and the temperature (thermogravimetry), the temperature T_{d}, at which the gravity becomes 95% or less with respect to the gravity at the start of the measurement, was 375° C. under normal pressure.

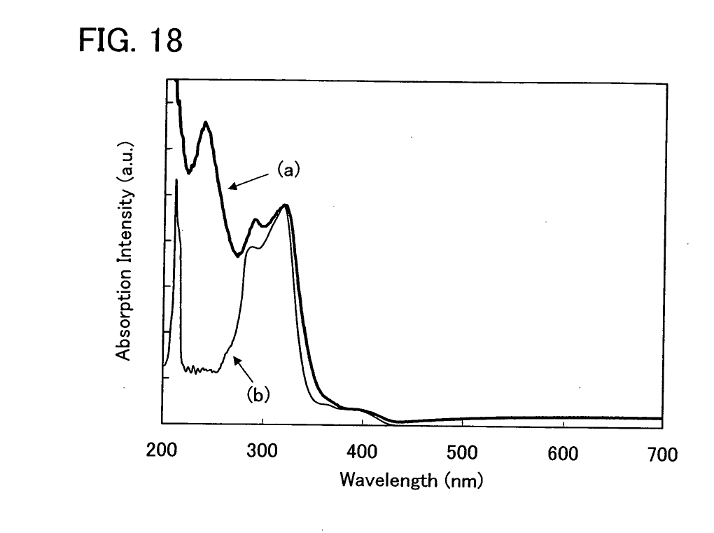

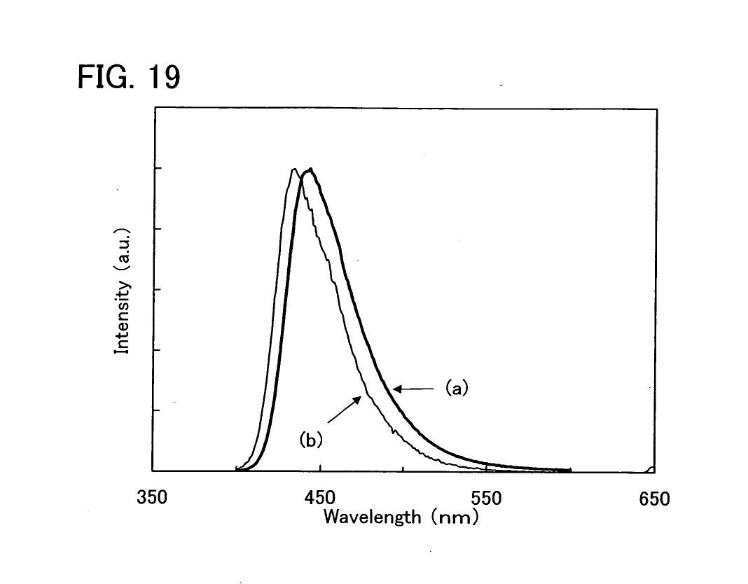

Absorption spectra of a toluene solution of PCzPCA1 and a thin film of PCzPCA1 are shown in FIG. 18. An ultraviolet-visible spectrophotometer (V-550, manufactured by JASCO Corporation) was used for a measurement. Also, (a) indicates an absorption spectrum in a state of a single film and (b) indicates an absorption spectrum in a state of being dissolved in a toluene solution. In FIG. 18, the horizontal axis indicates a wavelength (nm) and the vertical axis indicates absorption intensity (arbitrary unit). Light emission spectra of PCzPCA1 in the toluene solution and PCzPCA1 in a state of a single film are shown in FIG. 19. In FIG. 19, the horizontal axis indicates a wavelength (nm) and the vertical axis indicates light emission intensity (arbitrary unit). Further, (a) indicates a light emission spectrum in a state of a single film (an excited wavelength: 325 nm) and (b) indicates a light emission spectrum in a state of being dissolved in a toluene solution (an excited wavelength: 380 nm). According to FIG. 19, it is found that light emission from PCzPCA1 has a peak at 435 nm in the state of a single film and has a peak at 443 nm in the toluene solution.

The obtained PCzPCA1 was deposited by an evaporation method, and ionization potential of the compound in a state of a thin film was 5.17 eV when being measured by using a photoelectron spectrometer (AC-2, manufactured by Riken Keiki Co., Ltd.). According to this result, it was found that a HOMO level was −5.17 eV. Also, a LUMO level was −1.82 eV when the LUMO level was obtained by setting a wavelength of an absorption edge at a longer wavelength side of the absorption spectrum of the compound in a state of a thin film ((a) in FIG. 18) as a energy gap (3.35 eV).

An oxidation reaction characteristic of PCzPCA1 was measured by a cyclic voltammetry (CV) measurement. An electrochemical analyzer (ALS model 600A, manufactured by BAS Inc.) was used for the measurement.

As for a solution used in the CV measurement, dehydrated dimethylformamide (DMF) was used as a solvent. Tetra-n-butylammonium perchlorate (n-Bu₄NClO₄), which is a supporting electrolyte, was dissolved so that the concentration of the tetra-n-butylammonium perchlorate was 100 mmol/L. Furthermore, PCzPCA1, which is an object to be measured, was dissolved therein and prepared so that the concentration thereof was 1 mmol/L. Also, a platinum electrode (PTE platinum electrode, manufactured by BAS Inc.) was used as a work electrode. A platinum electrode (VC-3 Pt counter electrode (5 cm), manufactured by BAS Inc.) was used as an auxiliary electrode. An Ag/Ag⁺ electrode (RE 5 nonaqueous reference electrode, manufactured by BAS Inc.) was used as a reference electrode.

An oxidation reaction characteristic was measured as follows: potential of the work electrode with respect to the reference electrode was changed from −0.16 to 0.5 V. Thereafter, a scan for changing from 0.5 to −0.16 V was set to be as one cycle, and 100 cycles were measured. It is to be noted that scan speed of the CV measurement was set to be 0.1 V/s.

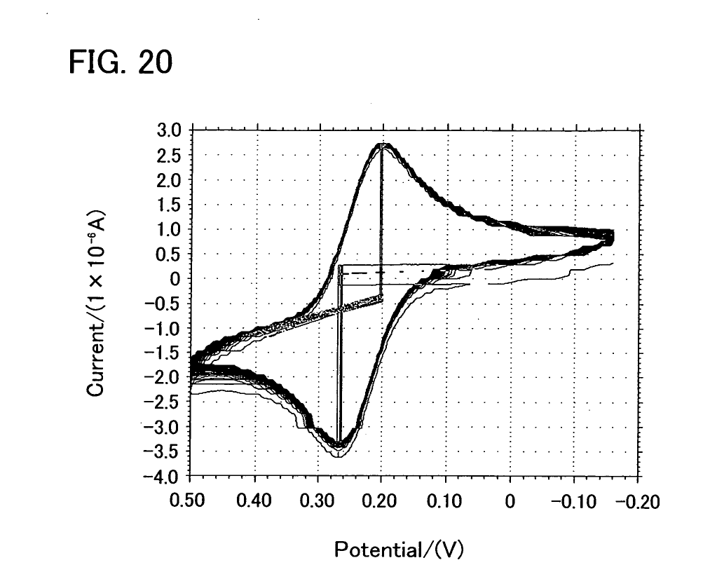

The examination result of the oxidation reaction characteristic of PCzPCA1 is shown in FIG. 20. In FIG. 20, the horizontal axis indicates potential (V) of the work electrode with respect to the reference electrode, and the vertical axis indicates a current value (1×10⁻⁶ A) flowing between the work electrode and the auxiliary electrode. According to FIG. 20, it was found that oxidation peak potential was 0.27 V (vs. Ag/Ag⁺ electrode). Although the scan was repeated for 100 cycles, changes in peak position or peak intensity of the CV curve were hardly seen as for the oxidation reaction. Accordingly, it was found that a carbazole derivative of the present invention is extremely stable to the oxidation reaction.

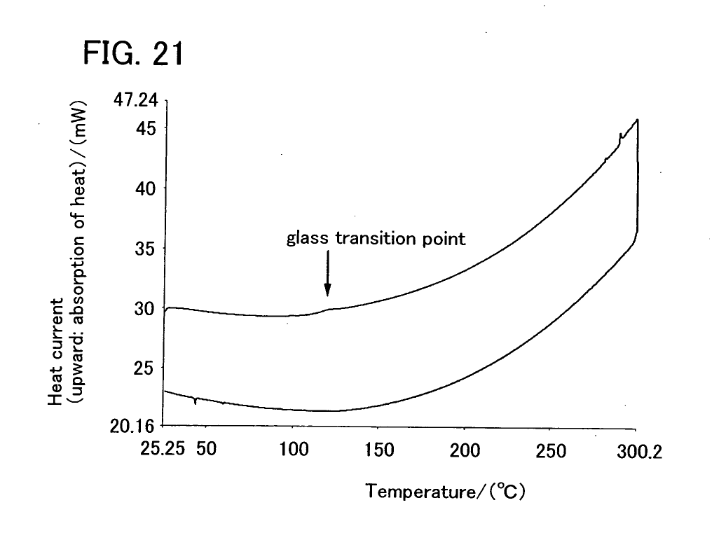

A glass transition temperature of the obtained compound PCzPCA1 was measured by using a differential scanning calorimetry (Pyris 1 DSC, manufactured by Perkin Elmer Co., Ltd.). The measurement result by DSC is shown in FIG. 21. According to the measurement result, it was found that the glass transition temperature of the obtained compound was 112° C. As described above, the obtained compound has the glass transition temperature as high as 112° C., and has favorable heat resistance. In addition, in FIG. 21, there is no peak showing crystallization of the obtained compound, and thus it was found that the obtained compound is hard to be crystallized.

### Synthesis Example 5

As one embodiment of the present invention using PCA which was synthesized in Synthesis Example 1, a synthesis of 3,6-bis[N-(9-phenylcarbazol-3-yl)-N-phenylamino]-9-phenylcarbazole (abbreviation: PCzPCA2) will be explained.

[Step 1]

A synthesis method of 3,6-diiodo-9-phenylcarbazole will be explained. A synthesis scheme of 3,6-diiodo-9-phenylcarbazole is shown in (k-1).

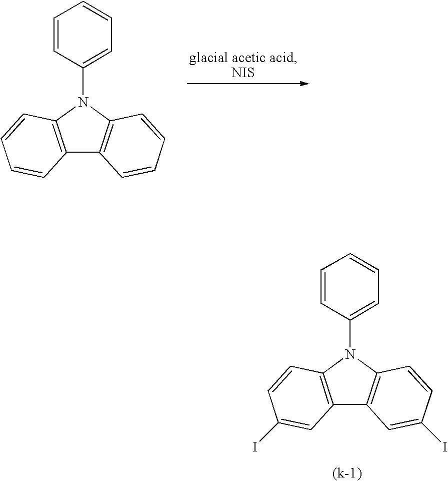

First, 24.3 g (100 mmol) of 9-phenylcarbazole was dissolved in 700 mL of glacial acetic acid, and 44.9 g (200 mmol) of N-iodosuccinimide was slowly added thereto. The mixture was stirred at a room temperature for about 20 hours. The generated precipitate was filtered, and the residue was washed with a saturated sodium hydrogen carbonate solution, water, and methanol and then dried. 47.0 g (yield: 95%) of 3,6-diiodo-9-phenylcarbazole, which was white powder, was obtained.

[Step 2]

A synthesis method of 3,6-bis[N-(9-phenylcarbazol-3-yl)-N-phenylamino]-9-phenylcarbazole (abbreviation: PCzPCA2) will be explained. A synthesis scheme of PCzPCA2 is shown in (k-2).

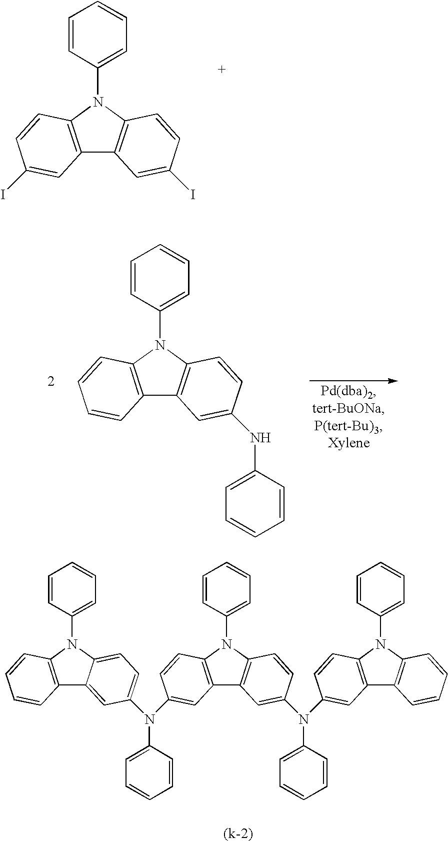

Under nitrogen, 30 mL of dehydrated xylene was added to a mixture of 2.5 g (5 mmol) of 3,6-diiodo-9-phenylcarbazole, 3.4g (10 mmol) of PCA, 30 mg (0.05 mmol) of bis(dibenzylideneacetone)palladium(0), 200 μL (0.5 mmol) of a hexane solution containing 49 wt % of tri-tert-butylphosphine, and 3.0 g (30 mmol) of sodium-tert-butoxide. This was stirred while heating at 90° C. under a nitrogen atmosphere for 6.5 hours. After the termination of the reaction, about 500 mL of hot toluene was added to the suspension and this suspension was filtered through Florisil®, alumina, and Celite. The obtained filtrate was condensed and its residue was separated by using silica gel column chromatography (toluene:hexane=1:1). This was condensed, and ethyl acetate-hexane was added to the obtained residue to conduct recrystallization. 2.5 g (yield: 55%) of 3,6-bis[N-(9-phenylcarbazol-3-yl)-N-phenylamino]-9-phenylcarbazole, which was cream-colored powder, was obtained.

¹H-NMR data is shown below.

¹H-NMR (300 MHz, DMSO-d₆); δ=6.74-6.80 (m, 6H), 7.08-7.64 (m, 33H), 7.94-8.04 (m, 6H),

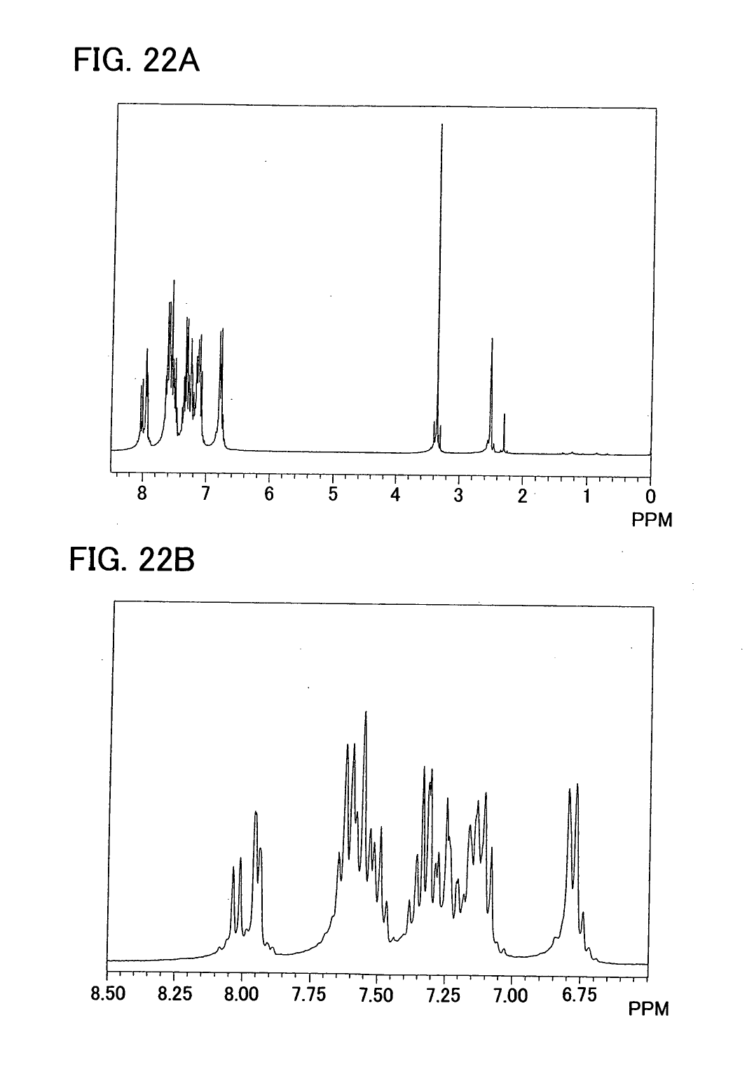

A ¹H-NMR chart is shown in FIG. 22A, and FIG. 22B is an enlarged chart of a portion of 6.50 to 8.50 ppm in FIG. 22A.

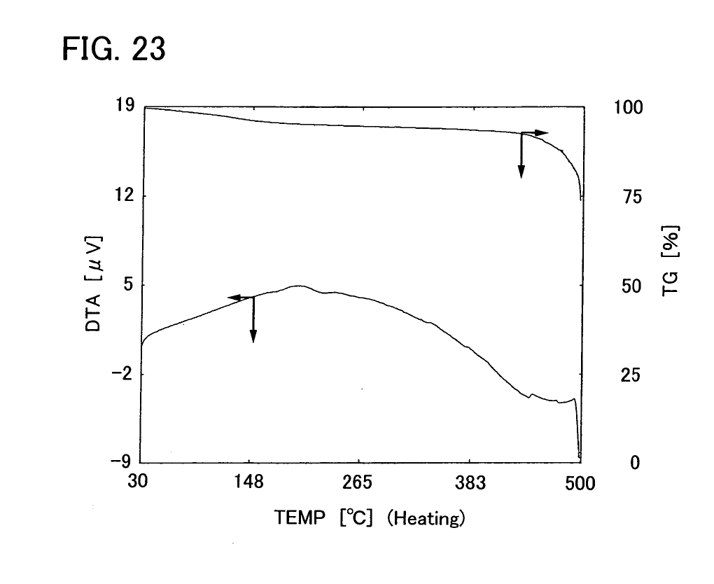

A thermogravimetry-differential thermal analysis (TG-DTA) of the obtained PCzPCA2 was performed. The result is shown in FIG. 23. In FIG. 23, the vertical axis on the left side indicates heat quantity (μV) and the vertical axis on the right side indicates gravity (%; gravity expressed assuming that gravity at the start of measurement is 100%). Furthermore, the lower horizontal axis indicates a temperature (° C.). A thermo-gravimetric/differential thermal analyzer (TG/DTA 320, manufactured by Seiko Instruments Inc.) was used for a measurement, and thermophysical properties were evaluated at a temperature rising rate of 10° C./min under a nitrogen atmosphere. As a result, from the relationship between the gravity and the temperature (thermogravimetry), the temperature T_{d}, at which the gravity becomes 95% or less with respect to the gravity at the start of the measurement, was 476° C. under normal pressure.

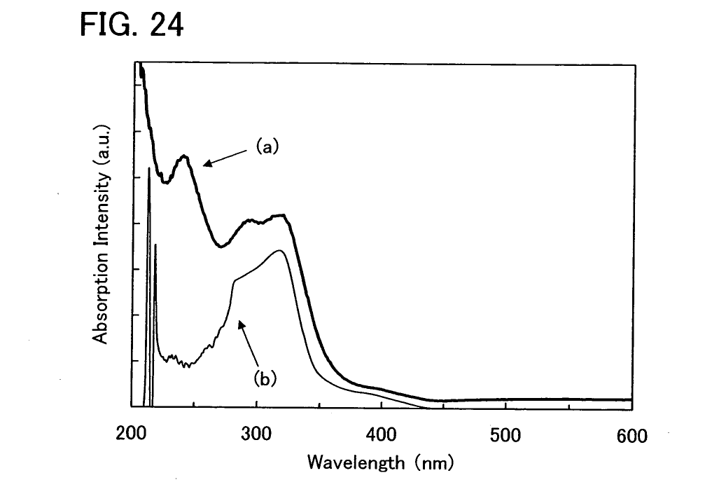

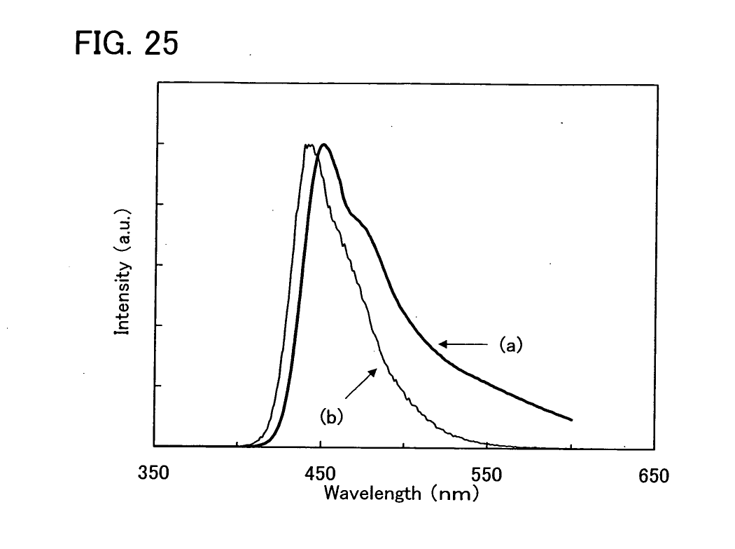

Absorption spectra of a toluene solution of PCzPCA2 and a thin film of PCzPCA2 are shown in FIG. 24. An ultraviolet-visible spectrophotometer (V-550, manufactured by JASCO Corporation) was used for a measurement. In FIG. 24, the horizontal axis indicates a wavelength (nm) and the vertical axis indicates absorption intensity (arbitrary unit). Also, (a) indicates an absorption spectrum in a state of a single film and (b) indicates an absorption spectrum in a state of being dissolved in a toluene solution. Light emission spectra of PCzPCA2 are shown in FIG. 25. In FIG. 25, the horizontal axis indicates a wavelength (nm) and the vertical axis indicates light emission intensity (arbitrary unit). Also, (a) indicates a light emission spectrum in a state of a single film (an excited wavelength: 320 nm) and (b) indicates a light emission spectrum in a state of being dissolved in a toluene solution (an excited wavelength: 325 nm). According to FIG. 25, light emission from PCzPCA2 has a peat at 449 nm in the state of a single film and has a peak at 442 in the toluene solution.

The obtained PCzPCA2 was deposited by an evaporation method, and ionization potential of the compound in a state of a thin film was 5.10 eV when being measured by using a photoelectron spectrometer (AC-2, manufactured by Riken Keiki Co., Ltd.). According to this result, it was found that a HOMO level was −5.10 eV. Also, a LUMO level was −1.75 eV when the LUMO level was obtained by setting wavelength of an absorption edge at a longer wavelength side of the absorption spectrum of the compound in a state of a thin film ((a) in FIG. 25) as an energy gap (3.35 eV).

In addition, an oxidation characteristic of PCzPCA2 was examined by a cyclic voltammetry (CV) measurement. It is to be noted that an electrochemical analyzer (ALS model 600A, manufactured by BAS Inc.) was used for the measurement.

As for a solution used in the CV measurement, dehydrated dimethylformamide (DMF) was used as a solvent. Tetra-n-butylammonium perchlorate (n-Bu₄NClO₄), which is a supporting electrolyte, was dissolved so that the concentration of the tetra-n-butylammonium perchlorate was 100 mmol/L. Furthermore, PCzPCA2, which is an object to be measured, was dissolved therein and prepared so that the concentration thereof was 1 mmol/L. Also, a platinum electrode (PTE platinum electrode, manufactured by BAS Inc.) was used as a work electrode. A platinum electrode (VC-3 Pt counter electrode (5 cm), manufactured by BAS Inc.) was used as an auxiliary electrode. An Ag/Ag⁺ electrode (RE 5 nonaqueous reference electrode, manufactured by BAS Inc.) was used as a reference electrode.

An oxidation reaction characteristic was measured as follows: potential of the work electrode with respect to the reference electrode was changed from −0.01 to 0.33 V. Thereafter, a scan for changing from 0.33 to −0.01 V was set to be as one cycle, and 100 cycles were measured. It is to be noted that scan speed of the CV measurement was set to be 0.1 V/s.

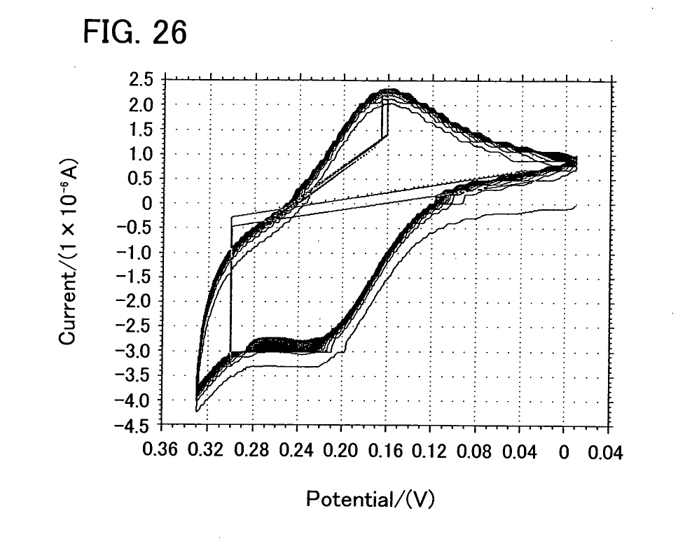

The examination result of the oxidation reaction characteristic of PCzPCA2 is shown in FIG. 26. In FIG. 26, the horizontal axis indicates potential (V) of the work electrode with respect to the reference electrode, and the vertical axis indicates a current value (1×10⁻⁶ A) flowing between the work electrode and the auxiliary electrode. According to FIG. 26, it was found that oxidation peak potential was 0.22 V (vs. Ag/Ag⁺ electrode). Although the scan was repeated for 100 cycles, changes in peak position or peak intensity of the CV curve were hardly seen as for the oxidation reaction. Accordingly, it was found that a carbazole derivative of the present invention is extremely stable to the oxidation reaction.

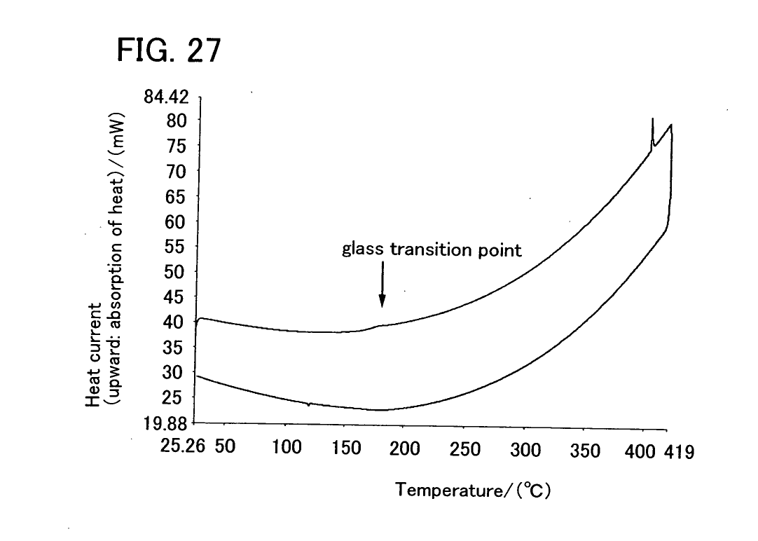

A glass transition temperature of the obtained compound PCzPCA2 was examined by using a differential scanning calorimetry (Pyris 1 DSC, manufactured by Perkin Elmer Co., Ltd.). The measurement result by DSC is shown in FIG. 27. According to the measurement result, it was found that the glass transition temperature of the obtained compound was 168° C. As described above, the obtained compound has the glass transition temperature as high as 168° C., and has favorable heat resistance. In addition, in FIG. 27, there is no peak showing crystallization of the obtained compound, and thus it was found that the obtained compound is hard to be crystallized.

### Embodiment 3

As one embodiment of the present invention using PCN which was synthesized in Synthesis Example 2, a synthesis of 3-[N-(1-naphthyl)-N-(9-phenylcarbazol-3-yl)amino]-9-phenylcarbazole (abbreviation: PCzPCN1) will be explained.

A synthesis scheme of PCzPCN1 is shown in (l-1).

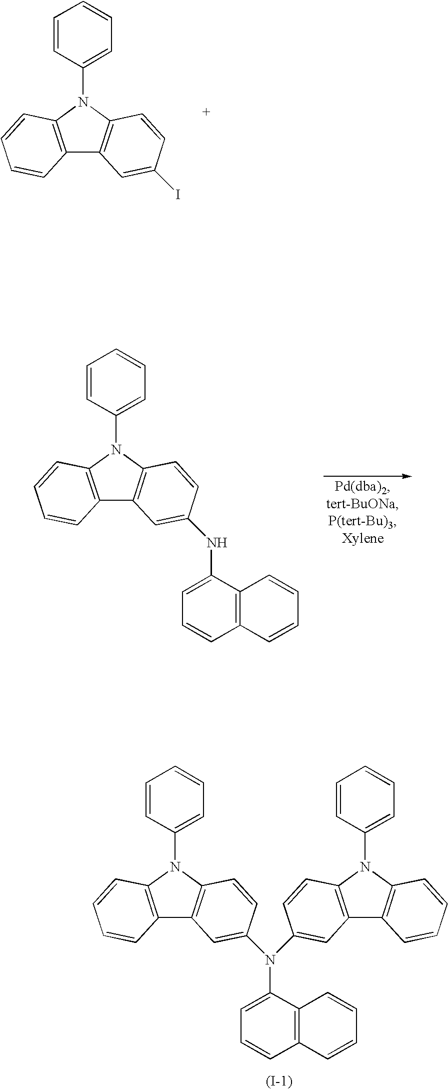

Under nitrogen, 7 mL of dehydrated xylene was added to a mixture of 1.8 g (5 mmol) of 3-iodo-9-phenylcarbazole, 2.5 g (6.6 mmol) of PCN, 30 mg (0.05 mmol) of bis(dibenzylideneacetone)palladium(0), 200 μL (0.5 mmol) of a hexane solution containing 49 wt % of tri-tert-butylphosphine, and 700 mg (7 mmol) of sodium-tert-butoxide. This was stirred while heating at 90° C. under a nitrogen atmosphere for 4.5 hours. After the termination of the reaction, about 500 mL of hot toluene was added to the suspension and the suspension was filtered through Florisil®, alumina, and Celite. The obtained filtrate was condensed and the residue was separated by silica gel column chromatography (toluene:hexane=1:1). The obtained solid was recrystallized with ethyl acetate-hexane. 2.1 g (yield: 62%) of PCzPCN1, which was yellow powder, was obtained.

NMR data is shown below.

¹H-NMR (300 MHz, DMSO-d₆); δ=7.04-7.65 (m, 24H), 7.78 (d, J=8.4 Hz, 1H), 7.82 (d, J=2.1 Hz, 2H), 7.88 (d, J=7.8 Hz, 2H), 7.95 (d, J=8.4 Hz, 1H), 8.10 (d, J=9.0 Hz, 1H)

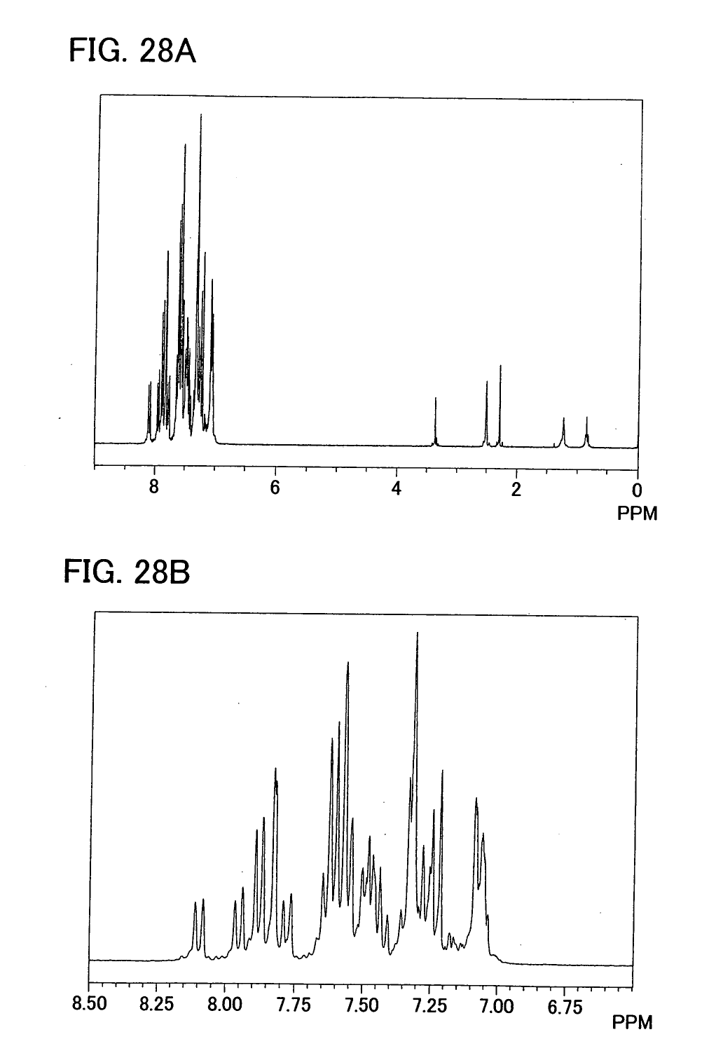

A ¹H-NMR chart is shown in FIG. 28A, and FIG. 28B is an enlarged chart of a portion of 6.50 to 8.50 ppm in FIG. 28A.

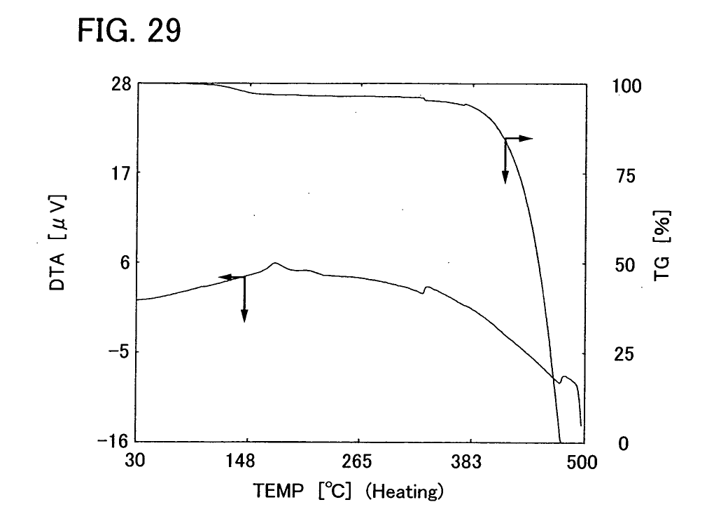

A thermogravimetry-differential thermal analysis (TG-DTA) of the obtained PCzPCN1 was performed in the same manner as in Embodiments 1 and 2. The result is shown in FIG. 29. In FIG. 29, the vertical axis on the left side indicates heat quantity (μV) and the vertical axis on the right side indicates gravity (%; gravity expressed assuming that gravity at the start of measurement is 100%). Furthermore, the lower horizontal axis indicates a temperature (° C.). A thermo-gravimetric/differential thermal analyzer (320, manufactured by Seiko Instruments Inc.) was used for a measurement, and thermophysical properties were evaluated at a temperature rising rate of 10° C./min under a nitrogen atmosphere. As a result, from the relationship between the gravity and the temperature (thermogravimetry), the temperature T_{d}, at which the gravity becomes 95% or less with respect to the gravity at the start of the measurement, was 400° C. under normal pressure.

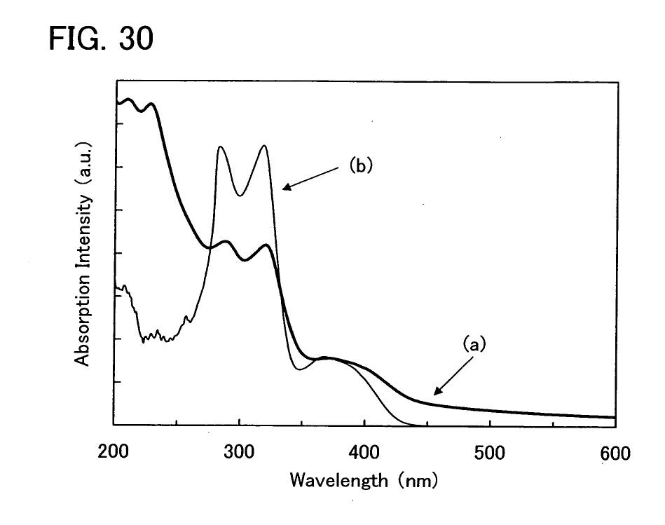

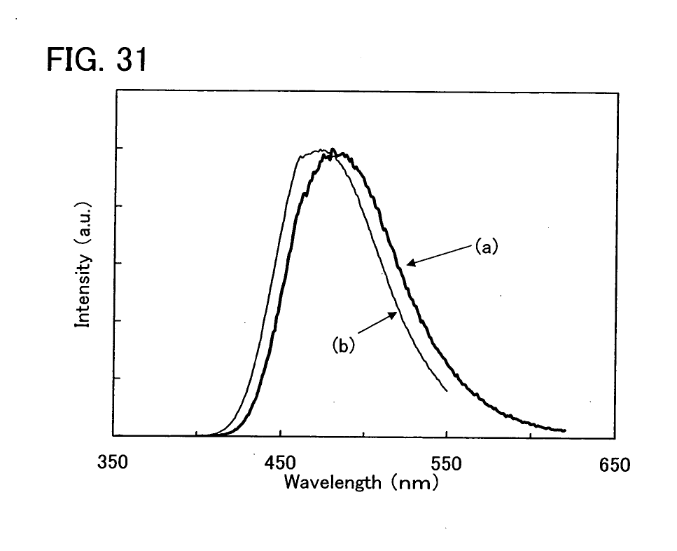

Absorption spectra of a toluene solution of PCzPCN1 and a thin film of PCzPCN1 are shown in FIG. 30. An ultraviolet-visible spectrophotometer (V-550, manufactured by JASCO Corporation) was used for a measurement. In FIG. 30, the horizontal axis indicates a wavelength (nm) and the vertical axis indicates absorption intensity (arbitrary unit). A light emission spectrum of the toluene solution of PCzPCN1 is shown in FIG. 31. In FIG. 31, the horizontal axis indicates a wavelength (nm) and the vertical axis indicates light emission intensity (arbitrary unit). In addition, (a) indicates a light emission spectrum in a state of a single film (an excited wavelength: 320 nm) and (b) indicates a light emission spectrum in a state of being dissolved in a toluene solution (an excited wavelength: 320 nm). According to FIG. 31, it was found that light emission from PCzPCN1 has a peak at 485 nm in the state of a single film, and has a peak at 475 nm in the toluene solution.

The obtained PCzPCN1 was deposited by an evaporation method, and ionization potential of the compound in a state of a thin film was 5.15 eV when being measured by using a photoelectron spectrometer (AC-2, manufactured by Riken Keiki Co., Ltd.). According to this result, it was found that a HOMO level was −5.15 eV. Also, a LUMO level was −2.33 eV when the LUMO level was obtained by setting a wavelength of an absorption edge at a longer wavelength side of the absorption spectrum of the compound in a state of a thin film ((a) in FIG. 31) as an energy gap (2.82 eV).

In addition, an oxidation characteristic of PCzPCN1 was measured by a cyclic voltammetry (CV) measurement. It is to be noted that an electrochemical analyzer (ALS model 600A, manufactured by BAS Inc.) was used for the measurement.

As for a solution used in the CV measurement, dehydrated dimethylformamide (DMF) was used as a solvent. Tetra-n-butylammonium perchlorate (n-Bu₄NClO₄), which is a supporting electrolyte, was dissolved so that the concentration of the tetra-n-butylammonium perchlorate was 100 mmol/L. Furthermore, PCzPCN1, which is an object to be measured, was dissolved therein and prepared so that the concentration thereof was 1 mmol/L. Also, a platinum electrode (PTE platinum electrode, manufactured by BAS Inc.) was used as a work electrode. A platinum electrode (VC-3 Pt counter electrode (5 cm), manufactured by BAS Inc.) was used as an auxiliary electrode. An Ag/Ag⁺ electrode (RE 5 nonaqueous reference electrode, manufactured by BAS Inc.) was used as a reference electrode.

An oxidation reaction characteristic was measured as follows: potential of the work electrode with respect to the reference electrode was changed from −0.20 to 0.50 V. Thereafter, a scan for changing from −0.20 to −0.50 V was set to be as one cycle, and 100 cycles were measured. It is to be noted that scan speed of the CV measurement was set to be 0.1 V/s.

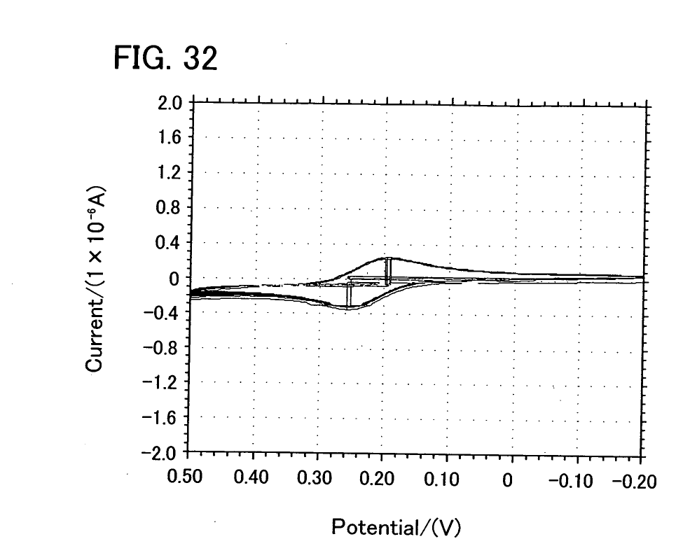

The examination result of the oxidation reaction characteristic of PCzPCN1 is shown in FIG. 32. In FIG. 32, the horizontal axis indicates potential (V) of the work electrode with respect to the reference electrode, and the vertical axis indicates a current value (1×10⁻⁶ A) flowing between the work electrode and the auxiliary electrode. According to FIG. 32, it was found that oxidation peak potential was 0.25 V (vs. Ag/Ag⁺ electrode). Although the scan was repeated for 100 cycles, changes in peak position or peak intensity of the CV curve are hardly seen as for the oxidation reaction. Accordingly, it was found that a carbazole derivative of the present invention is extremely stable to the oxidation reaction.

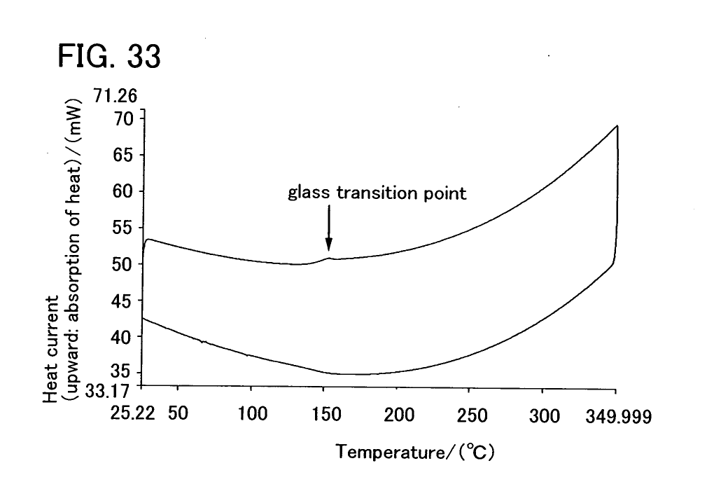

A glass transition temperature of the obtained compound PCzPCN1 was examined by using a differential scanning calorimetry (Pyris 1 DSC, manufactured by Perkin Elmer Co., Ltd.). The measurement result by a DSC is shown in FIG. 33. According to the measurement result, it was found that the glass transition temperature of the obtained compound was 142° C. As described above, the obtained compound has the glass transition temperature as high as 142° C., and has favorable heat resistance. In addition, in FIG. 33, there is no peak showing crystallization of the obtained compound, and thus it was found that the obtained compound is hard to be crystallized.

### Embodiment 4

In this embodiment, a manufacturing method of a light-emitting element using PCABPA synthesized in Synthesis Example 3 as a light-emitting substance and an operation characteristic thereof will be explained.

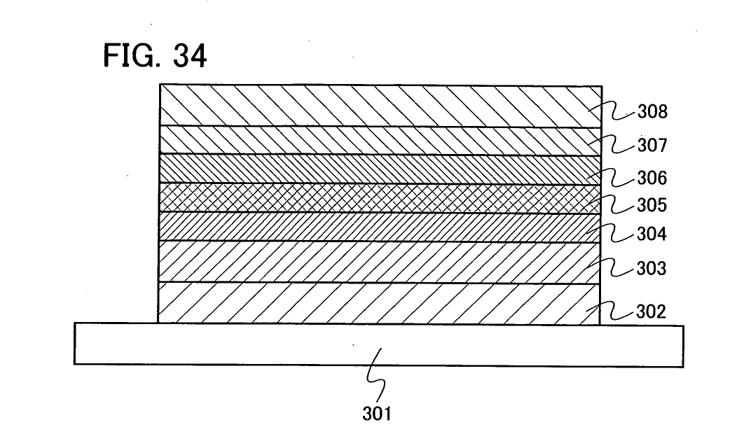

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using copper phthalocyanine by an evaporation method. The first layer 303 was formed so as to be 20 nm thick. This first layer 303 serves as a hole injecting layer when a light-emitting element is operated.

Then, a second layer 304 was formed over the first layer 303 by using NPB by an evaporation method. The second layer 304 was formed so as to be 40 nm thick. This second layer 304 serves as a hole transporting layer when the light-emitting element is operated.

Next, a third layer 305 containing t-BuDNA and PCABPA was formed over the second layer 304 by a co-evaporation method. The third layer 305 was formed so that its thickness was 40 nm and a mass ratio of t-BuDNA to PCABPA was 1:0.05. Accordingly, PCABPA is in such a state that PCABPA is dispersed in a layer containing t-BuDNA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm thick. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

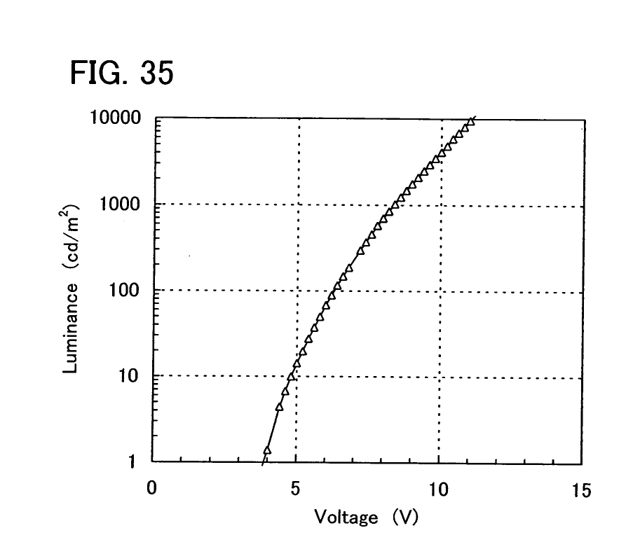

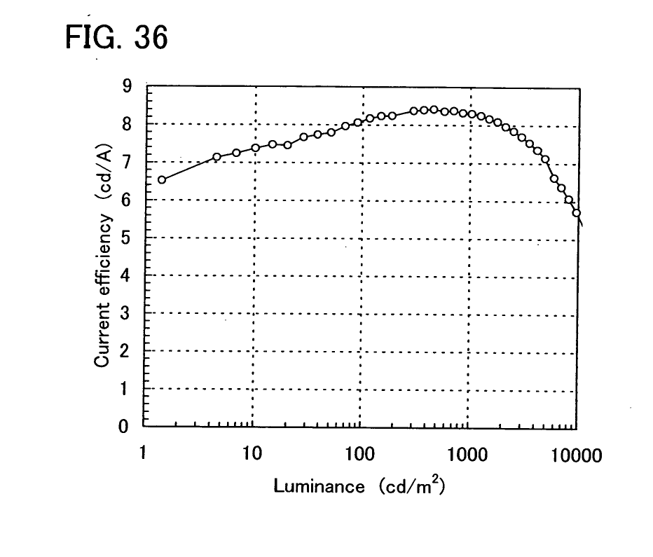

The measurement results are shown in FIGS. 35 and 36. FIG. 35 shows the measurement result of a voltage-luminance characteristic, and FIG. 36 shows the measurement result of a luminance-current efficiency characteristic. In FIG. 35, the horizontal axis indicates voltage (V) and the vertical axis indicates luminance (cd/m²). In FIG. 36, the horizontal axis indicates luminance (cd/m²) and the vertical axis indicates current efficiency (cd/A).

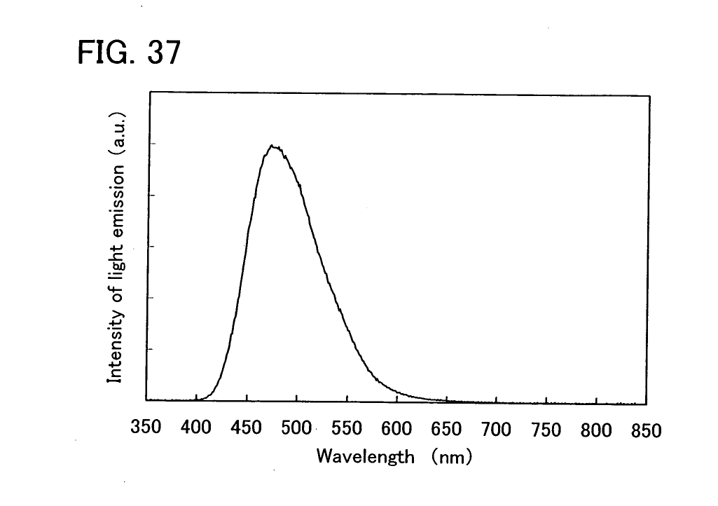

A light emission spectrum of the light-emitting element manufactured in this embodiment is shown in FIG. 37. In FIG. 37, the horizontal axis indicates a wavelength (nm) and the vertical axis indicates intensity (arbitrary unit). According to FIG. 37, it was found that the light-emitting element of this embodiment had a peak of the light emission spectrum at 477 nm and exhibited blue light emission. Furthermore, it was found that a CIE chromaticity coordinate was (x, y)=(0.16, 0.28) and the light-emitting element of this embodiment exhibited blue light emission with favorable color purity.

### Embodiment 5

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using copper phthalocyanine by an evaporation method. The first layer 303 was formed so as to be 20 nm thick. This first layer 303 serves as a hole injecting layer when a light-emitting element is operated.

Then, a second layer 304 was formed over the first layer 303 by using 4,4′-bis[N-(4-biphenylyl)-N-phenylamino]biphenyl (abbreviation: BBPB) by an evaporation method. The second layer 304 was formed so as to be 40 nm thick. This second layer 304 serves as a hole transporting layer when the light-emitting element is operated.

Next, a third layer 305 containing t-BuDNA and PCABPA was formed over the second layer 304 by a co-evaporation method. The third layer 305 was formed so that its thickness was 40 nm and a mass ratio of t-BuDNA to PCABPA was 1:0.05. Accordingly, PCABPA is in such a state that PCABPA is dispersed in a layer containing t-BuDNA as its main component. This third layer 305 serves as a light-emitting layer when the light-emitting element is operated. Further, PCABPA serves as a light-emitting substance.

Then, a fourth layer 306 was formed over the third layer 305 by using Alq3 by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm thick. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

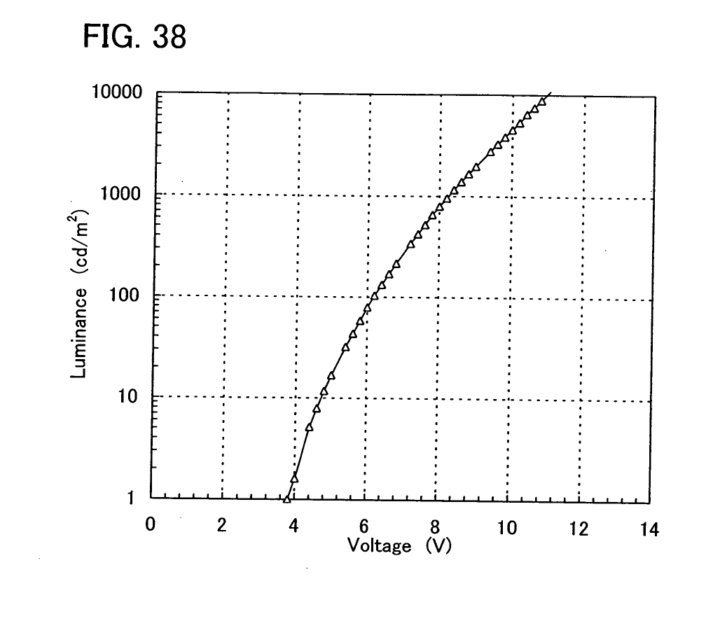

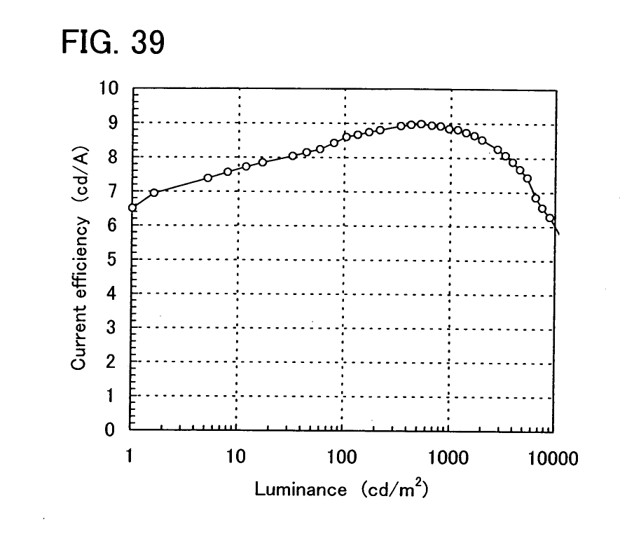

The measurement results are shown in FIGS. 38 and 39. FIG. 38 shows the measurement result of a voltage-luminance characteristic, and FIG. 39 shows the measurement result of a luminance-current efficiency characteristic. In FIG. 38, the horizontal axis indicates voltage (V) and the vertical axis indicates luminance (cd/m²). In FIG. 39, the horizontal axis indicates luminance (cd/m²) and the vertical axis indicates current efficiency (cd/A).

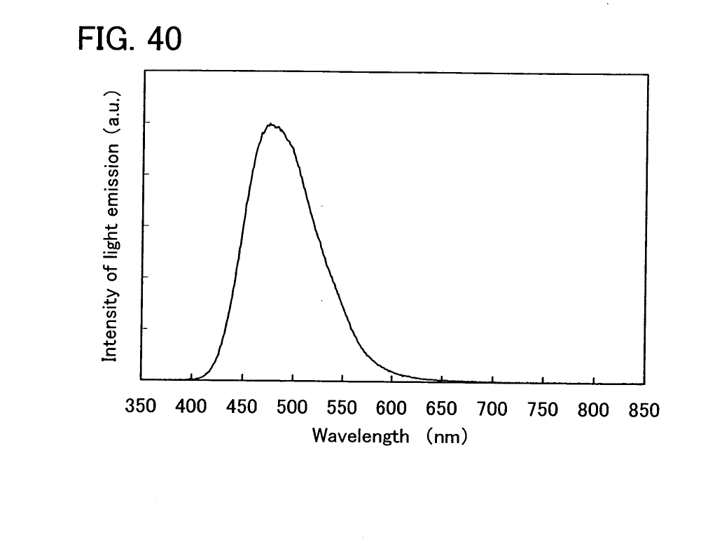

A light emission spectrum of the light-emitting element manufactured in this embodiment is shown in FIG. 40. In FIG. 40, the horizontal axis indicates a wavelength (nm) and the vertical axis indicates intensity (arbitrary unit). According to FIG. 40, it was found that the light-emitting element of this embodiment had a peak of the light emission spectrum at 479 nm and exhibited blue light emission. Furthermore, it was found that a CIE chromaticity coordinate was (x, y)=(0.16, 0.29) and the light-emitting element of this embodiment exhibited blue light emission with favorable color purity.

### Embodiment 6

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using copper phthalocyanine by an evaporation method. The first layer 303 was formed so as to be 20 nm thick. This first layer 303 serves as a hole injecting layer when a light-emitting element is operated.

Then, a second layer 304 was formed over the first layer 303 by using N,N′-bis(spiro-9,9′-bifluorene-2-yl)-N,N′-diphenylbenzidine (abbreviation: BSPB) by an evaporation method. The second layer 304 was formed so as to be 40 nm thick. This second layer 304 serves as a hole transporting layer when the light-emitting element is operated.

Next, a third layer 305 containing t-BuDNA and PCABPA was formed over the second layer 304 by a co-evaporation method. The third layer 305 was formed so that its thickness was 40 nm and a mass ratio of t-BuDNA to PCABPA was 1:0.05. Accordingly, PCABPA is in such a state that PCABPA is dispersed in a layer containing t-BuDNA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm thick. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

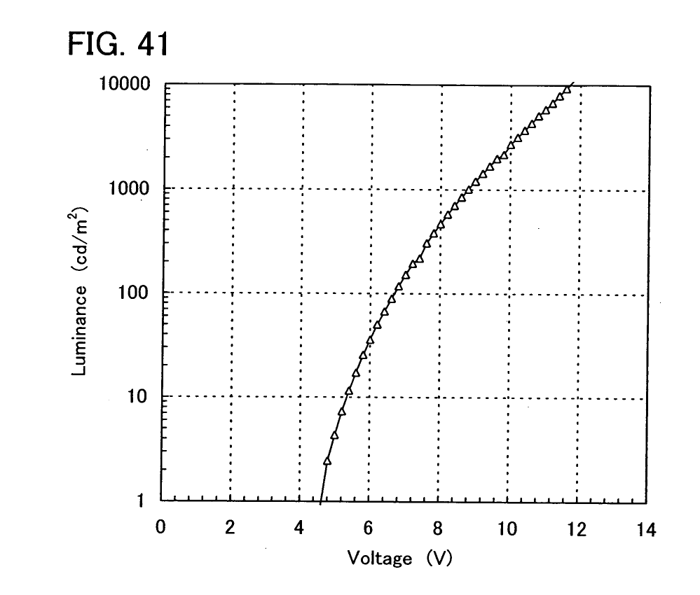

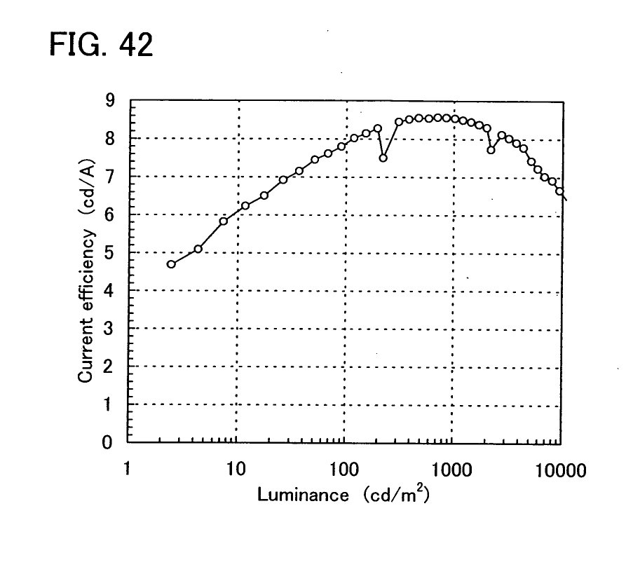

The measurement results are shown in FIGS. 41 and 42. FIG. 41 shows the measurement result of a voltage-luminance characteristic, and FIG. 42 shows the measurement result of a luminance-current efficiency characteristic. In FIG. 41, the horizontal axis indicates voltage (V) and the vertical axis indicates luminance (cd/m²). In FIG. 42, the horizontal axis indicates luminance (cd/m²) and the vertical axis indicates current efficiency (cd/A).

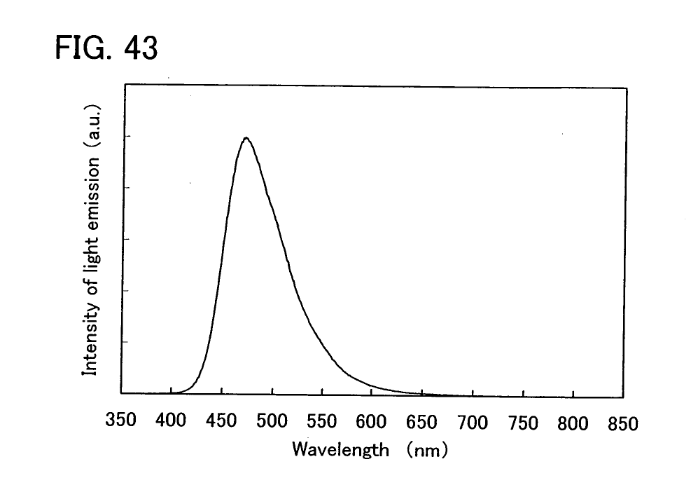

A light emission spectrum of the light-emitting element manufactured in this embodiment is shown in FIG. 43. In FIG. 43, the horizontal axis indicates a wavelength (nm) and the vertical axis indicates intensity (arbitrary unit). According to FIG. 43, it was found that the light-emitting element of this embodiment had a peak of the light emission spectrum at 474 nm and exhibited blue light emission. Furthermore, it was found that a CIE chromaticity coordinate was (x, y)=(0.16, 0.25) and the light-emitting element of this embodiment exhibited blue light emission with favorable color purity.

### Embodiment 7

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using DNTPD by an evaporation method. The first layer 303 was formed so as to be 50 nm thick. This first layer 303 serves as a hole injecting layer when a light-emitting element is operated.

Then, a second layer 304 was formed over the first layer 303 by using NPB by an evaporation method. The second layer 304 was formed so as to be 10 nm thick. This second layer 304 serves as a hole transporting layer when the light-emitting element is operated.

Next, a third layer 305 containing CzPA and PCABPA was formed over the second layer 304 by a co-evaporation method. The third layer 305 was formed so that its thickness was 40 nm and a mass ratio of CzPA to PCABPA was 1:0.05. Accordingly, PCABPA is in such a state that PCABPA is dispersed in a layer containing CzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm thick. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

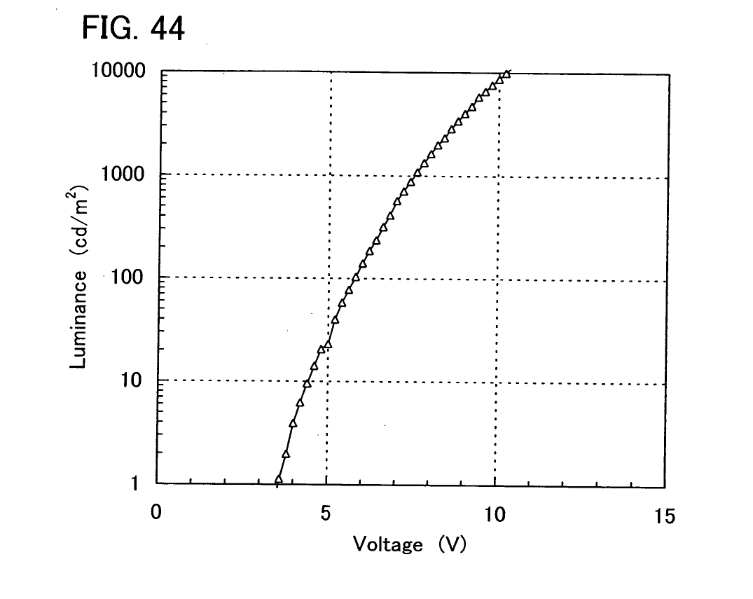

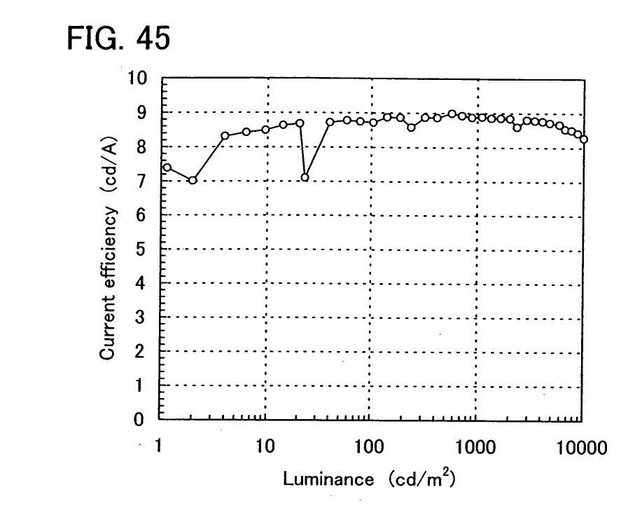

The measurement results are shown in FIGS. 44 and 45. FIG. 44 shows the measurement result of a voltage-luminance characteristic, and FIG. 45 shows the measurement result of a luminance-current efficiency characteristic. In FIG. 44, the horizontal axis indicates voltage (V) and the vertical axis indicates luminance (cd/m²). In FIG. 45, the horizontal axis indicates luminance (cd/m²) and the vertical axis indicates current efficiency (cd/A).

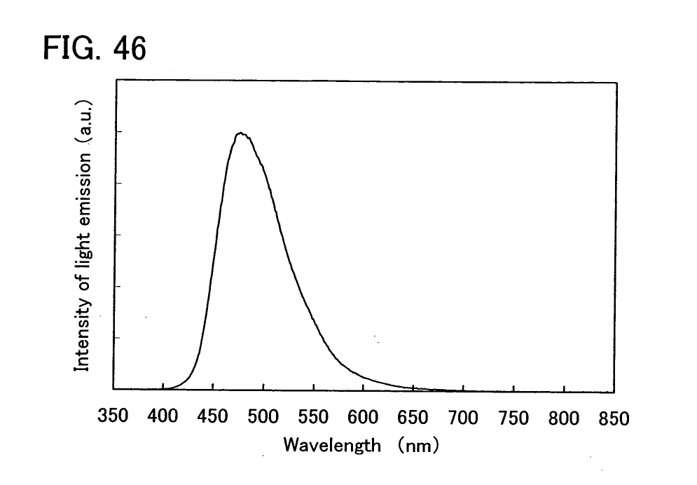

A light emission spectrum of the light-emitting element manufactured in this embodiment is shown in FIG. 46. In FIG. 46, the horizontal axis indicates wavelength (nm) and the vertical axis indicates intensity (arbitrary unit). According to FIG. 46, it was found that the light-emitting element of this embodiment had a peak of the light emission spectrum at 478 nm and exhibited blue light emission. Furthermore, it was found that a CIE chromaticity coordinate was (x, y)=(0.16, 0.28) and the light-emitting element of this embodiment exhibited blue light emission with favorable color purity.

### Embodiment 8

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using DNTPD by an evaporation method. The first layer 303 was formed so as to be 50 nm thick. This first layer 303 serves as a hole injecting layer when a light-emitting element is operated.

Then, a second layer 304 was formed over the first layer 303 by using NPB by an evaporation method. The second layer 304 was formed so as to be 10 nm thick. This second layer 304 serves as a hole transporting layer when the light-emitting element is operated.

Next, a third layer 305 containing CzPA and PCABPA was formed over the second layer 304 by a co-evaporation method. The third layer 305 was formed so that its thickness was 40 nm and a mass ratio of CzPA to PCABPA was 1:0.04. Accordingly, PCABPA is in such a state that PCABPA is dispersed in a layer containing CzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

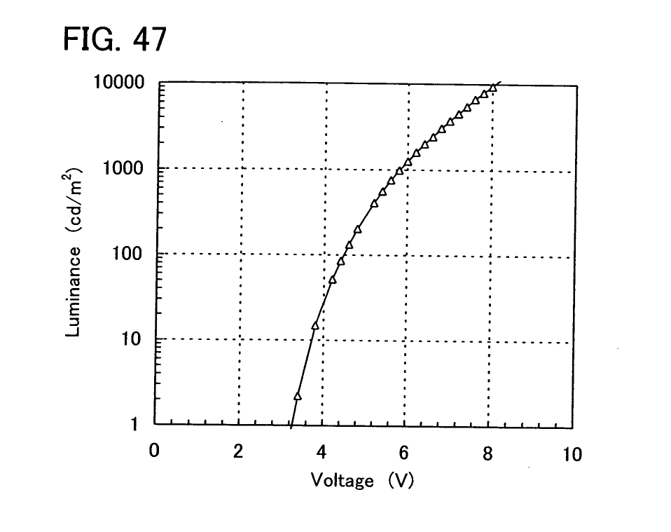

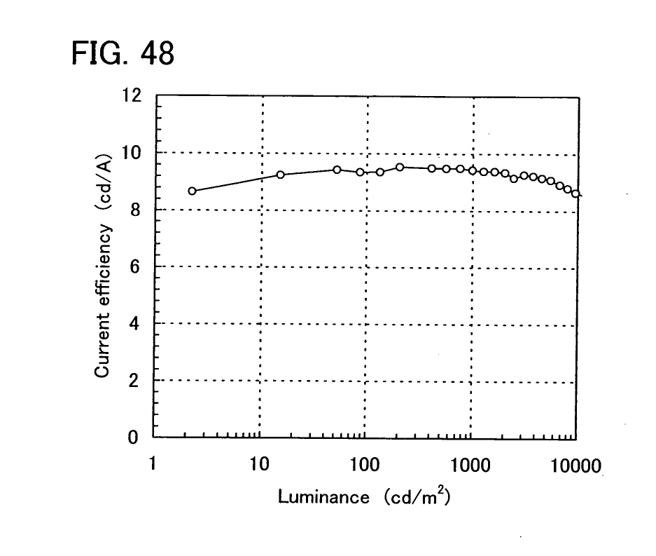

The measurement results are shown in FIGS. 47 and 48. FIG. 47 shows the measurement result of a voltage-luminance characteristic, and FIG. 48 shows the measurement result of a luminance-current efficiency characteristic. In FIG. 47, the horizontal axis indicates voltage (V) and the vertical axis indicates luminance (cd/m²). In FIG. 48, the horizontal axis indicates luminance (cd/m²) and the vertical axis indicates current efficiency (cd/A).

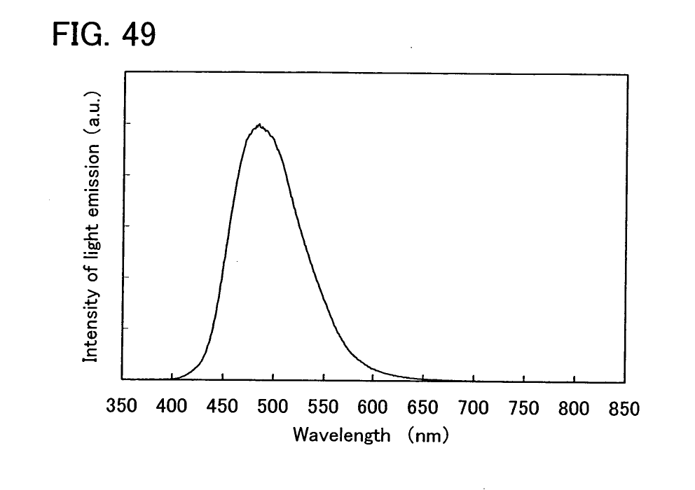

A light emission spectrum of the light-emitting element manufactured in this embodiment is shown in FIG. 49. In FIG. 49, the horizontal axis indicates a wavelength (nm) and the vertical axis indicates intensity (arbitrary unit). According to FIG. 49, it was found that the light-emitting element of this embodiment had a peak of the light emission spectrum at 487 nm and exhibited blue light emission. Furthermore, it was found that a CIE chromaticity coordinate was (x, y)=(0.17, 0.32) and the light-emitting element of this embodiment exhibited blue light emission with favorable color purity.

### Embodiment 9

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using DNTPD by an evaporation method. The first layer 303 was formed so as to be 50 nm thick. This first layer 303 serves as a hole injecting layer when a light-emitting element is operated.

Then, a second layer 304 was formed over the first layer 303 by using NPB by an evaporation method. The second layer 304 was formed so as to be 10 nm thick. This second layer 304 serves as a hole transporting layer when the light-emitting element is operated.

Next, a third layer 305 containing DPCzPA and PCABPA was formed over the second layer 304 by a co-evaporation method. The third layer 305 was formed so that its thickness was 40 nm and a mass ratio of DPCzBA to PCABPA was 1:0.04. Accordingly, PCABPA is in such a state that PCABPA is dispersed in a layer containing DPCzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

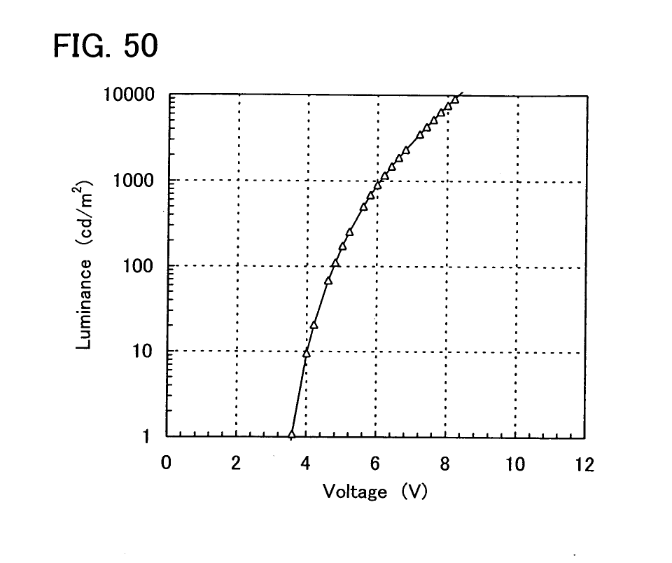

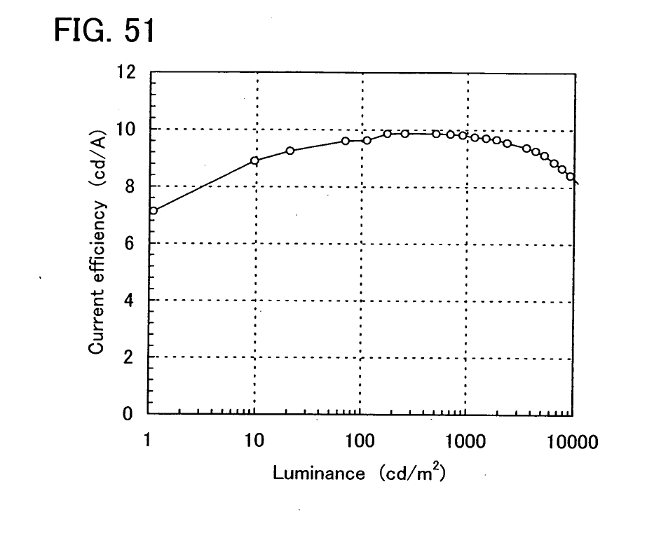

The measurement results are shown in FIGS. 50 and 51. FIG. 50 shows the measurement result of a voltage-luminance characteristic, and FIG. 51 shows the measurement result of a luminance-current efficiency characteristic. In FIG. 50, the horizontal axis indicates voltage (V) and the vertical axis indicates luminance (cd/m²). In FIG. 51, the horizontal axis indicates luminance (cd/m²) and the vertical axis indicates current efficiency (cd/A).

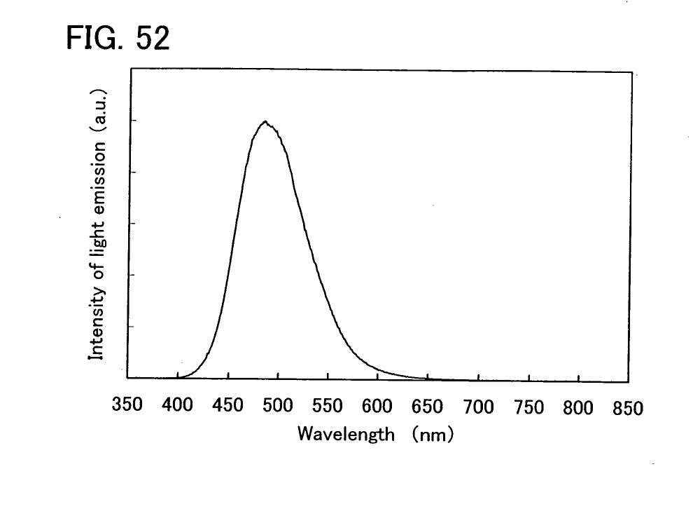

A light emission spectrum of the light-emitting element manufactured in this embodiment is shown in FIG. 52. In FIG. 52, the horizontal axis indicates a wavelength (nm) and the vertical axis indicates intensity (arbitrary unit). According to FIG. 52, it was found that the light-emitting element of this embodiment had a peak of the light emission spectrum at 487 nm and exhibited blue light emission. Furthermore, it was found that a CIE chromaticity coordinate was (x, y)=(0.17, 0.32) and the light-emitting element of this embodiment exhibited blue light emission with favorable color purity.

### Embodiment 10

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using DNTPD by an evaporation method. The first layer 303 was formed so as to be 50 nm thick. This first layer 303 serves as a hole injecting layer when a light-emitting element is operated.

Then, a second layer 304 was formed over the first layer 303 by using NPB by an evaporation method. The second layer 304 was formed so as to be 10 nm thick. This second layer 304 serves as a hole transporting layer when the light-emitting element is operated.

Next, a third layer 305 containing t-BuDNA and PCABPA was formed over the second layer 304 by a co-evaporation method. The third layer 305 was formed so that its thickness was 40 nm and a mass ratio of t-BuDNA to PCABPA was 1:0.04. Accordingly, PCABPA is in such a state that PCABPA is dispersed in a layer containing t-BuDNA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

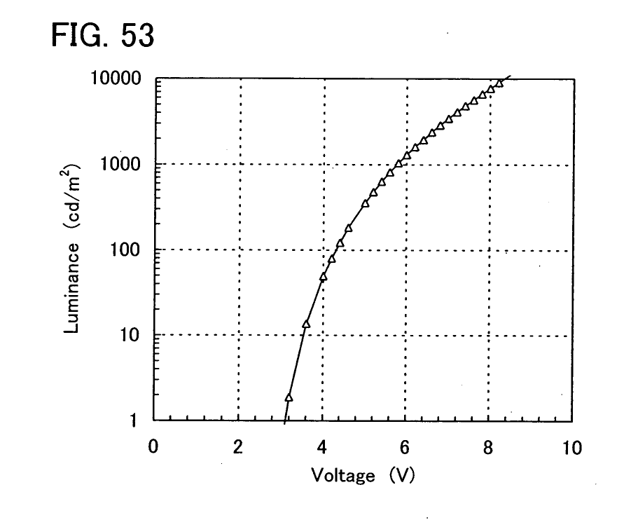

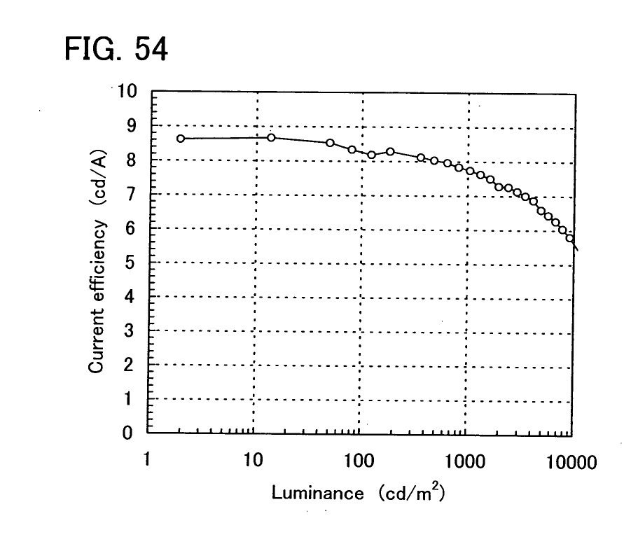

The measurement results are shown in FIGS. 53 and 54. FIG. 53 shows the measurement result of a voltage-luminance characteristic, and FIG. 54 shows the measurement result of a luminance-current efficiency characteristic. In FIG. 53, the horizontal axis indicates voltage (V) and the vertical axis indicates luminance (cd/m²). In FIG. 54, the horizontal axis indicates luminance (cd/m²) and the vertical axis indicates current efficiency (cd/A).

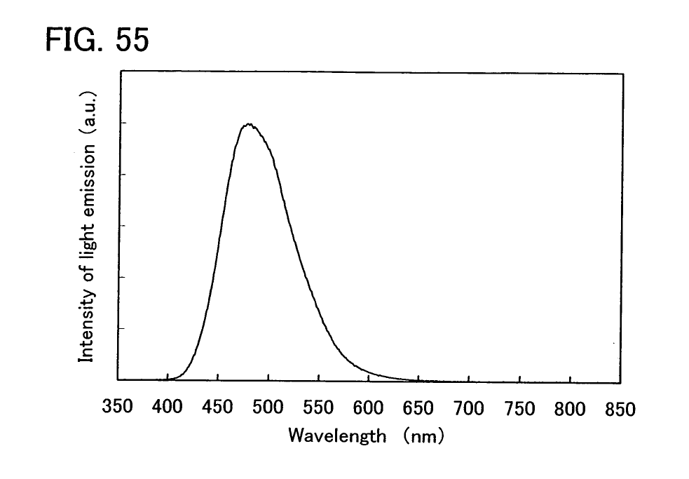

A light emission spectrum of the light-emitting element manufactured in this embodiment is shown in FIG. 55. In FIG. 55, the horizontal axis indicates a wavelength (nm) and the vertical axis indicates intensity (arbitrary unit). According to FIG. 55, it was found that the light-emitting element of this embodiment had a peak of the light emission spectrum at 482 nm and exhibited blue light emission. Furthermore, it was found that a CIE chromaticity coordinate was (x, y)=(0.16, 0.29) and the light-emitting element of this embodiment exhibited blue light emission with favorable color purity.

### Embodiment 11

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 24, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using CuPc by an evaporation method. The first layer 303 was formed so as to be 20 nm thick. This first layer 303 serves as a hole injecting layer when a light-emitting element is operated.

Then, a second layer 304 was formed over the first layer 303 by using NPB by an evaporation method. The second layer 304 was formed so as to be 40 nm thick. This second layer 304 serves as a hole transporting layer when the light-emitting element is operated.

Next, a third layer 305 containing CzPA and PCABPA was formed over the second layer 304 by a co-evaporation method. The third layer 305 was formed so that its thickness was 40 nm and a mass ratio of CzPA to PCABPA was 1:0.04. Accordingly, PCABPA is in such a state that PCABPA is dispersed in a layer containing CzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

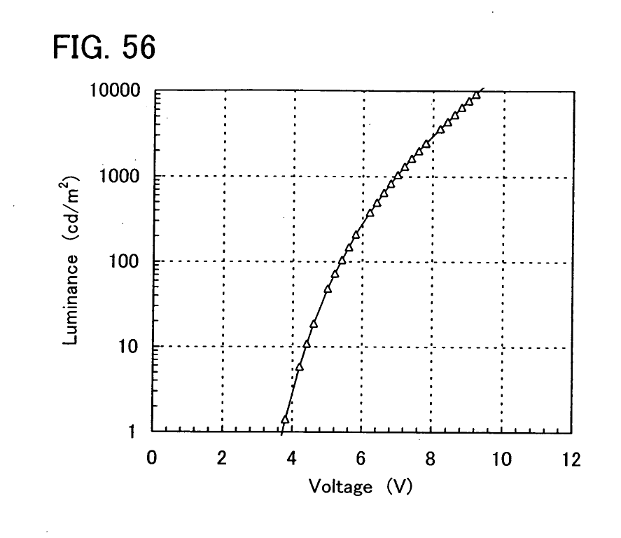

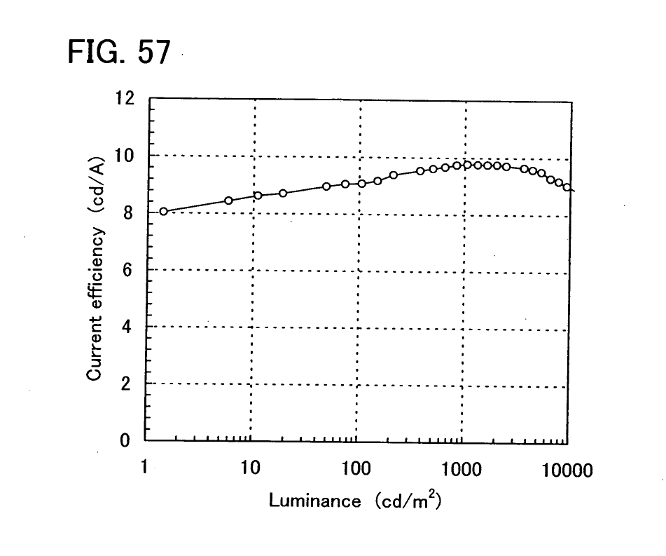

The measurement results are shown in FIGS. 56 and 57. FIG. 56 shows the measurement result of a voltage-luminance characteristic, and FIG. 57 shows the measurement result of a luminance-current efficiency characteristic. In FIG. 56, the horizontal axis indicates voltage (V) and the vertical axis indicates luminance (cd/m²). In FIG. 57, the horizontal axis indicates luminance (cd/m²) and the vertical axis indicates current efficiency (cd/A).

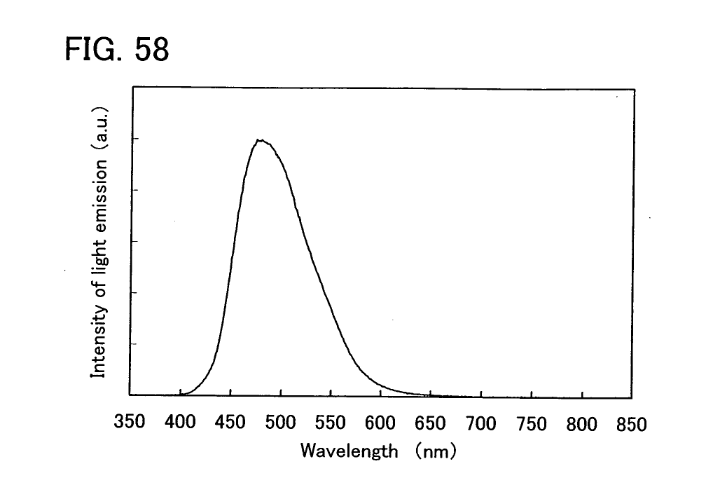

A light emission spectrum of the light-emitting element manufactured in this embodiment is shown in FIG. 58. In FIG. 58, the horizontal axis indicates a wavelength (nm) and the vertical axis indicates intensity (arbitrary unit). According to FIG. 58, it was found that the light-emitting element of this embodiment had a peak of the light emission spectrum at 481 nm and exhibited blue light emission. Furthermore, it was found that a CIE chromaticity coordinate was (x, y)=(0.17, 0.31) and the light-emitting element of this embodiment exhibited blue light emission with favorable color purity.

### Embodiment 12

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using CuPc by an evaporation method. The first layer 303 was formed so as to be 20 nm thick. This first layer 303 serves as a hole injecting layer when a light-emitting element is operated.

Then, a second layer 304 was formed over the first layer 303 by using NPB by an evaporation method. The second layer 304 was formed so as to be 40 nm thick. This second layer 304 serves as a hole transporting layer when the light-emitting element is operated.

Next, a third layer 305 containing DPCzPA and PCABPA was formed over the second layer 304 by a co-evaporation method. The third layer 305 was formed so that its thickness was 40 nm and a mass ratio of DPCzPA to PCABPA was 1:0.04. Accordingly, PCABPA is in such a state that PCABPA is dispersed in a layer containing DPCzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

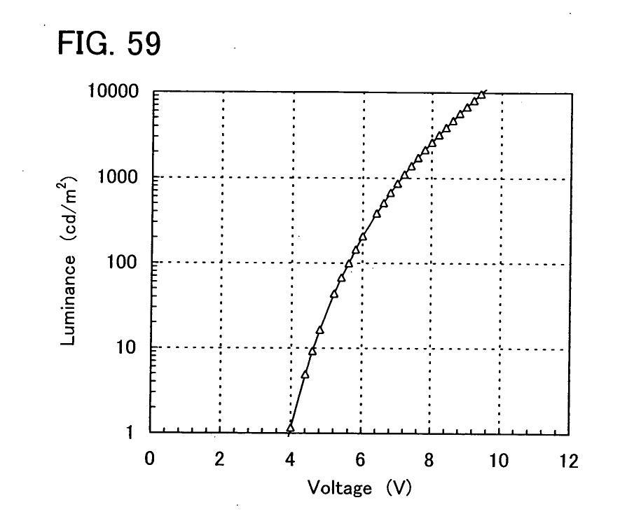

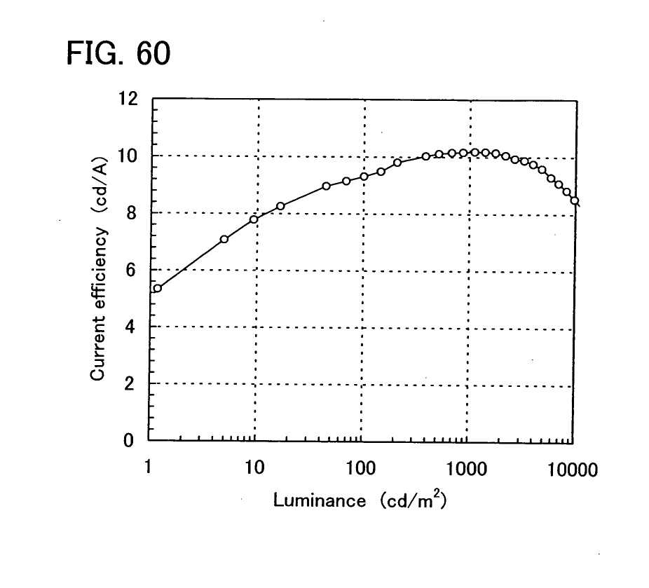

The measurement results are shown in FIGS. 59 and 60. FIG. 59 shows the measurement result of a voltage-luminance characteristic, and FIG. 60 shows the measurement result of a luminance-current efficiency characteristic. In FIG. 59, the horizontal axis indicates voltage (V) and the vertical axis indicates luminance (cd/m²). In FIG. 60, the horizontal axis indicates luminance (cd/m²) and the vertical axis indicates current efficiency (cd/A).

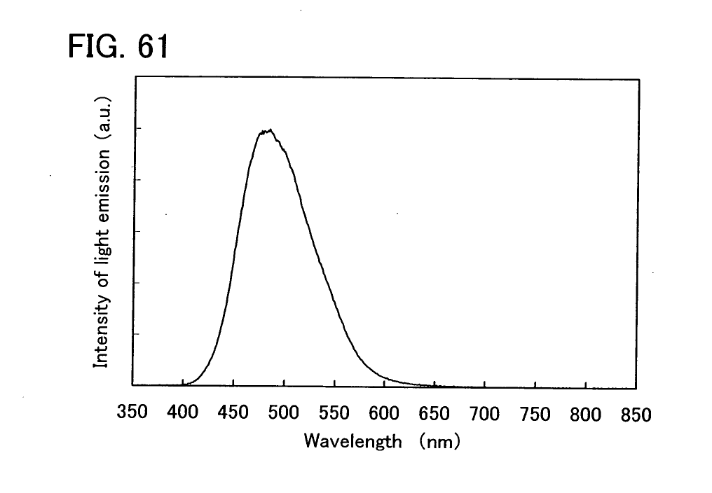

A light emission spectrum of the light-emitting element manufactured in this embodiment is shown in FIG. 61. In FIG. 61, the horizontal axis indicates a wavelength (nm) and the vertical axis indicates intensity (arbitrary unit). According to FIG. 61, it was found that the light-emitting element of this embodiment had a peak of the light emission spectrum at 485 nm and exhibited blue light emission. Furthermore, it was found that a CIE chromaticity coordinate was (x, y)=(0.17, 0.31) and the light-emitting element of this embodiment exhibited blue light emission with favorable color purity.

### Embodiment 13

In this embodiment, a manufacturing method of a light-emitting element using PCABPA synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using CuPc by an evaporation method. The first layer 303 was formed so as to be 20 nm thick. This first layer 303 serves as a hole injecting layer when a light-emitting element is operated.

Then, a second layer 304 was formed over the first layer 303 by using NPB by an evaporation method. The second layer 304 was formed so as to be 40 nm thick. This second layer 304 serves as a hole transporting layer when the light-emitting element is operated.

Next, a third layer 305 containing t-BuDNA and PCABPA was formed over the second layer 304 by a co-evaporation method. The third layer 305 was formed so that its thickness was 40 nm and a mass ratio of t-BuDNA to PCABPA was 1:0.04. Accordingly, PCABPA is in such a state that PCABPA is dispersed in a layer containing t-BuDNA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 10 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to aground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

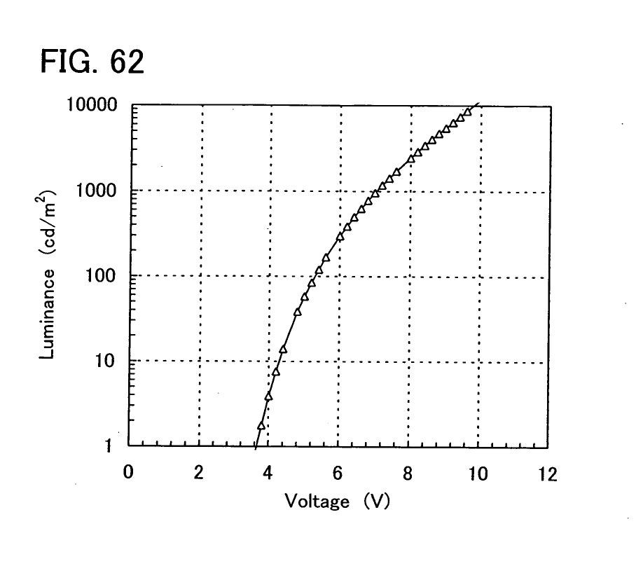

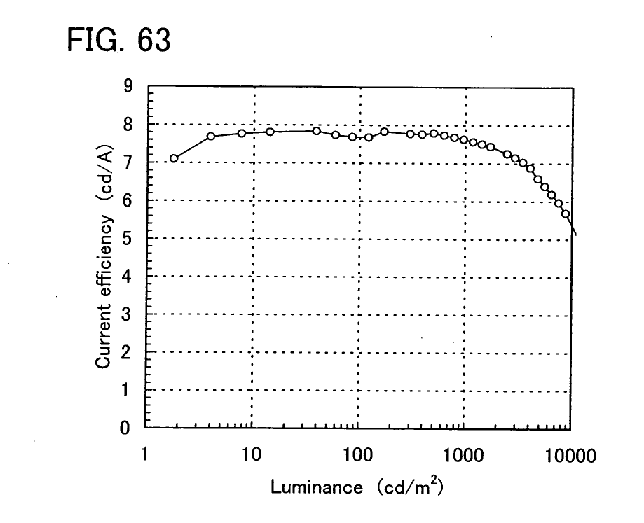

The measurement results are shown in FIGS. 62 and 63. FIG. 62 shows the measurement result of a voltage-luminance characteristic, and FIG. 63 shows the measurement result of a luminance-current efficiency characteristic. In FIG. 62, the horizontal axis indicates voltage (V) and the vertical axis indicates luminance (cd/m²). In FIG. 63, the horizontal axis indicates luminance (cd/m²) and the vertical axis indicates current efficiency (cd/A).

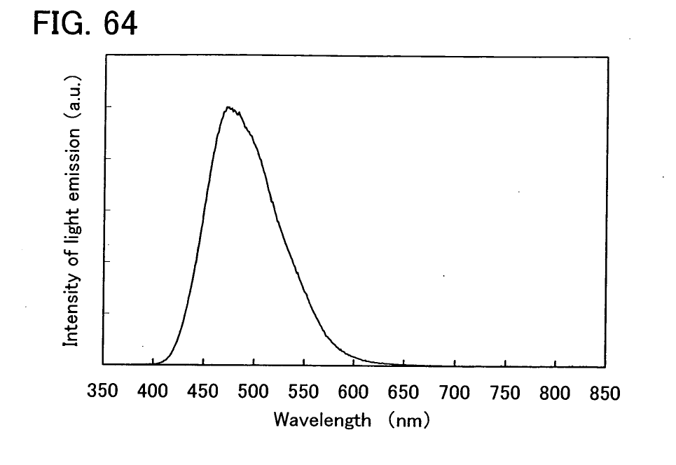

A light emission spectrum of the light-emitting element manufactured in this embodiment is shown in FIG. 64. In FIG. 64, the horizontal axis indicates a wavelength (nm) and the vertical axis indicates intensity (arbitrary unit). According to FIG. 64, it was found that the light-emitting element of this embodiment had a peak of the light emission spectrum at 476 nm and exhibited blue light emission. Furthermore, it was found that a CIE chromaticity coordinate was (x, y)=(0.16, 0.28) and the light-emitting element of this embodiment exhibited blue light emission with favorable color purity.

### Embodiment 14

In this embodiment, a manufacturing method of a light-emitting element using PCzPCA1 synthesized in Synthesis Example 3 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 containing NPB and molybdenum oxide was formed over the first electrode 302 by a co-evaporation method. The first layer 303 was formed so that its thickness was 40 nm and a mass ratio of NPB to molybdenum oxide was 4:1. Further, molybdenum oxide used as an evaporation material is molybdic anhydride. This first layer 303 serves as a hole generating layer when the light-emitting element is operated.

Then, a second layer 304 was formed over the first layer 303 by using PCzPCA1 which was synthesized in Synthesis Example 4 by an evaporation method. The second layer 304 was formed so as to be 20 nm thick. This second layer 304 serves as a hole transporting layer when the light-emitting element is operated.

Next, a third layer 304 was formed over the second layer 304 by using t-BuDNA by an evaporation method. The third layer 305 was formed so as to be 40 nm thick. This third layer 305 serves as a light-emitting layer when the light-emitting element is operated. In the light-emitting element of this embodiment, t-BuDNA serves as a ground substrate for forming the third layer 305 as well as a light-emitting substance.

Then, a fourth layer 306 was formed over the third layer 305 by using Alq₃ by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited t-BuDNA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

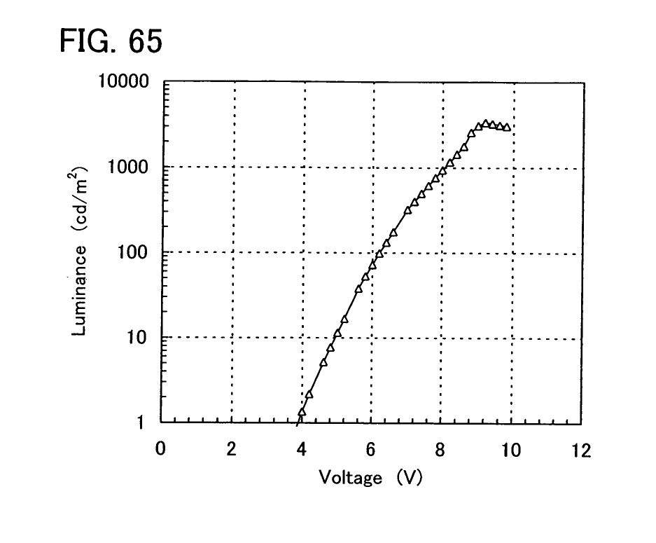

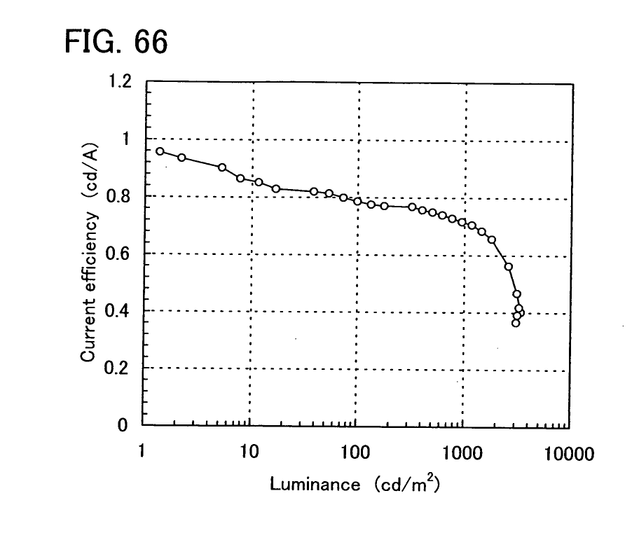

The measurement results are shown in FIGS. 65 and 66. FIG. 65 shows the measurement result of a voltage-luminance characteristic, and FIG. 66 shows the measurement result of a luminance-current efficiency characteristic. In FIG. 65, the horizontal axis indicates voltage (V) and the vertical axis indicates luminance (cd/m²). In FIG. 66, the horizontal axis indicates luminance (cd/m²) and the vertical axis indicates current efficiency (cd/A).

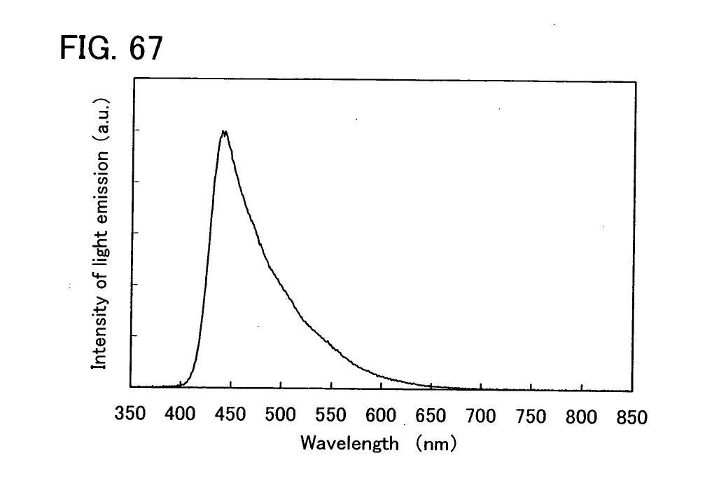

A light emission spectrum of the light-emitting element manufactured in this embodiment is shown in FIG. 67. In FIG. 67, the horizontal axis indicates a wavelength (nm) and the vertical axis indicates intensity (arbitrary unit). According to FIG. 67, it was found that the light-emitting element of this embodiment had a peak of the light emission spectrum at 443 nm and exhibited blue light emission. Furthermore, it was found that a CIE chromaticity coordinate was (x, y)=(0.17, 0.16) and the light-emitting element of this embodiment exhibited blue light emission with good color purity, which is derived from t-BuDNA.

### Embodiment 15

In this embodiment, a manufacturing method of a light-emitting element using PCzPCA1 synthesized in Synthesis Example 4 as a hole transporting material and PCABPA synthesized in Synthesis Example 3 as a light-emitting substance, and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 was formed over the first electrode 302 by using DNTPD by an evaporation method. The first layer 303 was formed so as to be 50 nm thick. This first layer 303 serves as a hole injecting layer when a light-emitting element is operated.

Then, a second layer 304 was formed over the first layer 303 by using PCzPCAl which was synthesized in Synthesis Example 4 by an evaporation method. The second layer 304 was formed so as to be 10 nm thick. This second layer 304 serves as a hole transporting layer when the light-emitting element is operated.

Next, a third layer 305 containing CzPA and PCABPA was formed over the second layer 304 by a co-evaporation method. The third layer 305 was formed so that its thickness was 40 nm and a mass ratio of CzPA to PCABPA was 1:0.03. Accordingly, PCABPA is in such a state that PCABPA is dispersed in a layer containing CzPA as its main component. This third layer 305 serves as a light-emitting layer when the light-emitting element is operated. Further, PCABPA serves as a light-emitting substance.

Then, a fourth layer 306 was formed over the third layer 305 by using Alq₃ by an evaporation method. The fourth layer 306 was formed so as to be 20 nm thick. This fourth layer 306 serves as an electron transporting layer when the light-emitting element is operated.

Subsequently, a fifth layer 307 was formed over the fourth layer 306 by using calcium fluoride by an evaporation method. The fifth layer 307 was formed so as to be 1 nm thick. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited PCABPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

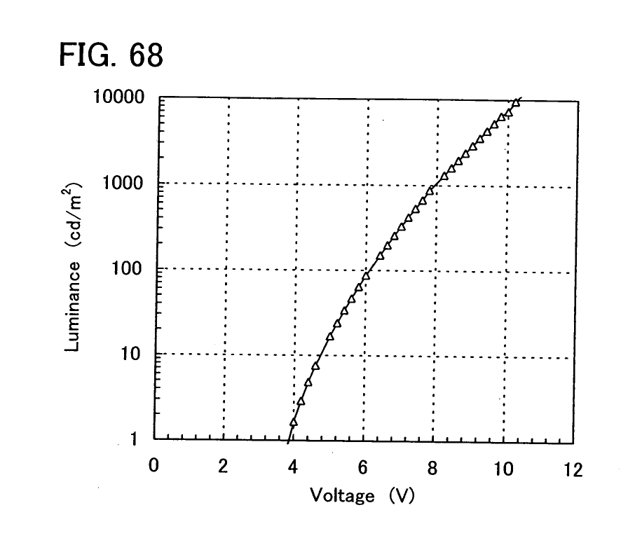

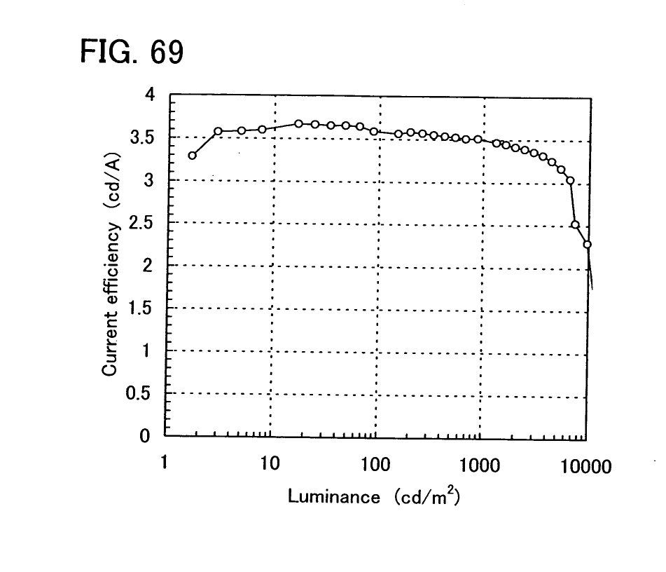

The measurement results are shown in FIGS. 68 and 69. FIG. 68 shows the measurement result of a voltage-luminance characteristic, and FIG. 69 shows the measurement result of a luminance-current efficiency characteristic. In FIG. 68, the horizontal axis indicates voltage (V) and the vertical axis indicates luminance (cd/m²). In FIG. 69, the horizontal axis indicates luminance (cd/m²) and the vertical axis indicates current efficiency (cd/A).

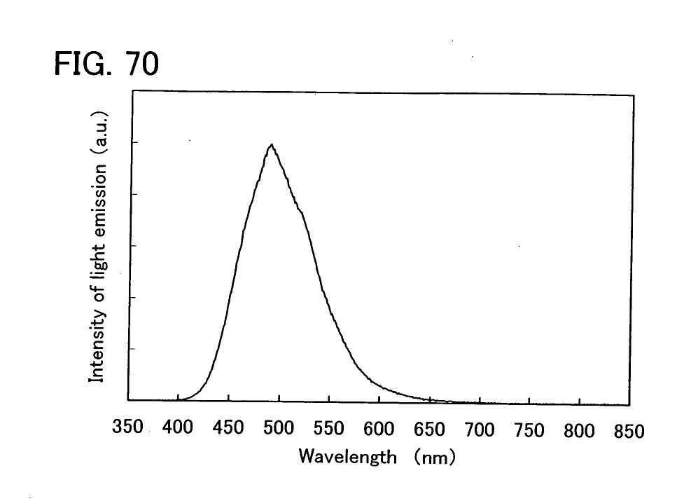

A light emission spectrum of the light-emitting element manufactured in this embodiment is shown in FIG. 70. In FIG. 70, the horizontal axis indicates wavelength (nm) and the vertical axis indicates intensity (arbitrary unit). According to FIG. 70, it was found that the light-emitting element of this embodiment had a peak of the light emission spectrum at 490 nm and exhibited blue light emission. Furthermore, it was found that a CIE chromaticity coordinate was (x, y)=(0.18, 0.36) and the light-emitting element of this embodiment exhibited blue light emission with favorable color purity, which is derived from PCABPA.

### Embodiment 16

A change in luminance with accumulation of light emission time and a change in operation voltage with accumulation of light emission time of each of the light emitting elements manufactured in Embodiments 6 and 7 were examined. A measurement was performed as follows. The manufactured light-emitting element was moved into a glove box under a nitrogen atmosphere to be sealed by using a sealing material in the same glove box. Then, current density required for light emission at luminance of 500 cd/m² in an initial state was measured first, and light continued to be emitted for certain amount of time by applying current of the current density required for light emission at luminance of 500 cd/m² in the initial state to plot light emission luminance and applied voltage by the elapsed time. It is to be noted that the current density required for light emission at luminance of 500 cd/m² was 5.85 mA/cm² as for the light-emitting element of Embodiment 6, and 5.5 mA/cm² as for the light-emitting element of Embodiment 7. Further, the measurement was performed in an atmosphere at a room temperature (approximately 25° C.).

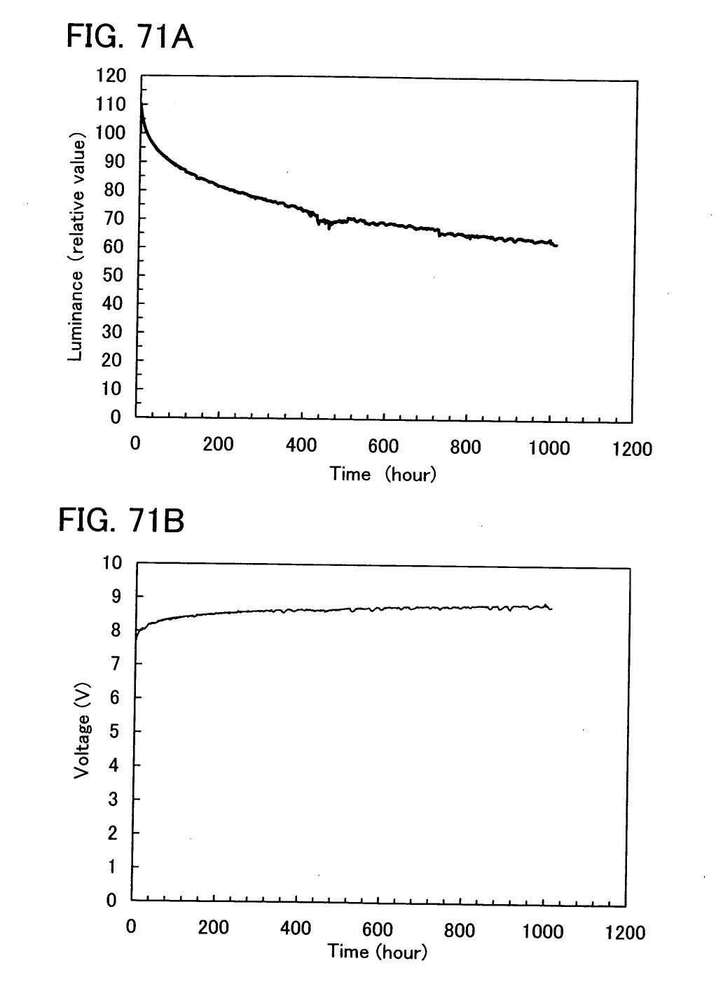

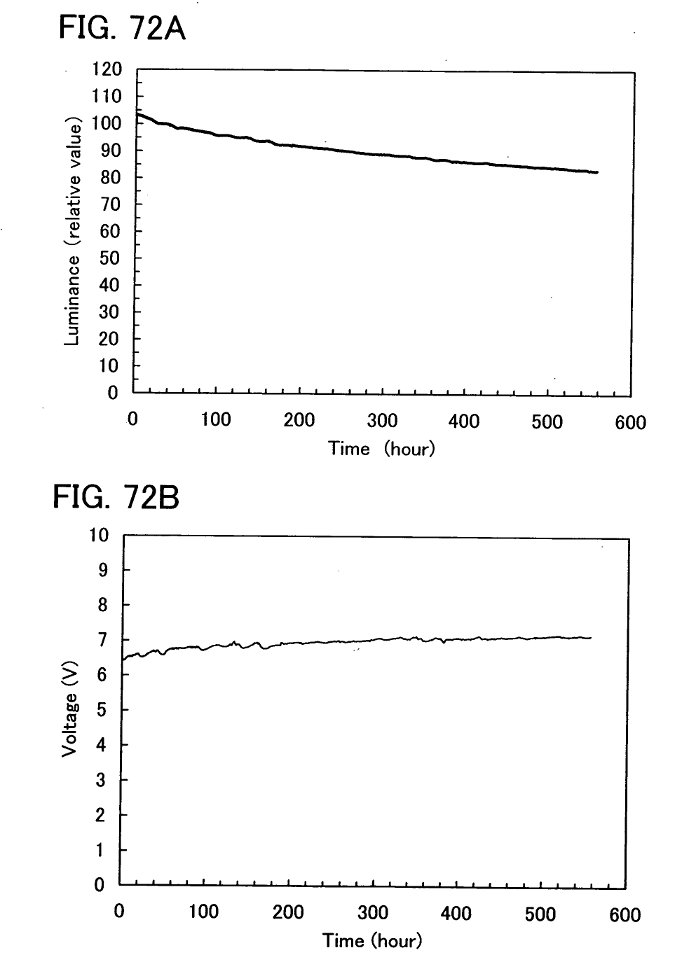

The measurement results of Embodiment 6 are shown in FIGS. 71A and 71B, and the measurement results of Embodiment 7 are shown in FIGS. 72A and 72B. Each of FIGS. 71A and 72A is a view showing a change in luminance with accumulation of the light emission time, and the horizontal axis indicates light emission time (hour) and the vertical axis indicates luminance (a relative value to initial luminance when the initial luminance is set to be 100). In addition, each of FIGS. 71B and 72B is a view showing a change in operation voltage with accumulation of the light emission time, and the horizontal axis indicates light emission time (hour) and the vertical axis indicates voltage (V) applied for applying current of the current density required for light emission at luminance of 500 cd/m² in an initial state.

According to FIGS. 71A and 72A, it is found that the both light-emitting elements of Embodiments 6 and 7 have little decrease in luminance with accumulation of the light emission time and that the light-emitting element of the present invention has favorable life duration. In addition, according to FIGS. 71B and 72B, it is found that the both light-emitting elements of Embodiments 6 and 7 have little increase in voltage with accumulation of the light emission time, that is, the light-emitting element of the present invention is a favorable element having little increase in resistance with accumulation of the light emission time.

### Embodiment 17

As one embodiment of the present invention, a synthesis method of N-[(4-biphenyl)carbazol-3-yl]-N-phenylamine (abbreviation: BCA) which is represented by a structural formula (40) and a synthesis method of 3-{N-[9-(4-biphenylyl)carbazol-3-yl]-N-phenylamino}-9-(4-biphenyl)carbazole (abbreviation: BCzBCA1) using BCA will be explained.

[Step 1]

First, a synthesis method of 9-(4-biphenylyl)carbazole will be explained. A synthesis scheme of 9-(4-biphenylyl)carbazole is shown in (m-1).

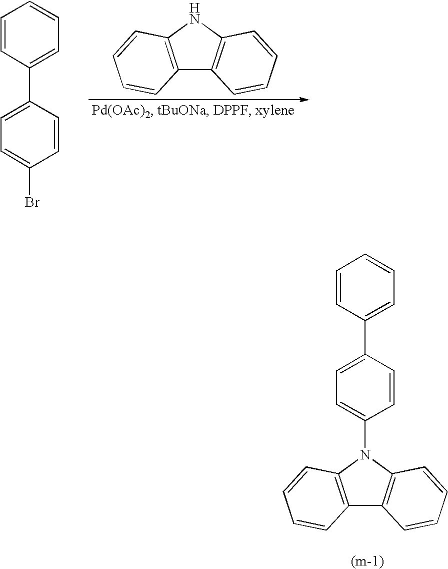

12 g (50 mmol) of 4-bromobiphenyl, 8.4 g (50 mmol) of carbazole, 230 mg (1 mmol) of palladium acetate (abbreviation: Pd(OAc)₂), 1.8 g (3.0 mmol) of 1,1-bis(diphenylphosphino)ferrocene (abbreviation: DPPF), and 13 g (180 mmol) of sodium-tert-butoxide (abbreviation: tert-BuONa) were put in a three-neck flask, and the atmosphere of the flask was substituted by nitrogen. Thereafter, 80 mL of dehydrated xylene was added thereto, and deaeration was performed. Under a nitrogen atmosphere, this was stirred while heating at 120° C. for 7.5 hours. After the termination of the reaction, about 600 mL of hot toluene was added to this suspension, and filtered twice through Florisil®, alumina, and Celite. The obtained filtrate was condensed, and hexane was added thereto to conduct recrystallization. This was filtered, and the residue was dried to obtain 14 g (yield: 87%) of 9-(4-biphenylyl)carbazole, which was cream-colored powder.

[Step 2]

Next, a synthesis method of 3-bromo-9-(4-biphenylyl)carbazole will be explained. A synthesis scheme of 3-bromo-9-(4-biphenylyl)carbazole is shown in (m-2).

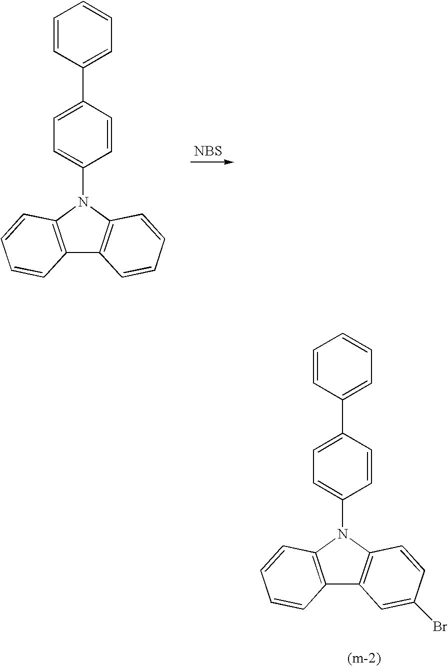

3.1 g (10 mmol) of 9-(4-biphenylyl)carbazole was dissolved in 100 mL of chloroform, and 1.8 g (10 mmol) of N-bromosuccinimide (abbreviation: NBS) was slowly added thereto. The mixture was stirred for about 24 hours, and then washed with water. Magnesium sulfate was added thereto to remove water and filtered to obtain filtrate. The filtrate was condensed, collected, and dried to obtain 3.7 g (yield: 95%) of 3-bromo-9-(4-biphenylyl)carbazole, which was beige powder.

[Step 3]

Next, a synthesis method of 3-iodo-9-(4-biphenylyl)carbazole will be explained. A synthesis scheme of 3-iodo-9-(4-biphenylyl)carbazole is shown in (m-3).

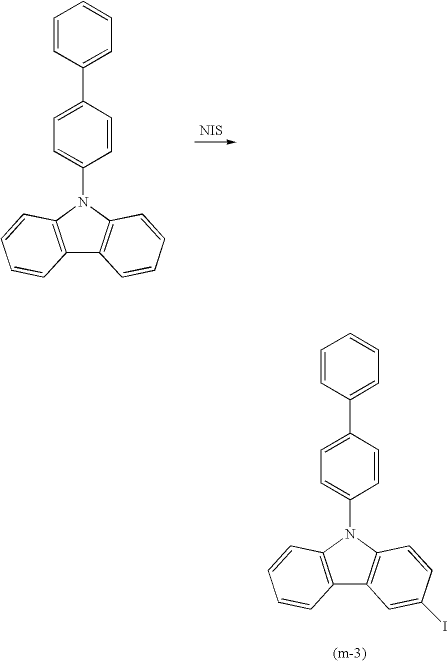

3.2 g (10 mmol) of 9-(4-biphenylyl)carbazole was dissolved in a mixed solution of 200 mL of glacial acetic acid, 200 mL of toluene, and 50 mL of ethyl acetate. 2.3 g (10 mmol) of N-iodosuccinimide (abbreviation: NIS) was slowly added thereto. The mixture was stirred for about 24 hours, and then washed with water, a sodium thiosulfate solution, and saturated saline. Magnesium sulfate was added thereto to remove water and filtered to obtain filtrate. The filtrate was condensed, and acetone and hexane were added thereto to conduct recrystallization with ultrasonic wave. This was filtered to obtain a residue. The residue was collected and dried to obtain 4.4 g (yield: 98%) of 3-iodo-9-(4-biphenylyl)carbazole, which was beige powder.

[Step 4]

A synthesis method of N-[(4-biphenylyl)carbazol-3-yl]-N-phenylamine (abbreviation: BCA) will be explained. A synthesis scheme of BCA is shown in (m-4).

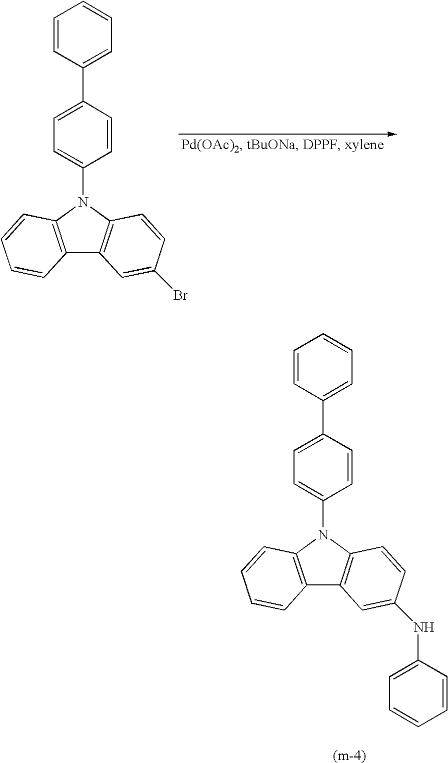

3.7 g (9.2 mmol) of 3-bromo-9-(4-biphenylyl)carbazole, 63 mg (0.3 mmol) of palladium acetate, 330 mg (0.6 mmol) of 1,1-bis(diphenylphosphino)ferrocene, and 1.5 g (15 mmol) of sodium-tert-butoxide were put in a three-neck flask and the atmosphere of the flask was substituted by nitrogen. Thereafter, 20 mL of dehydrated xylene was added, and deaeration was performed. Then, 9.3 g (10 mmol) of aniline was added thereto. This was stirred while heating at 130° C. under a nitrogen atmosphere for 4 hours. After the termination of the reaction, about 300 mL of hot toluene was added to this suspension, and the mixture was filtered through Florisil®, alumina, and Celite. The obtained filtrate was condensed and hexane was added thereto. Then, it was precipitated with ultrasonic wave. This was filtered, and the residue was dried to obtain 3.5 g (yield: 93%) of N-[(4-biphenylyl)carbazol-3-yl]-N-phenylamine (BCA), which was cream-colored powder.

[Step 5]

A synthesis method of 3-{N-[9-(4-biphenylyl)carbazol-3-yl]-N-phenylamino}-9-(4-biphenylyl)carbazole (abbreviation: BCzBCA1) will be explained. A synthesis scheme of BCzBCA1 is shown in (m-5).

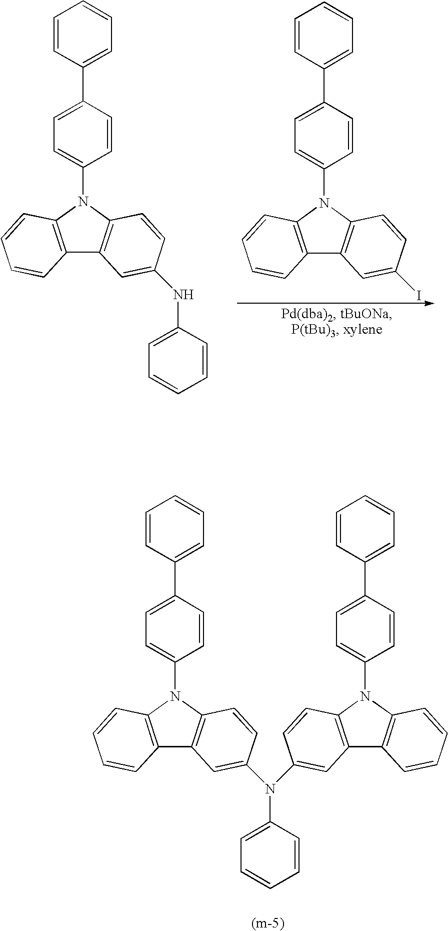

3.5 g (7.9 mmol) of 3-iodo-9-(4-biphenylyl)carbazole, 3.3 g (8.0 mmol) of N-[(4-biphenylyl)carbazol-3-yl]-N-phenylamine, 230 mg (0.4 mmol) of bis(dibenzylideneacetone)palladium(0) (abbreviation: Pd(dba)₂), and 1.2 g (12 mmol) of sodium-tert-butoxide (abbreviation: tert-BuONa) were put in a three-neck flask, and the atmosphere of the flask was substituted by nitrogen. Then, 30 mL of dehydrated xylene was added thereto, and deaeration was performed. 1.4 mL (1.2 mmol) of a hexane solution with 10 wt % of tri-tert-butylphosphine was added thereto. Under a nitrogen atmosphere, it was stirred while heating at 110° C. for 3 hours. After the termination of the reaction, about 500 mL of hot toluene was added to this suspension, and filtered through Florisil®, alumina, and Celite. The obtained filtrate was condensed and was obtained by silica gel column chromatography (toluene:hexane=1:1). This was condensed and hexane was added thereto. Then, it was precipitated with ultrasonic wave. 1.1 g (yield: 19%) of 3-{N-[9-(4-biphenylyl)carbazol-3-yl]-N-phenylamino}-9-(4-biphenylyl)carbazole (abbreviation.: BCzBCA1), which was cream-colored powder, was obtained

¹H-NMR data of this compound is shown below.

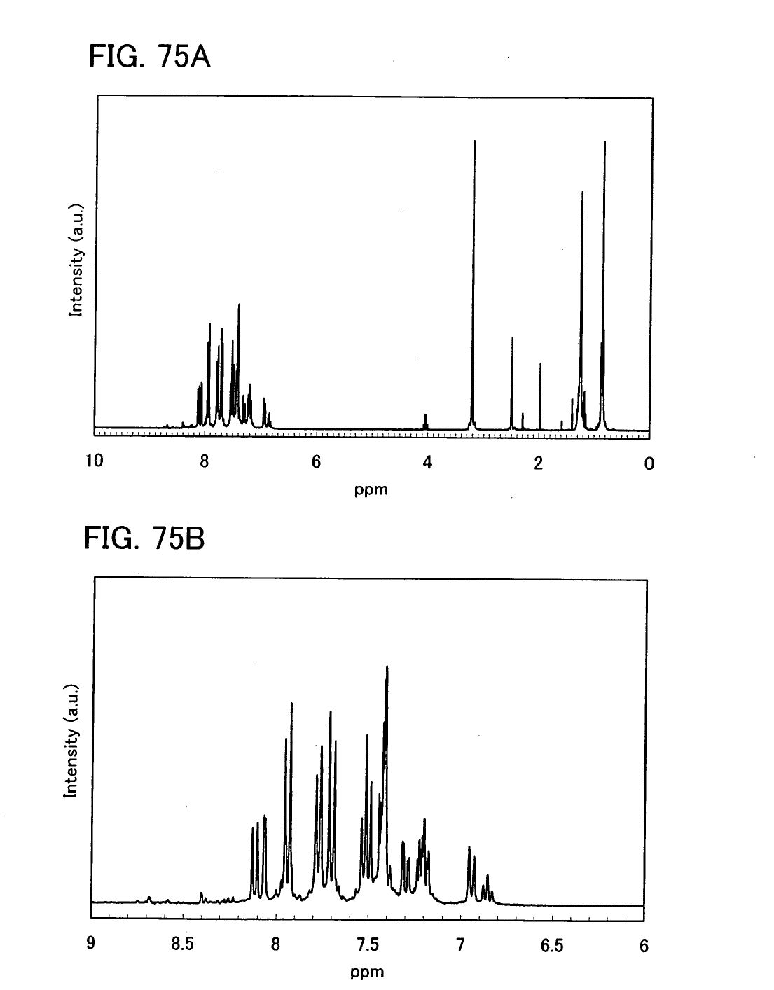

¹H-NMR (300 MHz, DMSO-d₆); δ=6.86 (t, J=7.2 Hz, 1 H), 6.94 (d, J=7.8 Hz, 2H), 7.18-7.24 (m, 4H), 7.30 (dd, J=8.9 Hz, 1.8, 2H), 7.41-7.54 (m, 12H), 7.70 (d, J=8.4 Hz, 4H), 7.77 (d, J=7.2 Hz, 4H), 7.94 (d, J=8.4 Hz, 4H), 8.06 (d, J=2.1 Hz, 2H), 8.12 (d, J=7.8 Hz, 2H) A ¹H-NMR chart is shown in FIG. 75A, and FIG. 75B is an enlarged chart of a portion of 6.0 to 9.0 ppm in FIG. 75A.

¹³C-NMR data is shown below.

¹³C-NMR (75.5 MHz, DMSO-d₆); δ=109.6, 110.7, 117.4, 119.4, 119.7, 119.8, 120.5, 120.5, 122.4, 123.7, 125.0, 126.2, 126.5, 126.8, 127.5, 128.1, 128.8, 136.0, 136.9, 139.1, 139.1, 140.6, 140.8, 149.3

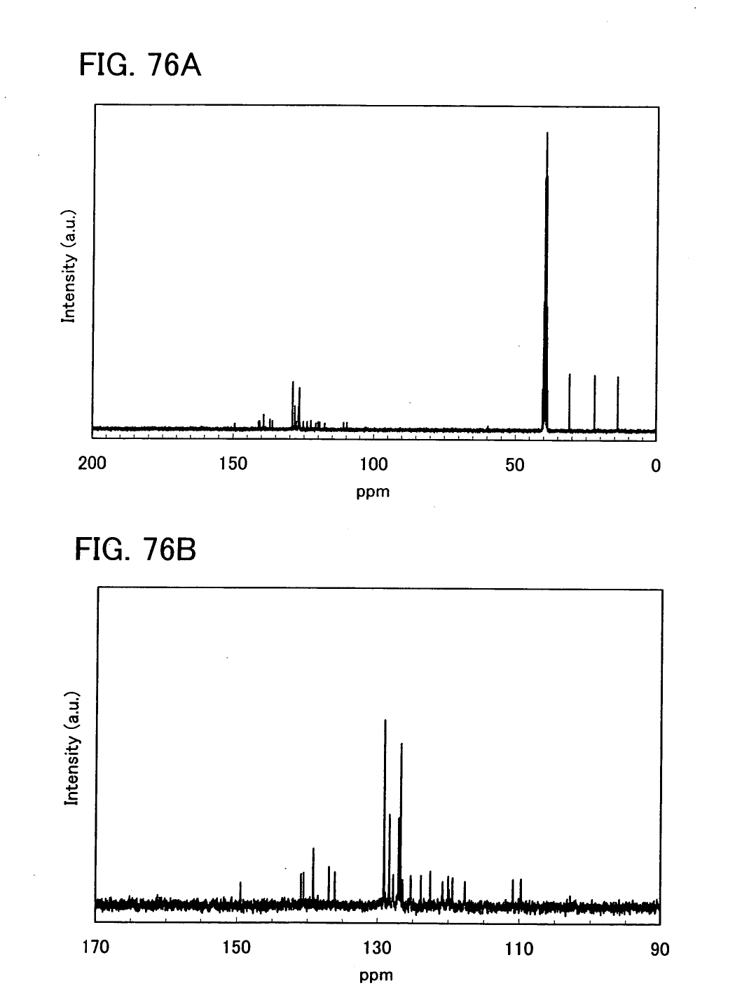

A ¹³C-NMR chart is shown in FIG. 76A, and FIG. 76B is an enlarged chart of a portion of 90 to 170 ppm in FIG. 76A.

A thermogravimetry-differential thermal analysis (TG-DTA) was performed on the obtained BCzBCA1. By using a thermo-gravimetric/differential thermal analyzer (TG/DTA 320, manufactured by Seiko Instruments Inc.), thermophysical properties were measured at a temperature rising rate of 10° C./min under a nitrogen atmosphere. As a result, from the relationship between the gravity and the temperature (thermogravimetry), the temperature T_{d}, at which the gravity becomes 95% or less with respect to the gravity at the start of the measurement, was 425° C. under normal pressure.

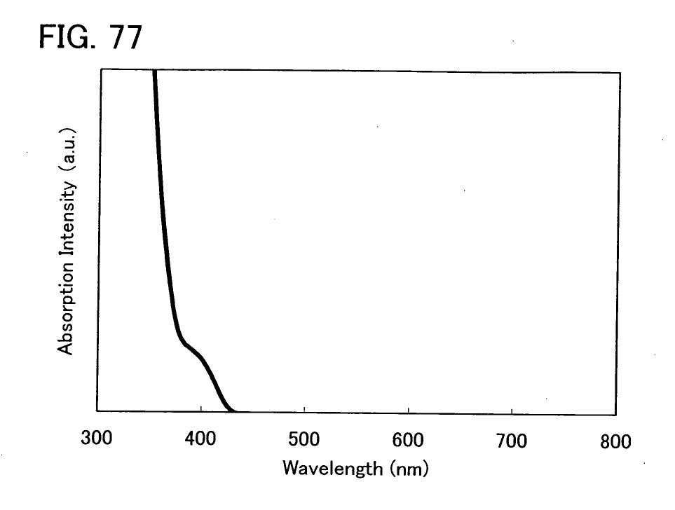

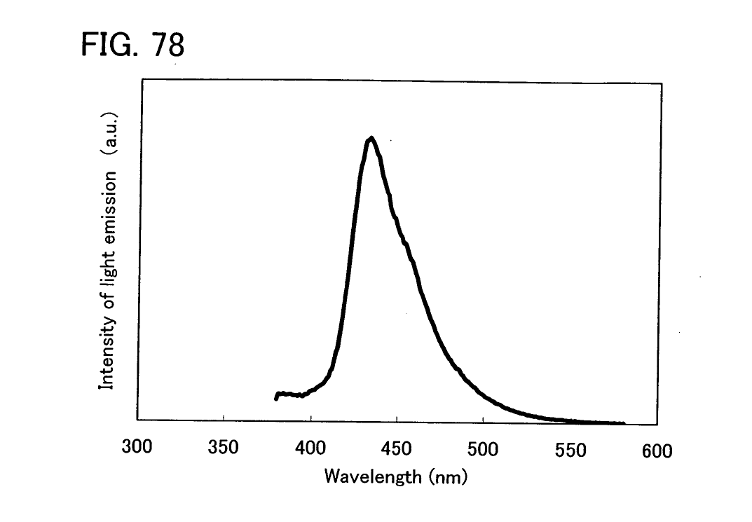

An absorption spectrum of a toluene solution of BCzBCA1 is shown in FIG. 77. An ultraviolet-visible spectrophotometer (V-550, manufactured by JASCO Corporation) was used for a measurement. The solution was put in a quartz cell to manufacture a sample, and the absorption spectrum, from which an absorption spectrum of quartz was subtracted, is shown in FIG. 77. In FIG. 77, the horizontal axis indicates a wavelength (nm) and the vertical axis indicates absorption intensity (arbitrary unit). The maximum absorption wavelength was 395 nm in the case of the toluene solution of BCzBCA1. An emission spectrum of the toluene solution of BCzBCA1 is shown in FIG. 78. In FIG. 78, the horizontal axis indicates a wavelength (nm) and the vertical axis indicates emission intensity (arbitrary unit). The maximum emission wavelength was 434 nm (an excited wavelength: 323 nm) in the case of the toluene solution of BCzBCA1.

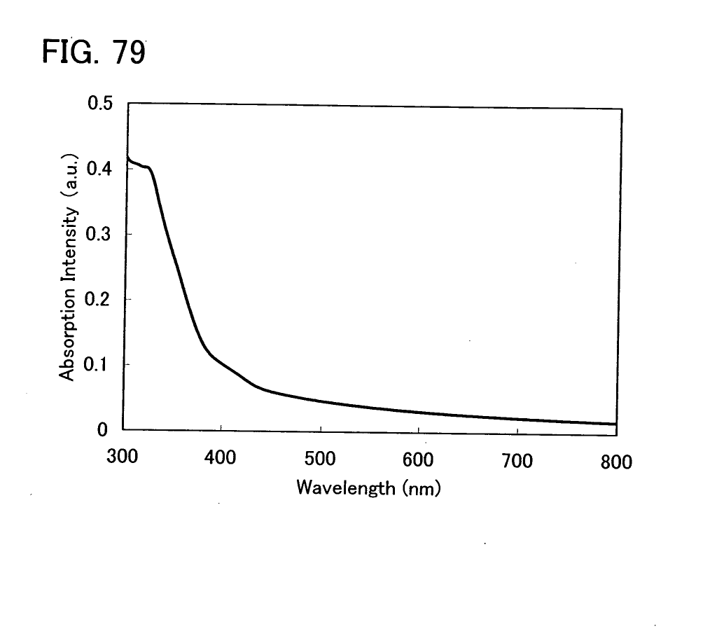

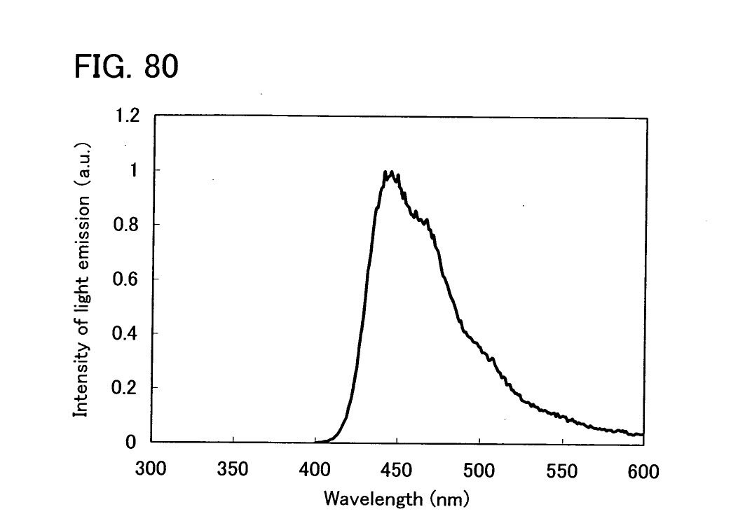

An absorption spectrum of a thin film of BCzBCA1 is shown in FIG. 79. An ultraviolet-visible spectrophotometer (V-550, manufactured by JASCO Corporation) was used for a measurement. The thin film was formed by being evaporated over a quartz substrate, and an absorption spectrum, from which an absorption spectrum of quartz was subtracted, is shown in FIG. 79. In FIG. 79, the horizontal axis indicates a wavelength (nm) and the vertical axis indicates absorption intensity (arbitrary unit). The maximum absorption a wavelength was 318 nm in the case of the thin film of BCzBCA1. An emission spectrum of the thin film of BCzBCA1 is shown in FIG. 80. In FIG. 80, the horizontal axis indicates a wavelength (nm) and the vertical axis indicates emission intensity (arbitrary unit). The maximum emission wavelength was 445 nm (an excited wavelength: 318 nm) in the case of the thin film of BCzBCA1.

A HOMO level and a LUMO level of BCzBCA1 in a state of a thin film were measured. A value of the HOMO level was obtained by converting a value of the ionization potential measured by using a photoelectron spectrometer (AC-2, manufactured by Riken Keiki Co., Ltd.) into a negative value. Also, a value of the LUMO level was obtained by adding an absorption edge of a thin film in FIG. 79 to the value of the HOMO level as an energy gap. As a result, the HOMO level and the LUMO level were −5.14 eV and −2.04 eV, respectively.

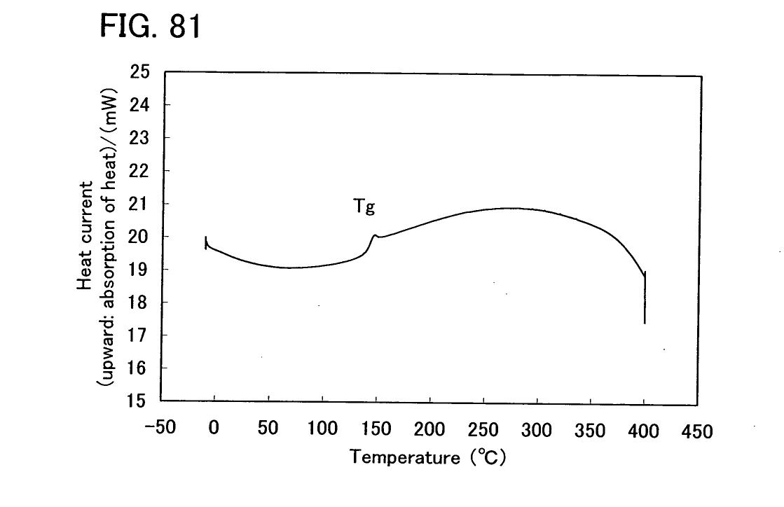

A glass transition temperature of the obtained compound BCzBCA1 was examined by using a differential scanning calorimetry (DSC) (Pyris 1 DSC, manufactured by Perkin Elmer Co., Ltd.). The measurement results by DSC are shown in FIG. 81. According to the measurement result, it was found that a glass transition point (Tg) of BCzBCA1 was 137° C. As described above, BCzBCA1 has the glass transition temperature as high as 137° C., and has favorable heat resistance. In addition, in FIG. 81, there is no peak showing crystallization of BCzBCA1, and thus it was found that BCzBCA1 is hard to be crystallized.

### Embodiment 18

In this embodiment, a manufacturing method of a light-emitting element using BCzBCA1 synthesized in Embodiment 17 as a hole transporting material, and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 containing NPB and molybdenum oxide was formed over the first electrode 302 by a co-evaporation method. The first layer 303 was formed so that its thickness was 50 nm and a mass ratio of NPB to molybdenum oxide was 4:1. Further, molybdenum oxide used as an evaporation material is molybdic anhydride. This first layer 303 serves as a hole generating layer when the light-emitting element is operated.

Then, a second layer 304 was formed over the first layer 303 by using BCzBCA1 which was synthesized in Embodiment 17 by an evaporation method. The second layer 304 was formed so as to be 10 nm thick. This second layer 304 serves as a hole transporting layer when the light-emitting element is operated.

Next, a third layer 305 containing Alq₃ and coumarin 6 was formed over the second layer 304 by a co-evaporation method. The third layer 305 was formed so that its thickness was 40 nm and a mass ratio of Alq₃ to coumarin 6 was 1:0.01. Accordingly, coumarin 6 is in a state that coumarin 6 is dispersed in a layer containing Alq₃ as its main component. This third layer 305 serves as a light-emitting layer when the light-emitting element is operated. Further, coumarin 6 serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 20 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited coumarin 6 returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

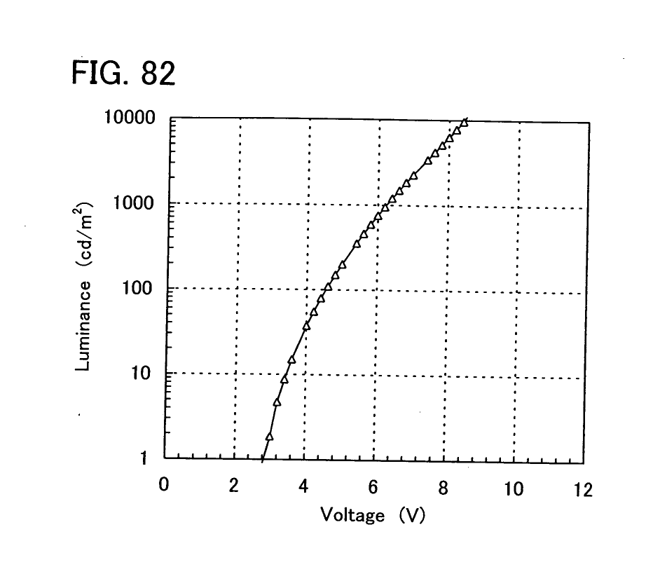

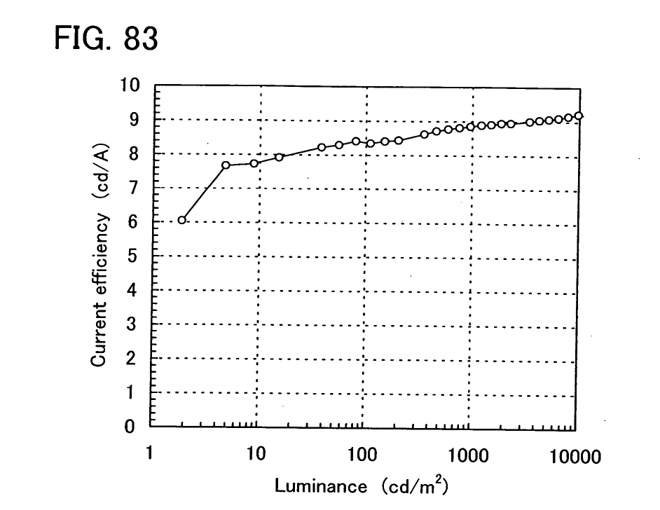

The measurement results are shown in FIGS. 82 and 83. FIG. 82 shows the measurement result of a voltage-luminance characteristic, and FIG. 83 shows the measurement result of a luminance-current efficiency characteristic. In FIG. 82, the horizontal axis indicates voltage (V) and the vertical axis indicates luminance (cd/m²). In FIG. 83, the horizontal axis indicates luminance (cd/m²) and the vertical axis indicates current efficiency (cd/A).

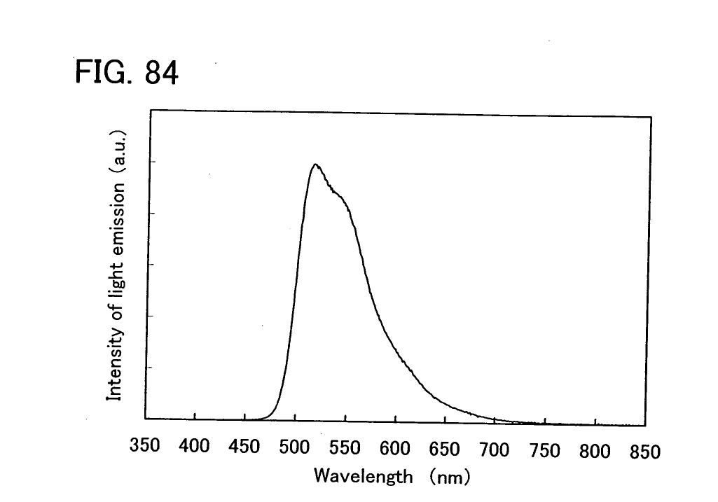

A light emission spectrum of the light-emitting element manufactured in this embodiment is shown in FIG. 84. In FIG. 84, the horizontal axis indicates a wavelength (nm) and the vertical axis indicates intensity (arbitrary unit). According to FIG. 84, it was found that the light-emitting element of this embodiment had a peak of the light emission spectrum at 516 nm and exhibited green light emission. Furthermore, it was found that a CIE chromaticity coordinate was (x, y)=(0.32, 0.62) and the light-emitting element of this embodiment exhibited green light emission with good color purity, which was derived from coumarin 6.

### Embodiment 19

In this embodiment, a manufacturing method of a light-emitting element using BCzBCA1 synthesized in Embodiment 17 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 containing BCzBCA1 which was synthesized in Embodiment 17 and molybdenum oxide was formed over the first electrode 302 by a co-evaporation method. The first layer 303 was formed so that its thickness was 50 nm and a mass ratio of BCzBCA1 to molybdenum oxide was 4:1. Further, molybdenum oxide used as an evaporation material is molybdic anhydride. This first layer 303 serves as a hole generating layer when the light-emitting element is operated.

Next, a second layer 304 was formed over the first layer 303 by using NPB by an evaporation method. The second layer 304 was formed so as to be 10 nm thick. This layer 304 serves as a hole transporting layer when the light-emitting element is operated.

Then, a third layer 305 containing CzPA and 9-(4-{N-[4-(9-carbazolyl)phenyl]-N-phenylamino}phenyl)-10-phenylanthracene (abbreviation: YGAPA) was formed over the second layer 304 by a co-evaporation method. The third layer 305 was formed so that its thickness was 30 nm and a mass ratio of CzPA to YGAPA was 1:0.04. Accordingly, YGAPA is in such a state that YGAPA is dispersed in a layer containing CzPA as its main component. This third layer 305 serves as a light-emitting layer when the light-emitting element is operated. Further, YGAPA serves as a light-emitting substance.

Then, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Next, a fifth layer 307 containing Alq₃ and Li was formed over the fourth layer 306 by a co-evaporation method. The fifth layer 307 was formed so that its thickness was 20 nm and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited YGAPA returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

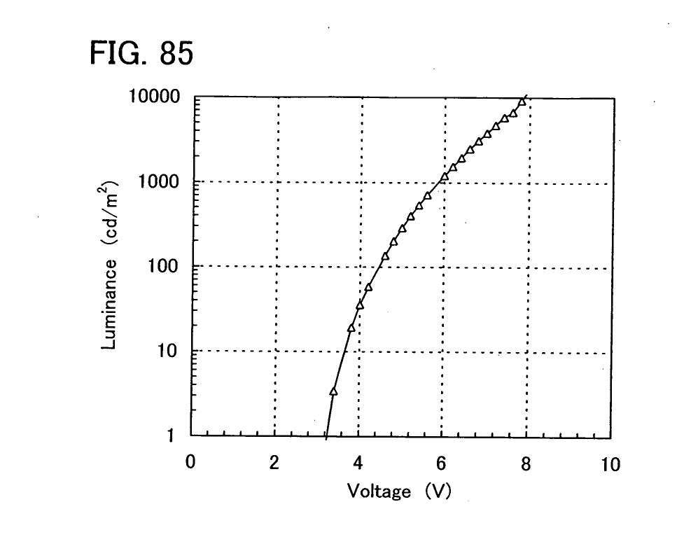

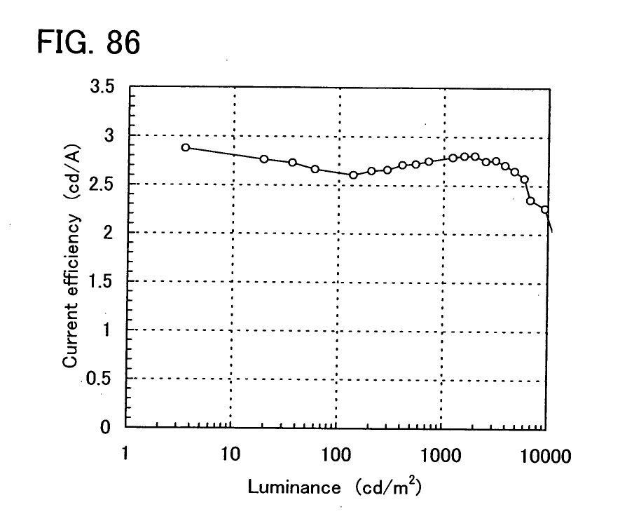

The measurement results are shown in FIGS. 85 and 86. FIG. 85 shows the measurement result of a voltage-luminance characteristic, and FIG. 86 shows the measurement result of a luminance-current efficiency characteristic. In FIG. 85, the horizontal axis indicates voltage (V) and the vertical axis indicates luminance (cd/m²). In FIG. 86, the horizontal axis indicates luminance (cd/m²) and the vertical axis indicates current efficiency (cd/A).

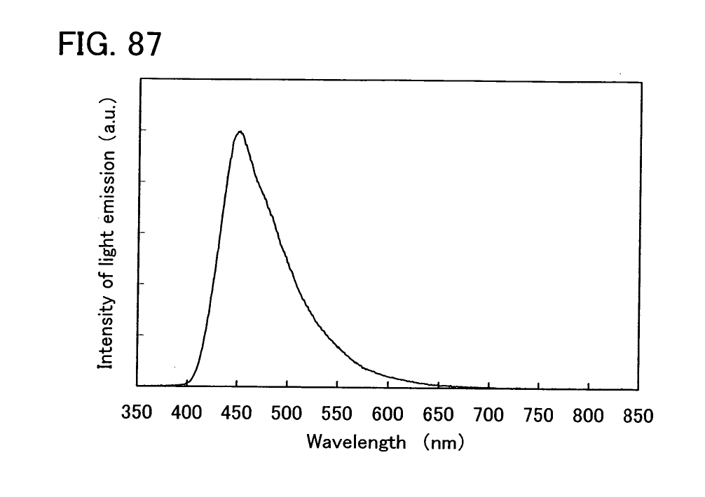

A light emission spectrum of the light-emitting element manufactured in this embodiment is shown in FIG. 87. In FIG. 87, the horizontal axis indicates a wavelength (nm) and the vertical axis indicates intensity (arbitrary unit). According to FIG. 87, it was found that the light-emitting element of this embodiment had a peak of the light emission spectrum at 451 nm and exhibited blue light emission. Furthermore, it was found that a CIE chromaticity coordinate was (x, y)=(0.16, 0.15) and the light-emitting element of this embodiment exhibited blue light emission with favorable color purity, which is derived from YGAPA.

### Embodiment 20

In this embodiment, a manufacturing method of a light-emitting element using BCzBCA1 synthesized in Embodiment 17 as a light-emitting substance and an operation characteristic thereof will be explained. It is to be noted that the light-emitting element of this embodiment is the same as the light-emitting element of Embodiment 4 in terms of having a structure in which five layers each including a different substance forming the layer and each having a different thickness are stacked between a first electrode and a second electrode; therefore, this embodiment will be explained with reference to FIG. 34 used for explaining Embodiment 4.

As shown in FIG. 34, indium tin oxide containing silicon oxide was deposited over a glass substrate 301 by a sputtering method to form a first electrode 302. The first electrode 302 was formed so as to be 110 nm thick. Further, the electrode was formed so as to be a square having a size of 2 mm×2 mm.

Next, the glass substrate 301 over which the first electrode 302 was formed was fixed to a holder provided inside a vacuum evaporation apparatus so that the surface over which the first electrode was formed faced downward.

Subsequently, the vacuum apparatus was evacuated to decrease pressure so as to be 10⁻⁴ Pa, and thereafter a first layer 303 containing BCzBCA1 which was synthesized in Embodiment 17 and molybdenum oxide was formed over the first electrode 302 by a co-evaporation method. The first layer 303 was formed so that its thickness was 50 nm and a mass ratio of BCzBCA1 to molybdenum oxide was 4:1. Further, molybdenum oxide used as an evaporation material is molybdic anhydride. This first layer 303 serves as a hole generating layer when the light-emitting element is operated.

Next, a second layer 304 was formed over the first layer 303 by using NPB by an evaporation method. The second layer 304 was formed so as to be 10 nm thick. This layer 304 serves as a hole transporting layer when the light-emitting element is operated.

Then, a third layer 305 containing Alq₃ and coumarin 6 was formed over the second layer 304 by a co-evaporation method. The third layer 305 was formed so that its thickness was 40 nm and a mass ratio of Alq₃ to coumarin 6 was 1 0.01. Accordingly, coumarin 6 is in such a state that coumarin 6 is dispersed in a layer containing Alq₃ as its main component. This third layer 305 serves as a light-emitting layer when the light-emitting element is operated. Further, coumarin 6 serves as a light-emitting substance.

Next, a fourth layer 306 was formed over the third layer 305 by using Alq₃ by an evaporation method. The fourth layer 306 was formed so as to be 10 nm thick. This fourth layer 306 serves as an electron transporting layer when the light-emitting element is operated.

Subsequently, a fifth layer 307 containing Alq₃ and Li was formed over the fourth layer 306 by an evaporation method. The fifth layer 307 was formed so that its thickness was 20 nm thick and a mass ratio of Alq₃ to Li was 1:0.01. This fifth layer 307 serves as an electron injecting layer when the light-emitting element is operated.

Next, a second electrode 308 was formed over the fifth layer 307 by using aluminum by an evaporation method. The second electrode 308 was formed so as to be 200 nm thick.

As for the light-emitting element manufactured as described above, current flows when voltage is applied so that potential of the first electrode 302 is higher than that of the second electrode 308, excitation energy is generated when electrons and holes are recombined in the third layer 305 serving as the light-emitting layer, and light is emitted when excited coumarin 6 returns to a ground state.

After the light-emitting element was sealed so as not to be exposed to the air under a nitrogen atmosphere in a glove box, an operation characteristic of the light-emitting element was measured. It is to be noted that the measurement was performed at a room temperature (an atmosphere in which 25° C. was kept).

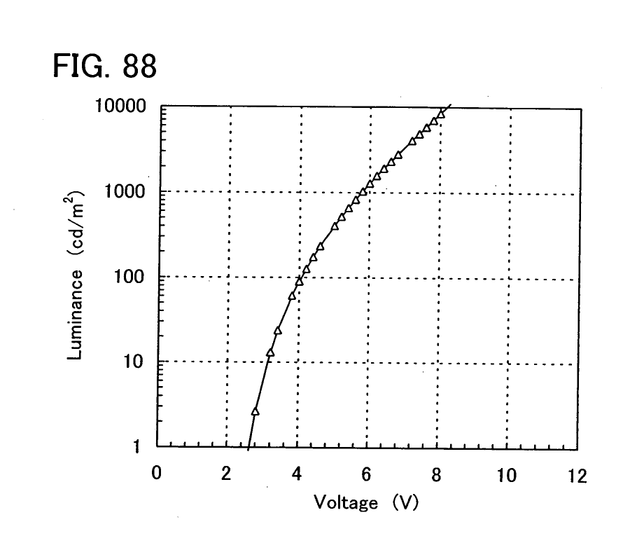

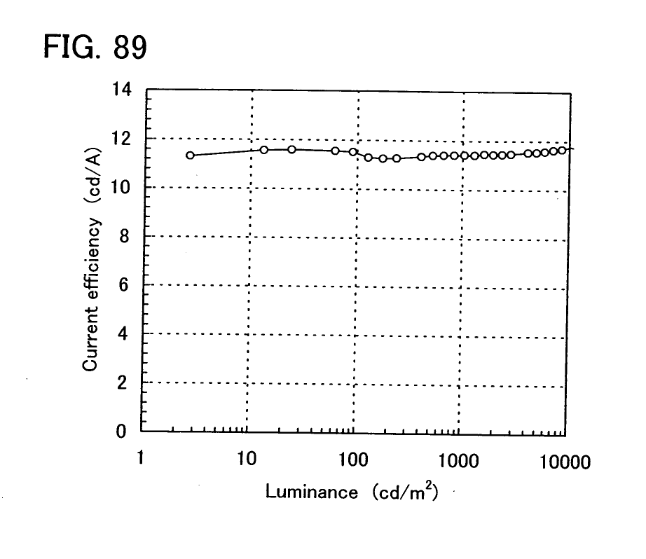

The measurement results are shown in FIGS. 88 and 89. FIG. 88 shows the measurement result of a voltage-luminance characteristic, and FIG. 89 shows the measurement result of a luminance-current efficiency characteristic. In FIG. 88, the horizontal axis indicates voltage (V) and the vertical axis indicates luminance (cd/m²). In FIG. 89, the horizontal axis indicates luminance (cd/m²) and the vertical axis indicates current efficiency (cd/A).

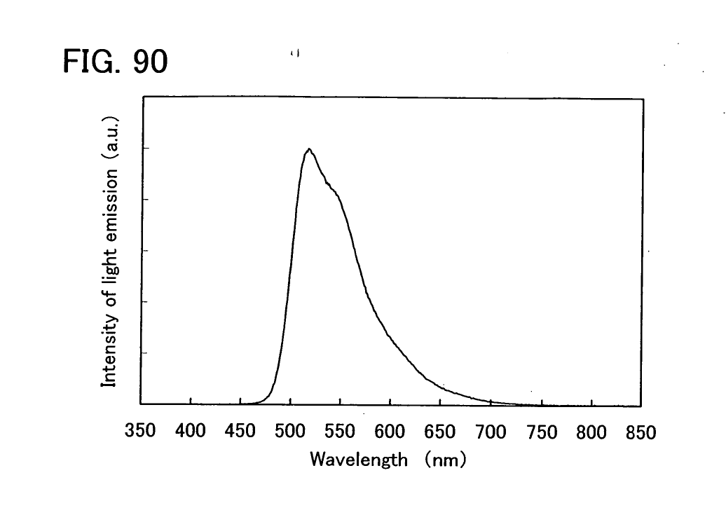

A light emission spectrum of the light-emitting element manufactured in this embodiment is shown in FIG. 90. In FIG. 90, the horizontal axis indicates a wavelength (nm) and the vertical axis indicates intensity (arbitrary unit). According to FIG. 90, it was found that the light-emitting element of this embodiment had a peak of the light emission spectrum at 516 nm and exhibited green light emission. Furthermore, it was found that a CIE chromaticity coordinate was (x, y)=(0.32, 0.62) and the light-emitting element of this embodiment exhibited green light emission with favorable color purity, which is derived from coumarin 6.

1. A carbazole derivative represented by a general formula (G-1): 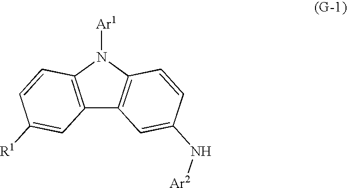
wherein each of Ar¹ and Ar² represents an aryl group having 1 to 12 carbon atoms, and
wherein R¹ represents any one of hydrogen, an alkyl group having 1 to 4 carbon atoms, and an aryl group having 6 to 12 carbon atoms.

2. A carbazole derivative represented by a general formula (G-2): 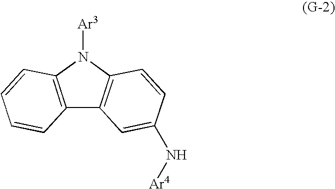
wherein each of Ar³ and Ar⁴ represents an aryl group having 1 to 12 carbon atoms.

3. A carbazole derivative represented by a structural formula:

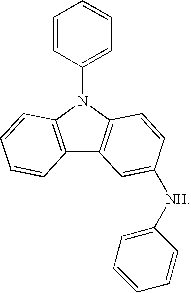

4. A carbazole derivative represented by a structural formula:

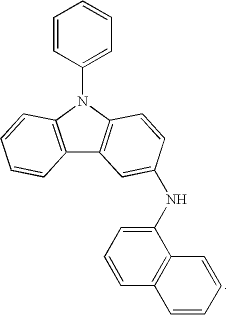

5. A light-emitting element material represented by a general formula (G-3): 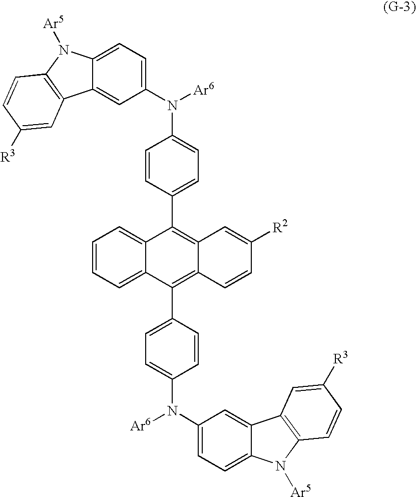
wherein each of Ar⁵ and Ar⁶ represents an aryl group having 1 to 12 carbon atoms,
wherein R represents any one of hydrogen, methyl, and tert-butyl, and
wherein R³ represents any one of hydrogen, an alkyl group having 1 to 4 carbon atoms, and an aryl group having 6 to 12 carbon atoms.

6. A light-emitting element material represented by a general formula (G-4): 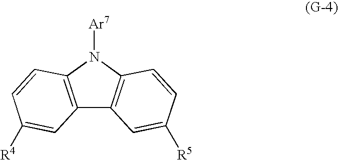
wherein Ar⁷ represents an aryl group having 1 to 12 carbon atoms,
wherein each of R⁴ and R⁵ represents hydrogen or a group represented by a general formula (G-5), and one of them is represented by the general formula (G-5): 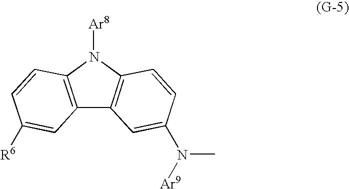
wherein each of Ar⁸ and Ar⁹ represents an aryl group having 1 to 12 carbon atoms, and
wherein R⁶ represents any one of hydrogen, an alkyl group having 1 to 4 carbon atoms, and an aryl group having 6 to 12 carbon atoms.

7. A light-emitting element comprising:
a light-emitting layer between electrodes,
wherein the light-emitting layer contains a light-emitting substance and a host,
wherein the light-emitting substance is represented by a general fromula (G-6): 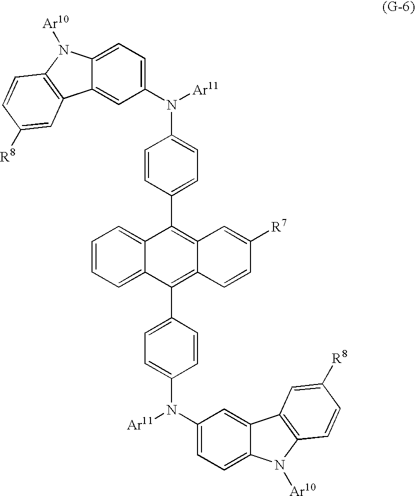
wherein the host has higher ionization potential than that of the light-emitting substance and a larger energy gap than that of the light-emitting substance,
wherein each of Ar¹⁰ and Ar¹¹ represents an aryl group having 1 to 12 carbon atoms,
wherein R⁷ represents any one of hydrogen, methyl, and tert-butyl, and
wherein R⁸ represents any one of hydrogen, an alkyl group having 1 to 4 carbon atoms, and an aryl group having 6 to 12 carbon atoms.

8. The light-emitting element according to claim 7, wherein the host has an electron transporting property which is higher than a hole transporting property.

9. A light-emitting element comprising:
a layer containing a carbazole derivative represented by a general formula (G-7): 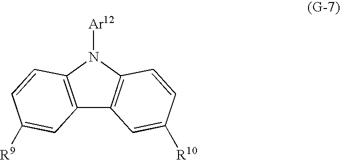
between a first electrode and a second electrode to be in contact with the first electrode,
wherein light is emitted when voltage is applied so that potential of the first electrode is higher than that of the second electrode,
wherein Ar¹² represents an aryl group having 1 to 12 carbon atoms
wherein each of R⁹ and R¹⁰ represents hydrogen or a group represented by a general formula (G-8), and one of them is represented by the general formula (G-8): 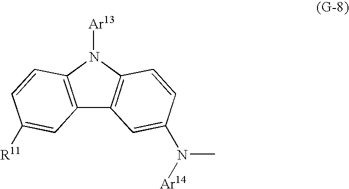
wherein each of Ar¹³ and Ar¹⁴ represents an aryl group having 1 to 12 carbon atoms, and
wherein R¹¹ represents any one of an alkyl group having 1 to 4 carbon atoms and an aryl group having 6 to 12 carbon atoms.

10. A light-emitting device comprising the light-emitting element according to claim 7.

11. A light-emitting device comprising the light-emitting element according to claim 9.

12. An electronic device comprising the light-emitting device according to claim 10 in a display protion or a lighting portion.

13. An electronic device comprising the light-emitting device according to claim 11 in a display protion or a lighting portion.
